(19) United States
(12) Patent Application Publication
(10) Pub. No.: US 2026/0240837 A1
(43) Pub. Date: Aug. 20, 2026

(54) Compositions Reducing the Expression or Activity of C1 GALT1 for Treating Ewing Sarcoma

(71) Applicant: University of Massachusetts Medical School, Worcester, MA (US)

(72) Inventors: Shahid Khursheed Banday, Worcester, MA (US); Michael R. Green, Boylston, MA (US)

(21) Appl. No.: 19/489,554

(22) PCT Filed: May 31, 2024
(86) PCT No.: PCT/US2024/032149

§ 371 Date: Dec. 2, 2025

Related U.S. Application Data
(60) Provisional application No. 63/506,048, filed on Jun. 2, 2023.

(51) Int. Cl. A61K 31/496; A61P 35/00

(52) U.S. Cl. CPC A61K 31/496; A61P 35/00

(57) ABSTRACT

The disclosure provides, in various embodiments, methods of treating Ewing sarcoma in a subject. The disclosure also provides, in various embodiments, methods of killing and/or reducing proliferation of an Ewing sarcoma tumor cell. The disclosure also provides, in various embodiments, methods of reducing expression of EWSR1::FLI1, reducing hedgehog pathway activity and/or reducing Smoothened (SMO) activity.

### RELATED APPLICATION

This application claims the benefit of U.S. Provisional Application No. 63/506,048, filed on Jun. 2, 2023. The entire teachings of the above application are incorporated herein by reference.

### INCORPORATION BY REFERENCE OF MATERIAL IN XML

This application incorporates by reference the Sequence Listing contained in the following extensible Markup Language (XML) file being submitted concurrently herewith: File name: 5439.1033001 Sequence Listing.xml; created May 31, 2024, 79.750 Bytes in size.

### BACKGROUND

Ewing sarcoma (ES) is an aggressive bone and soft-tissue cancer that occurs predominantly in children and young adults (Riggi et al., 2021). Current treatment of ES involves an intensive chemotherapy regimen followed by local tumor reduction through radiotherapy and/or surgery. Although outcomes have improved over the last several decades for ES patients with localized disease, little progress has been made in the treatment of patients with metastatic or recurrent disease, for which 5-year survival rates remain at a dismal 20-30% (Casey et al., 2019; Hesla et al., 2021). Notably, there are no approved targeted therapies for ES.

### SUMMARY

There is a critical need to develop new methods for treating Ewing sarcoma (ES). The disclosure provides such methods.

Provided herein, among other things, are methods of treating Ewing sarcoma in a subject, the methods comprising administering to the subject an effective amount of an agent or a pharmaceutically acceptable salt thereof that reduces expression of core 1 synthase, glycoprotein-N-acetylgalactosamine 3-beta-galactosyltransferase 1 (C1GALT1), reduces activity of C1GALT1, or reduces both expression of C1GALT1 and activity of C1GALT1, thereby treating Ewing sarcoma in the subject.

In some embodiments, an agent or a pharmaceutically acceptable salt thereof reduces expression of C1GALT1. In some embodiments, an agent or a pharmaceutically acceptable salt thereof reduces activity of C1GALT1.

In some embodiments, an agent or a pharmaceutically acceptable salt thereof:
a) reduces expression of Smoothened (SMO);
b) reduces O—N-acetylgalactosamine-glycosylation (O-GalNAc-glycosylation) of SMO;
c) reduces activity of SMO;
d) reduces stability of SMO;
e) increases ubiquitination of SMO;
f) reduces ligand-independent hedgehog signaling;
g) reduces expression of GLI family zinc finger 1 (GLI1);
h) reduces expression of GLI family zinc finger 2 (GLI2);
i) reduces binding of GLI1 to EWS RNA binding protein 1 (EWSR1);
j) reduces binding of GLI2 to EWSR1;
k) reduces binding of GLI1 to EWSR1 and Fli-1 proto-oncogene, ETS transcription factor (FLI1) fusion gene (EWSR1::FLI1);
1) reduces binding of GLI2 to EWSR1::FLI1;
m) reduces expression of EWSR1::FLI1;
n) reduces expression of an EWSR1-FLI1 fusion protein (EWSR1::FLI1) target gene;
o) reduces binding of GLI1 to EWSR1 and ETS transcription factor ERG (ERG) fusion gene (EWSR1::ERG);
p) reduces binding of GLI2 to EWSR1::ERG;
q) reduces expression of EWSR1::ERG;
r) reduces expression of an EWSR1-ERG fusion protein (EWSR1::ERG) target gene;
or any combination of the foregoing.

In some embodiments, an agent or a pharmaceutically acceptable salt thereof reduces expression of EWSR1::FLI1, EWSR1::ERG, or both, and:
a) reduces expression of H3 clustered histone 10 (H3C10), zinc finger and BTB domain containing 21 (ZBTB21), histone cell cycle regulator (HIRA), activating transcription factor 6 beta (ATF6B), linker for activation of T cells (LAT), dedicator of cytokinesis 11 (DOCK11), fucosyltransferase 3 (FUT3), 3-hydroxy-3-methylglutaryl-CoA lyase (HMGCL), C1GALT1, C1GALT1-specific chaperone 1 (C1GALT1C1), SMO, GLI1, GLI2 or any combination of the foregoing;
b) reduces activity of H3C10, ZBTB21, HIRA, ATF6B, LAT, DOCK11, FUT3, HMGCL, C1GALT1, C1GALT1C1, SMO, GLI1, GLI2 or any combination of the foregoing;
or both a) and b).

In some embodiments, an EWSR1::FLI1 target gene comprises G protein-coupled receptor kinase 5 (GRK5), nuclear receptor subfamily 0 group b member 1 (NR0B1), vaccinia-related kinase 1 (VRK1), cyclin-dependent kinase inhibitor 1A (CDKN1A), fos-related antigen 2 (FOSL2), forkhead box protein 01 (FOXO1), or any combination of the foregoing.

In some embodiments, an agent comprises itraconazole or an analog thereof.

In some embodiments, an agent or a pharmaceutically acceptable salt thereof is administered to a subject at about 0.0001 to about 100 mg per kilogram of body weight per day.

In some embodiments, an agent or a pharmaceutically acceptable salt thereof is administered to a subject:
a) at about 100 to about 1,200 mg per day;
b) at about 100 mg, about 200 mg, about 300 mg, about 400 mg, about 500 mg, or about 600 mg, once daily; or
c) at about 100 mg, about 200 mg, about 300 mg, about 400 mg, about 500 mg, or about 600 mg, twice daily.

In some embodiments, an agent or a pharmaceutically acceptable salt thereof is administered to a subject at about 10 mg/mL.

In some embodiments, an agent or a pharmaceutically acceptable salt thereof is administered to a subject:
a) for at least 1, 2, 3, 4, 5, 6, 7, 8, 9, 10, 11 or 12 weeks;
b) for about 1 day to about 12 weeks;
or both a) and b).

In some embodiments, a method further comprises administering to a subject a second agent, a chemotherapy, a radiation therapy, or any combination of the foregoing.

In some embodiments, an amount of an agent or a pharmaceutically acceptable salt thereof administered to a subject is sufficient to reduce tumor size, improve radiological response, prolong event-free survival, prolong disease-free survival, prolong metastasis-free survival, prolong overall survival, or any combination of the foregoing.

Also provided herein, among other things, are methods of treating Ewing sarcoma in a subject, comprising administering to the subject an effective amount of itraconazole, an analog thereof, or a pharmaceutically acceptable salt thereof.

In some embodiments, a subject is a mammal, optionally wherein a mammal is a human.

In some embodiments, a subject is newly diagnosed with metastatic Ewing sarcoma, non-metastatic Ewing sarcoma, or localized Ewing sarcoma.

In some embodiments, a subject has or is susceptible to metastatic Ewing sarcoma, relapsed or recurrent Ewing sarcoma, non-metastatic Ewing sarcoma, or localized Ewing sarcoma.

Also provided herein, among other things, are methods of killing and/or reducing proliferation of an Ewing sarcoma tumor cell, comprising contacting the cell with an agent that reduces expression of C1GALT1, reduces activity of C1GALT1, or reduces both expression of C1GALT1 and activity of C1GALT1.

In some embodiments, an agent comprises itraconazole or an analog thereof.

In some embodiments, a cell is a relapsed or recurrent Ewing sarcoma cell, a metastatic Ewing sarcoma cell, a non-metastatic Ewing sarcoma cell, a localized Ewing sarcoma cell, an Ewing sarcoma cell from a subject, an Ewing sarcoma cell from a cell line, or a combination of the foregoing.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-1F show construction and validation of a reporter cell line: A673/EWSR1::FLI1^{tdTomato}/Cas9/EGFP. Where applicable, the error bars indicate standard error of the mean (SEM); the P values were calculated using one-way ANOVA followed by Dunnett's multiple comparisons test; and **P<0.01.

FIG. 1A is a schematic illustration of the CRISPR/Cas9-mediated homology-directed repair strategy to insert the tdTomato reporter at the 3′ end of the endogenous fusion gene EWSR1::FLI1. UTR, untranslated region; LHR, left homology region; RHR, right homology region; 2A, 2A self-cleaving peptide; Neo, neomycin resistance cassette.

FIG. 1B shows a schematic illustration of the reverse transcription polymerase chain reaction (RT-PCR) strategy to confirm insertion of tdTomato at the 3′ end of FLI1 (top panel, arrows indicate the location of the primer pairs; and an RT-PCR analysis detecting the FLI1-tdTomato fusion in the A673/EWSR1::FLI1^{tdTomato} pooled population (Pooled popn) and three single A673/EWSR1::FLI1^{tdTomato} knock-in (KI) clones (KI clones 2-4) (bottom panel). Parental A673 cells were used as a negative control. KI clone 2 was selected for the reporter cell line. PCR product size is indicated in base pairs (bp).

FIG. 1C is a flow cytometry analysis showing the percentage of tdTomato-positive cells in the A673/EWSR1::FLI1^{tdTomato} clone 2 population. Parental A673 cells were used as a negative control. The results show that the vast majority of the A673/EWSR1::FLI1^{tdTomato} clone 2 population was tdTomato-positive.

FIG. 1D is a flow cytometry analysis of tdTomato in A673/EWSR1::FLI1^{tdTomato} cells expressing a non-silencing (NS), EWSR1, or FLI1 short hairpin RNA (shRNA). Left, representative histogram. Right, mean fluorescence intensity (MFI), normalized to the MFI observed in cells expressing the control NS shRNA. The results demonstrate that knockdown of EWSR1 or FLI1 (encoding the N- or C-terminal portion of the EWSR1::FLI1 fusion gene, respectively) substantially reduced expression of the EWSR1::FLI1^{tdTomato} reporter.

FIG. 1E is a flow cytometry analysis of a Cas9 activity assay. Parental A673 cells or the A673/EWSR1::FLI1^{tdTomato} clone 2 population stably expressing Cas9 were transduced with a reporter plasmid carrying a puromycin resistance marker, an EGFP reporter gene, and a single guide RNA (sgRNA) sequence specifically targeting the EGFP gene. In the absence of Cas9 (parental A673 cells), the cells both expressed EGFP (GFP) and were resistant to puromycin. In the presence of Cas9 (A673/Cas9/EWSR1::FLI1^{tdTomato} cells), the cells were puromycin resistant but did not express EGFP.

FIG. 1F is an immunoblot confirming Cas9 expression in the reporter cell line (A673/EWSR1::FLI1^{tdTomato}/Cas9/EGFP). Parental A673 cells were used as a negative control. β-actin (ACTB) was used as a loading control. Molecular weights are indicated in kDa.

FIGS. 2A-2D show a genome-scale CRISPR/Cas9 screen that identified factors that promote EWSR1::FLI1 expression. Where applicable: the error bars indicate SEM; the P values were calculated using an unpaired, two-tailed t-test; *P<0.05; **P<0.01; ***P<0.001.

FIG. 2A is a schematic illustration of the genome-scale CRISPR/Cas9-based screening strategy.

FIG. 2B is an immunoblot showing EWSR1::FLI1 protein levels in A673 cells expressing two independent shRNAs targeting each of the nine validated candidates, or, as controls, an NS or EWSR1 shRNA. EWSR1::FLI1 levels were monitoring using an anti-FLI1 antibody. ACTB was used as a loading control. Molecular weights are indicated in kDa.

FIG. 2C is a quantitative RT-PCR (qRT-PCR) analysis monitoring knockdown efficiency of the shRNAs targeting each of the nine candidates. The results were normalized to that obtained with a NS shRNA, which was set to 1 (not shown).

FIG. 2D is a qRT-PCR analysis monitoring EWSR1::FLI1 expression following knockdown of each of the nine candidates. EWSR1::FLI1 expression was detected using a primer pair spanning the fusion junction and is shown relative to that obtained with an NS control shRNA, which was set to 1.

FIGS. 3A-3C show that core 1 synthase, glycoprotein-N-acetylgalactosamine 3-beta-galactosyltransferase 1 (C1GALT1) is highly expressed in ES and is associated with decreased overall survival.

FIG. 3A is an immunohistochemistry analysis showing C1GALT1 and hemotoxylin and eosin (H&E) staining in two ES patient tumor samples, as well as normal bone trabecula and cartilage. Scale bars correspond to 50 μm.

FIG. 3B is a Kaplan-Meier analysis showing reduced overall survival probability in ES patients with high C1GALT1 expression compared to those with low C1GALT1 expression, derived from datasets GSE63157 (left) and GSE17679 (right).

FIG. 3C is an immunoblot showing C1GALT1 protein levels in normal human lung IMR90 fibroblasts, human osteosarcoma cell lines (Saos-2, MG-63, U-2 OS), and human ES cell lines (A673, SK-N-MC, TC-71, TC-106, TC-32). GAPDH was used as a loading control. Molecular weights are indicated in kDa.

FIGS. 4A-4F show that biological inhibition of C1GALT1 reduces EWSR1::FLI1 expression and function. Where applicable: the error bars indicate SEM; the P values were calculated using one-way ANOVA; *P<0.05; **P<0.01; ***P<0.001; and ****P<0.0001.

FIG. 4A is an immunoblot showing EWSR1::FLI1 levels (monitored using an anti-FLI1 antibody) in two independent C1GALT1 knockout (KO) A673 clones.

FIG. 4B. Left, Immunoblot showing EWSR1::FLI1 levels (monitored using an anti-FLI1 antibody) in A673 cells expressing one of two independent shRNAs targeting C1GALT1C1. Right, qRT-PCR analysis monitoring knockdown efficiencies of the C1GALT1C1 shRNAs.

FIGS. 4C-4F are qRT-PCR analyses monitoring expression of EWSR1::FLI1 target genes in A673 cells following shRNA-mediated knockdown of EWSR1 or C1GALT1.

FIGS. 5A-5F show that the C1GALT1 inhibitor itraconazole (ITZ) reduces EWSR1::FLI1 levels and function. Where applicable: the error bars indicate SEM; the P values were calculated using one-way ANOVA; *P<0.05; **P<0.01; ***P<0.001; and ****P<0.0001.

FIGS. 5A-5B show EWSR1::FLI1 expression (qRT-PCR, FIG. 5A) or EWSR1::FLI1 levels (immunoblot monitored using an anti-FLI1 antibody, FIG. 5B) in A673 cells treated with ITZ (0, 20, 40, 60, 80, 100 nM) for 72 hours.

FIG. 5C is an immunoblot showing the absence of detectable cytochrome P450 3A4 (CYP3A4) in human ES cell lines A673, TC-71, TC-32, and TC-106. HepG2 human liver cells were used as a positive control.

FIG. 5D shows a flow cytometry analysis of tdTomato in A673/EWSR1::FLI1^{tdTomato}/Cas9/EGFP cells treated with 100 nM ITZ, 10 μM ketoconazole (KETO; a CYP3A4 inhibitor), or DMSO for 72 hours. The results show that ketoconazole does not reduce expression of the EWSR1::FLI1^{tdTomato} reporter.

FIGS. 5E-5F are qRT-PCR analyses monitoring expression of EWSR1::FLI1 target genes in A673 cells following treatment with ITZ (100 nM for 72 hours).

FIGS. 6A-6G show that EWSR1::FLI1 expression is promoted by Hedgehog (Hh) signaling. Where applicable: error bars indicate SEM; P values were calculated using an unpaired, two-tailed t-test (FIG. 6A) or one-way ANOVA (FIGS. 6B-6F); *P<0.05; **P<0.01; ***P<0.001; and ****P<0.0001.

FIG. 6A shows immunoblots (top) and qRT-PCR analyses (bottom) monitoring EWSR1::FLI1 protein (using an anti-FLI1 antibody) and mRNA levels, respectively, in A673 cells following siRNA-mediated knockdown of SMO (left), GLI1 (middle), or GLI2 (right) using two independent siRNAs.

FIG. 6B shows immunoblots (top) and qRT-PCR analyses (bottom) monitoring EWSR1::FLI1 protein (using an anti-FLI1 antibody) and mRNA levels, respectively, following treatment of A673 cells with cyclopamine (0, 2.5, 5, 7.5 μM for 48 hours) or GANT61 (0, 5, 7.5, 10 μM for 48 hours).

FIG. 6C shows an immunoblot (top) and qRT-PCR analysis (bottom) monitoring EWSR1::FLI1 protein (using an anti-FLI1 antibody) and mRNA levels, respectively, following treatment of A673 cells with Smoothened Agonist hydrochloride (SAG) (0, 2.5, 5, 7.5, 10 μM for 48 hours).

FIG. 6D is a schematic illustration of a GLI consensus sequence and locations and sequences of candidate GLI-binding sites in EWSR1. P1 is located at positions −4555 to −4548 relative to the transcription start site; P2 is located at positions −655 to −648; P3 is located at positions −446 to −439; and 12 (12-1) is located at positions 4085 to 4094.

FIG. 6E shows chromatin immunoprecipitation (ChIP) analysis in A673 cells monitoring binding of GLI1, in the presence or absence of ITZ (100 nM for 72 hours), to EWSR1 regions containing GLI-binding motifs (P1, P2, P3, 12-1) or, as a negative control, a gene desert region (Control). The results were normalized to that obtained with IgG, which was set to 1.

FIGS. 6F-6G show EWSR1::FLI1 expression (qRT-PCR, FIG. 6F) or EWSR1::FLI1 levels (immunoblot monitored using an anti-FLI1 antibody, FIG. 6G) in A673 cells in which the GLI-binding sites P2 and P3 were deleted.

FIGS. 7A-7L show that C1GALT1 stimulates Hh signaling by promoting O—N-acetylgalactosamine-glycosylation (O-GalNAc-glycosylation) and stabilization of SMO. Where applicable: the error bars indicate SEM; the P values were calculated using one-way ANOVA; *P<0.05; **P<0.01; ***P<0.001; and ****P<0.0001.

FIGS. 7A-7B show immunoblot (left) and qRT-PCR (right) analyses monitoring expression of SMO and GLI1 in A673 cells following knockdown of C1GALT1 using two independent shRNAs (FIG. 7A) or treatment with ITZ (100 nM for 72 hours) (FIG. 7B).

FIG. 7C. Results from a GLI-driven luciferase reporter assay showing relative luciferase activity in A673 cells transfected with a luciferase reporter plasmid driven by 10 GLI-binding sites and expressing an NS, C1GALT1, or SMO shRNA.

FIG. 7D. Immunoblot monitoring C1GALT1 levels in NIH 3T3 cells expressing an empty vector or C1GALT1-expression plasmid. The results confirmed elevated levels of C1GALT1 in cells expressing the C1GALT1-expression plasmid.

FIG. 7E. Immunoblot (left) and qRT-PCR analysis (right) monitoring expression of SMO and GLI1 in NIH 3T3 cells following treatment with SAG or overexpressing C1GALT1.

FIG. 7F. GLI-driven luciferase reporter assay (left) in NIH 3T3 cells expressing empty vector, C1GALT1, or SMO; and an immunoblot confirming elevated expression of SMO (right).

FIG. 7G. Left, schematic representation of the peanut agglutinin (PNA) lectin pull-down assay, illustrating binding of a PNA lectin bead to T antigen (a disaccharide comprising GalNAc (black square) and galactosamine (Gal; black circle), which is attached to SMO through a serine or threonine (S/T) residue. The site of O-glycosidase (O-glyc) cleavage is shown. Right, Immunoblots showing levels of O-glycosylated SMO (as monitored by a PNA lectin pull-down assay) and total SMO (from whole cell extracts) following treatment of A673 cell extracts with a combination of O-glycosidase and α2-3,6,8 neuraminidase (+), which removes terminal sialic residues that can inhibit binding to lectins and cleavage by O-glycosidase.

FIG. 7H. Co-immunoprecipitation assay in A673 cells monitoring the presence of SMO in a C1GALT1 immunoprecipitate (IP) or vice versa.

FIG. 7I. Immunoblot analysis monitoring levels of O-glycosylated SMO (as monitored by a PNA lectin pull-down assay) as well as total SMO and C1GALT1 in A673 cells treated with 100 nM ITZ for 0, 1, 4, 8, 12, 24, 36, or 48 hours.

FIG. 7J. Cycloheximide chase assay. Immunoblot showing SMO levels in A673 cells treated with cycloheximide in the presence or absence of 100 nM ITZ (top). Quantification of SMO protein levels, time 0 in the presence of DMSO was set to 100% (bottom).

FIG. 7K. Immunoblot showing SMO levels in A673 cells treated with DMSO or treated with 100 nM ITZ for 72 hours in the presence or absence of MG132 or BafA1.

FIG. 7L. Immunoblot showing FLAG-SMO levels in A673 cells stably expressing FLAG-tagged derivatives of wild-type SMO, SMO (T55A) or SMO (T500A) mutant in the presence or absence of MG132 or BafA1.

FIG. 7M. Ubiquitination immunoprecipitation (IP) assay showing ubiquitination of FLAG-SMO (T55A) and FLAG-SMO (T500A) mutants in A673 cells expressing HA-tagged ubiquitin (HA-Ub) and treated in the presence or absence of MG132 or BafA1. Also shown is an immunoblot (IB) showing levels of FLAG-SMO, FLAG-SMO (T55A), and FLAG-SMO (T500A) in cell extracts.

FIGS. 8A-8H show that C1GALT1 is required for EWSR1::FLI1-mediated cellular transformation. Error bars indicate SEM (FIGS. 8A, 8E, 8F) or SD (FIGS. 8B, 8G). When applicable: the P values were calculated using one-way ANOVA; *P<0.05; **P<0.01; and ****P<0.0001.

FIG. 8A. Soft agar assay showing colony formation of A673 cells expressing an NS, EWSR1, C1GALT1, or SMO shRNA. Left, representative images. Scale bars correspond to 20 μm. Right, quantification of the number of colonies.

FIG. 8B. Tumor formation. A673 cells expressing an NS, EWSR1, C1GALT1, or SMO shRNA (n=3 mice per group) were injected subcutaneously into NOD scid gamma (NSG) mice, and tumor dimensions was measured every 3 days for 18 days.

FIG. 8C. Immunoblot analysis confirming elevated levels of EWSR1::FLI1 in NIH 3T3 cells transduced with a plasmid expressing EWSR1::FLI1.

FIG. 8D. Soft agar assay showing colony formation of NIH 3T3 cells expressing EWSR1::FLI1 or empty vector. Scale bars correspond to 20 μm.

FIG. 8E. Soft agar assay quantifying colony formation of NIH 3T3 cells expressing EWSR1::FLI1 and an NS, C1GALT1 or SMO shRNA.

FIG. 8F. Soft agar assay showing colony formation of NIH 3T3 cells expressing SMO or C1GALT1. Top, representative images. Scale bars correspond to 20 μm. Bottom, quantification of the number of colonies.

FIG. 8G. Tumor formation. NIH 3T3 cells expressing vector, C1GALT1 or SMO were injected subcutaneously into mice, and tumor dimensions were as measured every 5 days.

FIG. 8H. Immunohistochemistry analysis monitoring H&E staining or Ki67 staining in tumors derived from NIH 3T3 cells ectopically expressing C1GALT1 or SMO. Scale bars correspond to 130 μm.

FIGS. 9A-9F show that C1GALT1 promotes Hh signaling, EWSR1::FLI1 expression, and EWSR1::ERG expression in additional human ES cell lines. Where applicable: error bars indicate SEM; the P values were calculated using one-way ANOVA followed by Dunnett's multiple comparisons test; *P<0.05; **P<0.01; and ***P<0.001.

FIGS. 9A-9B. Immunoblots showing EWSR1::FLI1 protein levels (monitored using an anti-FLI1 antibody) in TC-71 and TC-32 cells expressing a control siRNA or one of two independent SMO (FIG. 9A) or GLI1 (FIG. 9B) siRNAs.

FIG. 9C. Immunoblot showing EWSR1::ERG protein levels (monitored using an anti-EWSR1 antibody) in TC-106 cells expressing a SMO (left) or GLI1 (right) siRNA.

FIG. 9D. Top, qRT-PCR analyses monitoring expression of EWSR1::FLI1 in TC-71 and TC-32 cells, and EWSR1::ERG in TC10⁶ cells, following treatment with DMSO or ITZ for 48 hours. Bottom, Immunoblots showing EWSR1::FLI1 protein levels (monitored using an anti-FLI1 antibody) in TC-71 and TC-32 cells, or EWSR1::ERG protein levels (monitored using an anti-EWSR1 antibody) in TC-106 cells, as well as C1GALT1 and SMO following treatment with DMSO or ITZ for 48 hours.

FIG. 9E. Immunoblot analysis monitoring levels of O-glycosylated SMO (as monitored by a PNA lectin pull-down assay) in TC-71 or TC-32 cells treated with 100 nM ITZ for 48 hours.

FIG. 9F. Immunoblot showing FLAG-SMO levels in TC-71 cells stably expressing FLAG-tagged derivatives of wild-type SMO, SMO (T55A) or SMO (T500A) mutant in the presence or absence of MG132 or BafA1.

FIGS. 10A-10F show that ITZ inhibits proliferation of human ES cell lines in culture. Where applicable: the error bars indicate SD; the P values were calculated using one-way ANOVA (FIGS. 10A, 10C, 10E) or paired t-test (FIG. 10F); *P<0.05; and **P<0.01.

FIG. 10A. Cell viability, as monitored by PRESTOBLUE® assay, of TC-71 cells treated with ITZ (0, 20, 40, 60, 80 and 100 nM for 72 hours).

FIG. 10B. Immunoblot monitoring levels of cleaved PARP in TC-71 cells treated with ITZ (0, 20, 40, 60 and 80 nM for 48 hours). The results show cleavage of PARP (indicated by the arrow), indicative of apoptosis.

FIG. 10C. Cell viability, as monitored by PRESTOBLUE® assay, of TC-32 cells treated with ITZ (0, 20, 40, 60, 80 and 100 nM for 72 hours).

FIG. 10D. Immunoblot monitoring levels of cleaved PARP in TC-32 cells treated with ITZ (0, 20, 40, 60 and 80 nM for 48 hours).

FIG. 10E. Cell viability, as monitored by PRESTOBLUE® assay, of TC-71 cells treated with sonidegib (left) or vismodegib (right) for 72 hours.

FIG. 10F. Cell viability assay for TC-71 cells expressing vector or EWSR1::FLI1 and treated with ITZ for 72 hours.

FIGS. 11A-11I show that ITZ suppresses growth of ES xenografts in mice. Where applicable: the error bars indicate SD; the P values were calculated using an unpaired, two-tailed t-test; **P<0.01.

FIG. 11A. Tumor formation. TC-71 cells were injected subcutaneously into NSG mice (n=3). When tumors reached about 100 mm³, the mice were treated with ITZ (100 mg per kg body weight (mg/kg) daily by oral gavage), and the tumor dimensions were monitored every 3 days for 18 days.

FIG. 11B. Immunohistochemistry images showing staining for Ki67, FLI1, C1GALT1, SMO and GLI1 in TC-71 xenografts from vehicle- or ITZ-treated mice following 18 days of treatment. Scale bars correspond to 20 μm.

FIGS. 11C-11D. Tumor formation (FIG. 11C) and immunohistochemistry analysis (FIG. 11D) of TC-32 xenografts, as described in FIGS. 11A-11B.

FIGS. 11E-11F. Tumor formation (FIG. 11E) and immunohistochemistry analysis (FIG. 11F) of TC-106 xenografts, as described in FIGS. 11A-11B.

FIG. 11G. Animal weight of mice injected with TC-71, TC-32 or TC-106 ES cell lines and treated with vehicle or ITZ. The results show that the dose of ITZ used in these experiments is well tolerated.

FIG. 11H. Tumor formation. TC-71 cells expressing vector or EWSR1::FLI1 were injected subcutaneously into mice. When tumors reached about 100 mm³, mice were treated with vehicle or ITZ as described in FIG. 11A.

FIG. 11I. Tumor formation. Mice were subcutaneously injected with TC-71 cells and treated with ITZ for 18 days. After 18 days, drug (ITZ) treatment was stopped and tumor growth was monitored for another 15 days.

FIG. 12. A model for C1GALT1-mediated regulation of Hh signaling and EWSR1::FLI1 expression.

FIGS. 13A-13C show that biological inhibition of C1GALT1 reduces Hh Signaling in human ovarian (OVCAR8 and SKOV3) and pancreatic (HPAFII and PANC1) cancer cell lines. Where applicable: the error bars indicate SEM; the P values were calculated using an unpaired, two-tailed t-test; **P<0.01; ***P<0.001; and ****P<0.0001.

FIG. 13A. qRT-PCR analysis monitoring expression of SMO and GLI1 in ovarian or pancreatic cancer cell lines expressing an NS or C1GALT1 shRNA.

FIG. 13B. Immunoblot monitoring SMO and GLI1 protein levels in ovarian or pancreatic cancer cell lines expressing an NS or C1GALT1 shRNA.

FIG. 13C. qRT-PCR analysis monitoring knockdown efficiency of the C1GALT1 shRNA in ovarian and pancreatic cancer cell lines.

### DETAILED DESCRIPTION

A description of example embodiments follows.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting.

When introducing elements disclosed herein, the articles “a,” “an,” “the,” and “said” are intended to mean that there are one or more of the elements. Further, the one or more elements may be the same or different.

Throughout this specification and the claims which follow, unless the context requires otherwise, the term “comprise,” and variations such as “comprises” and “comprising”, will be understood to imply the inclusion of, e.g., a stated integer or step or group of integers or steps, but not the exclusion of any other integer or step or group of integer or step. When used herein, the term “comprising” can be substituted with the term “containing” or “including.”

As used herein, the term “consisting of” excludes any element, step, or ingredient not specified in the claim element. When used herein, the term “consisting essentially of” does not exclude materials or steps that do not materially affect the basic and novel characteristics of the claim.

Any of the terms “comprising,” “containing,” “including,” and “having,” whenever used herein in the context of an aspect or embodiment disclosed herein, can in some embodiments, be replaced with the term “consisting of,” or “consisting essentially of” to vary scopes disclosed herein.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

It should be understood that for all numerical bounds describing some parameter in this application, such as “about,” “at least,” “less than,” “fewer than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used herein, the term “about” means within an acceptable error range for a particular value, as determined by one of ordinary skill in the art. Typically, an acceptable error range for a particular value depends, at least in part, on how the value is measured or determined, e.g., the limitations of the measurement system. For example, “about” can mean within an acceptable standard deviation, per the practice in the art. Alternatively, “about” can mean a range of +20%, e.g., +10%, +5%, or =1% of a given value. It is to be understood that the term “about” can precede any particular value specified herein, except for particular values used in the Exemplification. When “about” precedes a range, as in “about 0.0001-100 mg,” the term “about” should be read as applying to both given values of the range, such that “about 0.0001-100 mg” means about 0.0001 mg to about 100 mg.

Compounds described herein include those described generally, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the relevant contents of which are incorporated herein by reference.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by reference herein for its chemical structure names and rules on naming chemical structures. Optionally, a name of a compound may be generated using a chemical naming program (e.g., CHEMDRAW®, version 17.0.0.206, PerkinElmer Informatics, Inc.).

“Alkyl” refers to a branched or straight-chain, monovalent, hydrocarbon radical having the specified number of carbon atoms. Thus, “(C2-C₈)alkyl” refers to a radical having from 2-8 carbon atoms in a branched or linear arrangement. Typically, alkyl is (C₁-C₂₅)alkyl, e.g., (C₁-C₁₅)alkyl, (C₁-C₁₀)alkyl, (C₁-C₈)alkyl, (C₂-C₈)alkyl, (C₁-C₆)alkyl, (C₂-C₆)alkyl, (C₁-C₅)alkyl, (C₂-C₅)alkyl or (C₂-C₃)alkyl. Examples of alkyl groups include methyl, ethyl, n-propyl, isopropyl, n-butyl, isobutyl, sec-butyl, t-butyl, n-pentyl, isopentyl, neopentyl, 2-methylpentyl, n-hexyl, and the like. In some embodiments, alkyl is optionally substituted, e.g., with one or more substituents described herein.

“Aryl” refers to a monocyclic or polycyclic (e.g., bicyclic, tricyclic), aromatic, hydrocarbon ring system having the specified number of ring atoms, and includes aromatic rings fused to non-aromatic rings, as long as one of the fused rings is an aromatic hydrocarbon. Thus, “(C₆-C₁₅) aryl” refers to a ring system having from 6-15 ring atoms. Examples of aryl include phenyl, naphthyl and fluorenyl. In some embodiments, aryl (e.g., (C₆-C₁₅) aryl) is phenyl, naphthyl or fluorenyl. In some embodiments, aryl is optionally substituted, e.g., with one or more substituents described herein.

“Heteroaryl” refers to a monocyclic or polycyclic (e.g., bicyclic, tricyclic), aromatic, hydrocarbon ring system having the specified number of ring atoms, wherein at least one carbon atom in the ring system has been replaced with a heteroatom selected from nitrogen, sulfur and oxygen. Thus, “(C₅-C₁₅) heteroaryl” refers to a heteroaromatic ring system having from 5-15 ring atoms consisting of carbon, nitrogen, sulfur and oxygen. “Heteroaryl” includes heteroaromatic rings fused to non-aromatic rings, as long as one of the fused rings is a heteroaromatic hydrocarbon. A heteroaryl can contain 1, 2, 3 or 4 (e.g., 1, 2 or 3) heteroatoms independently selected from nitrogen, sulfur and oxygen. In some embodiments, a heteroaryl contains 1, 2 or 3 heteroatoms, each of which is nitrogen. Typically, heteroaryl is (C₅-C₂₀) heteroaryl, e.g., (C₅-C₁₅) heteroaryl, (C₅-C₁₂) heteroaryl, C₅ heteroaryl or C₆ heteroaryl. Monocyclic heteroaryls include, but are not limited to, furan, oxazole, thiophene, triazole, triazene, thiadiazole, oxadiazole, imidazole, isothiazole, isoxazole, pyrazole, pyridazine, pyridine, pyrazine, pyrimidine, pyrrole, tetrazole and thiazole. Bicyclic heteroaryls include, but are not limited to, indolizine, indole, isoindole, indazole, benzimidazole, benzofuran, benzothiazole, purine, quinoline, isoquinoline, cinnoline, phthalazine, quinazoline, quinoxaline, naphthyridine and pteridine. In some embodiments, heteroaryl (e.g., (C₅-C₁₅) heteroaryl) is pyridinyl, pyrimidinyl or carbazolyl. In some embodiments, heteroaryl is optionally substituted, e.g., with one or more substituents described herein.

“Alkoxy” refers to an alkyl radical attached through an oxygen linking atom, wherein alkyl is as described herein. Examples of alkoxy include, but are not limited to, methoxy, ethoxy, propoxy, isopropoxy, and the like.

“Halogen” and “halo” are used interchangeably herein and each refers to fluorine, chlorine, bromine, or iodine. In some embodiments, halo is fluoro, chloro or bromo. In some embodiments, halo is fluoro.

Unless otherwise stated, structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this disclosure. In all provided structures, any hydrogen atom can also be independently selected from deuterium (²H), tritium (³H) and/or fluorine (¹⁸F). Such compounds are useful, for example, as analytical tools, as probes in biological assays, or as therapeutic agents in accordance with the present disclosure.

The terms “derived from” and “derivative of,” as used herein, refer to a chemical structure that is homologous to or structurally similar to a related chemical structure.

The phrase “pharmaceutically acceptable” means that the substance or composition the phrase modifies is, within the scope of sound medical judgment, suitable for use in contact with the tissues of humans and lower animals without undue toxicity, irritation, allergic response, and the like, and are commensurate with a reasonable benefit/risk ratio.

As used herein, the term “pharmaceutically acceptable salt” refers to those salts which are, within the scope of sound medical judgment, suitable for use in contact with the tissues of mammals without undue toxicity, irritation, allergic response, and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety. Pharmaceutically acceptable salts of the agents/compounds described herein include salts derived from suitable inorganic and organic acids, and suitable inorganic and organic bases.

Examples of salts derived from suitable acids include salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid or by using other methods used in the art, such as ion exchange. Other pharmaceutically acceptable salts derived from suitable acids include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like.

Either the mono-, di- or tri-acid salts can be formed, and such salts can exist in either a hydrated, solvated or substantially anhydrous form.

Salts derived from appropriate bases include salts derived from inorganic bases, such as alkali metal, alkaline earth metal, and ammonium bases, and salts derived from aliphatic, alicyclic or aromatic organic amines, such as methylamine, trimethylamine and picoline, or N⁺((C₁-C₄)alkyl)₄ salts. Representative alkali or alkaline earth metal salts include sodium, lithium, potassium, calcium, magnesium, barium and the like. Further pharmaceutically acceptable salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

Compounds described herein can also exist as “solvates” or “hydrates.” A “hydrate” is a compound that exists in a composition with one or more water molecules. A hydrate can include water in stoichiometric quantities, such as a monohydrate or a dihydrate, or can include water in random amounts. A “solvate” is similar to a hydrate, except that a solvent other than water, such as methanol, ethanol, dimethylformamide, diethyl ether, or the like replaces water. Mixtures of such solvates or hydrates can also be prepared. The source of such solvate or hydrate can be from the solvent of crystallization, inherent in the solvent of preparation or crystallization, or adventitious to such solvent.

Compounds disclosed herein may exist as stereoisomers. For example, compounds disclosed herein may have asymmetric centers, chiral axes, and chiral planes (e.g., as described in: E. L. Eliel and S. H. Wilen, Stereo-chemistry of Carbon Compounds, John Wiley & Sons, New York, 1994, pages 1119-1190), and occur as racemates, racemic mixtures, or as individual diastereomers or enantiomers.

Unless otherwise indicated, all possible isomers and mixtures thereof, including optical isomers, rotamers, tautomers and cis- and trans-isomers, are included in the present invention.

When a disclosed compound is depicted by structure without indicating the stereochemistry, and the compound has one chiral center, it is to be understood that the structure encompasses one enantiomer or diastereomer of the compound separated or substantially separated from the corresponding optical isomer(s), a racemic mixture of the compound and mixtures enriched in one enantiomer or diastereomer relative to its corresponding optical isomer(s).

When a disclosed compound is depicted by a structure indicating stereochemistry, and the compound has more than one chiral center, the stereochemistry indicates relative stereochemistry, rather than the absolute configuration of the substituents around the one or more chiral carbon atoms. “R” and “S” are used to indicate the absolute configuration of substituents around one or more chiral carbon atoms.

“Enantiomers” are pairs of stereoisomers that are non-superimposable mirror images of one another, most commonly because they contain an asymmetrically substituted carbon atom that acts as a chiral center.

“Diastereomers” are stereoisomers that are not related as mirror images, most commonly because they contain two or more asymmetrically substituted carbon atoms.

“Racemate” or “racemic mixture,” as used herein, refer to a mixture containing equimolar quantities of two enantiomers of a compound. Such mixtures exhibit no optical activity (i.e., they do not rotate a plane of polarized light).

Methods of obtaining an optical isomer separated or substantially separated from the corresponding optical isomer(s) are known in the art. For example, an optical isomer can be purified from a racemic mixture by well-known chiral separation techniques, such as, but not limited to, normal- and reverse-phase chromatography, and crystallization. An optical isomer can also be prepared by the use of chiral intermediates or catalysts in synthesis. In some cases, compounds having at least some degree of enantiomeric enrichment can be obtained by physical processes, such as selective crystallization of salts or complexes formed with chiral adjuvants.

As used herein, the term “compound of the disclosure” refers to a compound of any structural formula depicted herein (e.g., a compound of structural formula I or a subformula thereof)), as well as isomers, such as stereoisomers (including diastereoisomers, enantiomers and racemates) and tautomers thereof, isotopologues thereof, and inherently formed moieties (e.g., polymorphs and/or solvates, such as hydrates) thereof. When a moiety is present that is capable of forming a salt, then salts are included as well, in particular, pharmaceutically acceptable salts.

“Pharmaceutically acceptable carrier” refers to a non-toxic carrier or excipient that does not destroy the pharmacological activity of the agent with which it is formulated and is nontoxic when administered in doses sufficient to deliver a therapeutic amount of the agent. Pharmaceutically acceptable carriers that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

“Treating” or “treatment,” as used herein, refers to taking steps to deliver a therapy to a subject, such as a mammal, in need thereof (e.g., as by administering to a mammal one or more therapeutic agents). “Treating” or “treatment” includes inhibiting the disease or condition (e.g., as by slowing or stopping its progression or causing regression of the disease or condition) and/or relieving the symptoms resulting from the disease or condition. As used herein, the term “treating,” or “treatment” refers to the medical management of a subject with the intent to improve, ameliorate, stabilize (i.e., not worsen), prevent or cure a disease or condition (such as Ewing sarcoma). This term includes active treatment (treatment directed to improve the disease or condition), causal treatment (treatment directed to the cause of the associated disease or condition), palliative treatment (treatment designed for the relief of symptoms), preventative (e.g., prophylactic) treatment (treatment directed to minimizing or partially or completely inhibiting the development of the associated disease or condition); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the disease or condition, as well as those prone to have the disease or condition or those in which the disease or condition is to be prevented.

“Administering” or “administration,” as used herein, refers to providing a compound, composition, or pharmaceutically acceptable salt thereof described herein to a subject in need of treatment or prevention.

“A therapeutically effective amount” or “an effective amount” refers to an amount effective, at dosages and for periods of time necessary, to achieve a desired therapeutic or biological result (e.g., treatment, healing, inhibition or amelioration of physiological response or condition, etc.). Non-limiting examples of desired therapeutic or biological results include reduced tumor size, improved radiological response, prolonged event-free survival, prolonged disease-free survival, prolonged metastasis-free survival, prolonged overall survival, or any combination of the foregoing.

The full therapeutic effect does not necessarily occur by administration of one dose and may occur only after administration of a series of doses. Thus, a therapeutically effective amount may be administered in one or more administrations. A therapeutically effective amount may vary according to factors such as disease state, age, sex, and weight of an individual, e.g., a mammal, mode of administration and the ability of a therapeutic, or combination of therapeutics, to elicit a desired response in an individual.

An effective amount of an agent to be administered can be determined by a clinician of ordinary skill using the guidance provided herein and other methods known in the art. For example, suitable dosages can be from about 0.001 mg/kg to about 100 mg/kg, from about 0.01 mg/kg to about 100 mg/kg, from about 0.01 mg/kg to about 10 mg/kg, or from about 0.01 mg/kg to about 1 mg/kg body weight per treatment. Determining the dosage for a particular agent, subject and disease is well within the abilities of one of skill in the art. Preferably, the dosage does not cause or produces minimal adverse side effects.

As used herein, the terms “subject” and “patient” are used herein interchangeably to refer to an animal (e.g., a mammal, such as a human) that is to be treated according to a method disclosed herein. A subject to be treated according to methods described herein may be one that has been diagnosed with a particular disease or condition (e.g., Ewing sarcoma), or one at risk of developing such disease or condition. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

Headings used in this application are for convenience only and do not affect the interpretation of this application.

Preferred features of each of the aspects or embodiments provided by the invention are applicable to all of the other aspects or embodiments of the invention mutatis mutandis and, without limitation, are exemplified by the dependent claims and also encompass combinations and permutations of individual features (e.g., elements, including numerical ranges and exemplary embodiments) of particular embodiments and aspects of the invention, including the working examples. For example, particular experimental parameters exemplified in the working examples can be adapted for use in the claimed invention piecemeal without departing from the invention. For example, for materials that are disclosed, while specific reference of each of the various individual and collective combinations and permutations of these compounds may not be explicitly disclosed, each is specifically contemplated and described herein. Thus, if a class of elements A, B, and C are disclosed as well as a class of elements D, E, and F and an example of a combination of elements A-D is disclosed, then, even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-groups of A-E, B-F, and C-E are specifically contemplated and should be considered disclosed from disclosure of A, B, and C; D, E, and F; and the example combination A-D. This concept applies to all aspects of this application, including elements of a composition of matter and steps of method of making or using the compositions.

The forgoing aspects of the invention, as recognized by the person having ordinary skill in the art following the teachings of the specification, can be claimed in any combination or permutation to the extent that they are novel and non-obvious over the prior art-thus, to the extent an element is described in one or more references known to the person having ordinary skill in the art, they may be excluded from the claimed invention by, inter alia, a negative proviso or disclaimer of the feature or combination of features.

### Methods of Treating Ewing Sarcoma

Provided herein, among other things, are methods of treating Ewing sarcoma in a subject, the methods comprising administering to the subject an effective amount of an agent or a pharmaceutically acceptable salt thereof that:
a) reduces expression of core 1 synthase, glycoprotein-N-acetylgalactosamine 3-beta-galactosyltransferase 1 (C1GALT1) (e.g., in the subject);
b) reduces activity of C1GALT1 (e.g., in the subject);
c) or both a) and b), thereby treating Ewing sarcoma in the subject.

Also provided herein, among other things, are methods of treating Ewing sarcoma in a subject, comprising administering to the subject an effective amount of an agent that reduces expression and/or activity of C1GALT1, or a pharmaceutically acceptable salt thereof, thereby treating Ewing sarcoma in the subject.

### Subjects

In some embodiments, a subject is a mammal. As used herein, the term “subject” includes humans, domestic animals, such as laboratory animals (e.g., dogs, monkeys, pigs, rats, mice, etc.), household pets (e.g., cats, dogs, rabbits, etc.), livestock (e.g., pigs, cattle, sheep, goats, horses, etc.), and non-domestic animals. In some embodiments, a subject is a human. In some embodiments, a subject (e.g., human) is male. In some embodiments a subject (e.g., human) is female. In some embodiments, a subject (e.g., human) is intersex. In some embodiments, a subject (e.g., human): has physiological and/or genetic characteristics associated with one or more sexes; has undergone or received medical interventions that affect physiological characteristics associated with one or more sexes; and/or is intersex. In some embodiments, the sex of a subject is undefined, unknown, or unclear.

In some embodiments, a subject (e.g., a human) has cancer (e.g., sarcoma such as Ewing sarcoma). In some embodiments, a subject is diagnosed with cancer. In some embodiments, a subject is suspected of having cancer. In some embodiments, a subject is prone to have cancer.

In some embodiments, cancer is Ewing sarcoma, pancreatic cancer, ovarian cancer, liver cancer, gastric cancer, prostate cancer, esophageal cancer, or head and neck cancer. In some embodiments, cancer is Ewing sarcoma, ovarian cancer, or pancreatic cancer. In some embodiments, cancer is Ewing sarcoma (also referred to as Ewing's sarcoma family of tumors, Ewing sarcoma family of tumors, or Ewing family of tumors). In some embodiments, cancer is pancreatic cancer. In some embodiments, cancer is ovarian cancer. In some embodiments, cancer is liver cancer. In some embodiments, cancer is gastric cancer. In some embodiments, cancer is prostate cancer. In some embodiments, cancer is esophageal cancer. In some embodiments, cancer is head and neck cancer.

In some embodiments, cancer is Ewing sarcoma. In some embodiments, Ewing sarcoma is Ewing sarcoma of bone, extraosseous Ewing sarcoma (EOE), peripheral primitive neuroectodermal tumor (pPNET), or Askin tumor.

In some embodiments, Ewing sarcoma (e.g., Ewing sarcoma of bone, extraosseous Ewing sarcoma (EOE), peripheral primitive neuroectodermal tumor (pPNET), or Askin tumor) is non-metastatic, localized, metastatic, advanced, or relapsed or refractory. In some embodiments, Ewing sarcoma is localized. In some embodiments, Ewing sarcoma is metastatic. In some embodiments, Ewing sarcoma is advanced Ewing sarcoma. In some embodiments, Ewing sarcoma is relapsed or refractory. In some embodiments, refractory Ewing sarcoma is first recurrent extracranial Ewing sarcoma.

In some embodiments, a subject (e.g., a human) is newly diagnosed with Ewing sarcoma, for example, newly diagnosed with non-metastatic Ewing sarcoma, localized Ewing sarcoma, or metastatic Ewing sarcoma. In some embodiments, a subject is newly diagnosed with non-metastatic Ewing sarcoma. In some embodiments, a subject is newly diagnosed with localized Ewing sarcoma. In some embodiments, a subject is newly diagnosed with metastatic Ewing sarcoma.

In some embodiments, a subject (e.g., a human) has or is susceptible to metastatic Ewing sarcoma, relapsed or recurrent Ewing sarcoma, non-metastatic Ewing sarcoma, or localized Ewing sarcoma. In some embodiments, a subject has or is susceptible to metastatic Ewing sarcoma. In some embodiments, a subject has or is susceptible to relapsed or recurrent Ewing sarcoma. In some embodiments, a subject has or is susceptible to non-metastatic Ewing sarcoma. In some embodiments, a subject has or is susceptible to localized Ewing sarcoma.

In some embodiments, a subject has metastatic Ewing sarcoma, relapsed or recurrent Ewing sarcoma, non-metastatic Ewing sarcoma, or localized Ewing sarcoma. In some embodiments, a subject has metastatic Ewing sarcoma. In some embodiments, a subject has relapsed or recurrent Ewing sarcoma. In some embodiments, a subject has non-metastatic Ewing sarcoma. In some embodiments, a subject has localized Ewing sarcoma.

In some embodiments, a subject is susceptible to metastatic Ewing sarcoma, relapsed or recurrent Ewing sarcoma, non-metastatic Ewing sarcoma, or localized Ewing sarcoma. In some embodiments, a subject is susceptible to metastatic Ewing sarcoma. In some embodiments, a subject is susceptible to relapsed or recurrent Ewing sarcoma. In some embodiments, a subject is susceptible to non-metastatic Ewing sarcoma. In some embodiments, a subject is susceptible to localized Ewing sarcoma.

In some embodiments, a subject (e.g., a human) is a high-risk Ewing sarcoma patient.

In some embodiments, a subject (e.g., a human) has one or more oncogenic mutations (e.g., substitution, insertion, deletion), chromosomal rearrangements (e.g., duplication, deletion, inversion, or translocation), and/or aberrations (e.g., aberrant expression of tumorigenic or pro-tumor factors, inactivation of tumor suppressor expression). In some embodiments, a subject (e.g., a human) has one or more oncogenic fusions. In some embodiments, a subject (e.g., a human) has one or more fusion genes. In some embodiments, a fusion gene is an oncogenic fusion gene. In some embodiments, a subject (e.g., a human) has one or more oncogenic fusion genes.

In some embodiments, a subject (e.g., a human) has an oncogenic fusion involving EWS RNA binding protein 1 (EWSR1). In some embodiments, a subject (e.g., a human) has a fusion gene involving EWSR1. In some embodiments, a fusion gene involving EWSR1 is expressed in a subject (e.g., a human). In some embodiments, a fusion protein including EWSR1, or a part thereof, is expressed in a subject (e.g., a human).

In some embodiments, a subject (e.g., a human) has EWSR1 and Fli-1 proto-oncogene, ETS transcription factor (FLI1) oncogenic fusion (EWSR1::FLI1, also referred to as EWS-FLI1, EWS::FLI1, or EWSR1-FLI1). In some embodiments, a subject (e.g., a human) has an EWSR1::FLI1 fusion gene. In some embodiments, an EWSR1::FLI1 fusion gene is expressed in a subject (e.g., a human). In some embodiments, an EWSR1::FLI1 fusion protein is expressed in a subject (e.g., a human).

In some embodiments, a subject (e.g., a human) has EWSR1 and ETS transcription factor ERG (ERG) oncogenic fusion (EWSR1::ERG, also referred to as EWS::ERG, EWSR1-ERG, or EWS-ERG). In some embodiments, a subject (e.g., a human) has an EWSR1::ERG fusion gene. In some embodiments, an EWSR1::ERG fusion gene is expressed in a subject (e.g., a human). In some embodiments, an EWSR1::ERG fusion protein is expressed in a subject (e.g., a human).

In some embodiments, a subject (e.g., a human) is a fetus or a neonatal subject. In some embodiments, a subject is a fetus. In some embodiments, a subject is a neonatal subject.

In some embodiments, a subject (e.g., a human) is at least about 1 month of age, for example, at least about: 2, 3, 4, 5, 6, 7, 8, 9, 10, 11, 12, 15, 18, or 21 months of age.

In some embodiments, a subject (e.g., a human) is at least about 1 year of age, for example, at least about: 2, 5, 10, 12, 15, 18, 20, 25, 30, 35, 40, 45, 50, 55, or 60 years of age. In some embodiments, a subject is at least about 18 years of age.

In some embodiments, a subject (e.g., a human) is no more than about 60 years of age, for example, no more than about: 59, 55, 50, 45, 40, 35, 30, 25, 20, 18, 17, 15, 12, 10, 5, 4, 3, or 2 years of age. In some embodiments, a subject is no more than about 50 years of age. In some embodiments, a subject is no more than about 40 years of age.

In some embodiments, a subject (e.g., a human) is about 0-100 years of age, for example, about: 0-80, 0-60, 0-30, 0-24, 0-20, 0-18, 0-17, 0-12, 0-6, 1-100, 1-70, 1-50, 1-40, 1-25, 1-18, 1-15, 1-10, 1-5, 2-100, 2-80, 2-60, 2-30, 2-24, 2-20, 2-18, 5-100, 5-80, 5-60, 5-30, 5-24, 5-20, 5-18, 5-12, 12-100, 12-80, 12-60, 12-50, 12-40, 12-30, 12-24, 18-100, 18-80, 18-75, 18-70, 18-65, 18-60, 18-55, 18-50, 18-45, or 18-40 years of age. In some embodiments, a subject is about 0-60 years of age, e.g., 2-60 years of age. In some embodiments, a subject is about 0-50 years of age, e.g., 2-50 years of age.

In some embodiments, a subject at diagnosis is less than about 50 years of age, for example, a subject at diagnosis is less than about: 45, 40, 35, 30, 25, 20, 18, 17, 15, 12, 10, 8, or 5 years of age, or less than about 1 year of age.

### Agents or Pharmaceutically Acceptable Salts Thereof

In some embodiments, an agent (e.g., ITZ or an analog thereof) or a pharmaceutically acceptable salt thereof reduces expression of C1GALT1 and/or reduces activity of C1GALT1 (e.g., in a subject). In some embodiments, an agent (e.g., ITZ or an analog thereof) or a pharmaceutically acceptable salt thereof reduces expression of C1GALT1 (e.g., in a subject). In some embodiments, an agent (e.g., ITZ or an analog thereof) or a pharmaceutically acceptable salt thereof reduces activity of C1GALT1 (e.g., in a subject). In some embodiments, an agent (e.g., ITZ or an analog thereof) or a pharmaceutically acceptable salt thereof reduces expression of C1GALT1 and reduces activity of C1GALT1 (e.g., in a subject).

In some embodiments, an agent (e.g., ITZ or an analog thereof) or a pharmaceutically acceptable salt thereof:
a) reduces expression of Smoothened (SMO);
b) reduces O—N-acetylgalactosamine-glycosylation (O-GalNAc-glycosylation) of SMO;
c) reduces activity of SMO;
d) reduces stability of SMO;
e) increases ubiquitination of SMO;
f) reduces ligand-independent hedgehog signaling;
g) reduces expression of GLI family zinc finger 1 (GLI1);
h) reduces expression of GLI family zinc finger 2 (GLI2);
i) reduces binding of GLI1 to EWS RNA binding protein 1 (EWSR1);
j) reduces binding of GLI2 to EWSR1;
k) reduces binding of GLI1 to EWSR1 and Fli-1 proto-oncogene, ETS transcription factor (FLI1) fusion gene (EWSR1::FLI1);
l) reduces binding of GLI2 to EWSR1::FLI1;
m) reduces expression of EWSR1::FLI1;
n) reduces expression of an EWSR1-FLI1 fusion protein (EWSR1::FLI1) target gene;
o) reduces binding of GLI1 to EWSR1 and ETS transcription factor ERG (ERG) fusion gene (EWSR1::ERG);
p) reduces binding of GLI2 to EWSR1::ERG;
q) reduces expression of EWSR1::ERG;
r) reduces expression of an EWSR1-ERG fusion protein (EWSR1::ERG) target gene;
or any combination of the foregoing (e.g., in a subject).

In some embodiments, an agent (e.g., ITZ or an analog thereof) or a pharmaceutically acceptable salt thereof reduces expression of SMO (e.g., in a subject).

In some embodiments, an agent (e.g., ITZ or an analog thereof) or a pharmaceutically acceptable salt thereof reduces O-GalNAc-glycosylation of SMO (e.g., in a subject).

In some embodiments, an agent (e.g., ITZ or an analog thereof) or a pharmaceutically acceptable salt thereof reduces activity of SMO (e.g., in a subject).

In some embodiments, an agent (e.g., ITZ or an analog thereof) or a pharmaceutically acceptable salt thereof reduces stability of SMO (e.g., in a subject).

In some embodiments, an agent (e.g., ITZ or an analog thereof) or a pharmaceutically acceptable salt thereof:
a) reduces expression of Smoothened (SMO);
b) reduces O—N-acetylgalactosamine-glycosylation (O-GalNAc-glycosylation) of SMO;
c) reduces activity of SMO;
d) reduces stability of SMO;
or any combination of the foregoing (e.g., in a subject).

In some embodiments, an agent (e.g., ITZ or an analog thereof) or a pharmaceutically acceptable salt thereof increases ubiquitination of SMO (e.g., in a subject).

In some embodiments, an agent (e.g., ITZ or an analog thereof) or a pharmaceutically acceptable salt thereof:
a) reduces expression of Smoothened (SMO);
b) reduces O—N-acetylgalactosamine-glycosylation (O-GalNAc-glycosylation) of SMO;
c) reduces activity of SMO;
d) reduces stability of SMO;
e) increases ubiquitination of SMO;
or any combination of the foregoing (e.g., in a subject).

In some embodiments, an agent (e.g., ITZ or an analog thereof) or a pharmaceutically acceptable salt thereof reduces ligand-independent hedgehog signaling (e.g., in a subject).

In some embodiments, an agent (e.g., ITZ or an analog thereof) or a pharmaceutically acceptable salt thereof reduces expression of GLI1 (e.g., in a subject).

In some embodiments, an agent (e.g., ITZ or an analog thereof) or a pharmaceutically acceptable salt thereof reduces expression of GLI2 (e.g., in a subject).

In some embodiments, an agent (e.g., ITZ or an analog thereof) or a pharmaceutically acceptable salt thereof reduces binding of GLI1 to EWSR1 (e.g., in a subject).

In some embodiments, an agent (e.g., ITZ or an analog thereof) or a pharmaceutically acceptable salt thereof reduces binding of GLI2 to EWSR1 (e.g., in a subject).

In some embodiments, an agent (e.g., ITZ or an analog thereof) or a pharmaceutically acceptable salt thereof reduces expression of EWSR1 (e.g., in a subject).

In some embodiments, an agent (e.g., ITZ or an analog thereof) or a pharmaceutically acceptable salt thereof:
a) reduces expression of GLI1;
b) reduces binding of GLI1 to EWSR1;
c) reduces expression of EWSR1;
or any combination of the foregoing (e.g., in a subject).

In some embodiments, an agent (e.g., ITZ or an analog thereof) or a pharmaceutically acceptable salt thereof:
a) reduces expression of GLI2;
b) reduces binding of GLI2 to EWSR1;
c) reduces expression of EWSR1;
or any combination of the foregoing (e.g., in a subject).

In some embodiments, an agent (e.g., ITZ or an analog thereof) or a pharmaceutically acceptable salt thereof:
a) reduces expression of GLI1;
b) reduces binding of GLI1 to EWSR1;
c) reduces expression of GLI2;
d) reduces binding of GLI2 to EWSR1;
e) reduces expression of EWSR1;
or any combination of the foregoing (e.g., in a subject).

In some embodiments, an agent (e.g., ITZ or an analog thereof) or a pharmaceutically acceptable salt thereof reduces binding of GLI1 to EWSR1::FLI1 (e.g., in a subject).

In some embodiments, an agent (e.g., ITZ or an analog thereof) or a pharmaceutically acceptable salt thereof reduces binding of GLI2 to EWSR1::FLI1 (e.g., in a subject).

In some embodiments, an agent (e.g., ITZ or an analog thereof) or a pharmaceutically acceptable salt thereof reduces expression of EWSR1::FLI1 (e.g., in a subject).

In some embodiments, an agent (e.g., ITZ or an analog thereof) or a pharmaceutically acceptable salt thereof reduces expression of an EWSR1::FLI1 target gene (e.g., in a subject).

In some embodiments, an agent (e.g., ITZ or an analog thereof) or a pharmaceutically acceptable salt thereof:
a) reduces expression of GLI1;
b) reduces binding of GLI1 to EWSR1::FLI1;
c) reduces expression of EWSR1::FLI1;
d) reduces expression of an EWSR1::FLI1 target gene;
or any combination of the foregoing (e.g., in a subject).

In some embodiments, an agent (e.g., ITZ or an analog thereof) or a pharmaceutically acceptable salt thereof:
a) reduces expression of GLI2;
b) reduces binding of GLI2 to EWSR1::FLI1;
c) reduces expression of EWSR1::FLI1;
d) reduces expression of an EWSR1::FLI1 target gene;
or any combination of the foregoing (e.g., in a subject).

In some embodiments, an agent (e.g., ITZ or an analog thereof) or a pharmaceutically acceptable salt thereof:
a) reduces expression of GLI1;
b) reduces binding of GLI1 to EWSR1::FLI1;
c) reduces expression of GLI2;
d) reduces binding of GLI2 to EWSR1::FLI1;
e) reduces expression of EWSR1::FLI1;
f) reduces expression of an EWSR1::FLI1 target gene;
or any combination of the foregoing (e.g., in a subject).

In some embodiments, an agent (e.g., ITZ or an analog thereof) or a pharmaceutically acceptable salt thereof reduces expression of EWSR1::ERG (e.g., in a subject).

In some embodiments, an agent (e.g., ITZ or an analog thereof) or a pharmaceutically acceptable salt thereof:
a) reduces expression of GLI1;
b) reduces binding of GLI1 to EWSR1::ERG;
c) reduces expression of EWSR1::ERG;
d) reduces expression of an EWSR1::ERG target gene;
or any combination of the foregoing (e.g., in a subject).

In some embodiments, an agent (e.g., ITZ or an analog thereof) or a pharmaceutically acceptable salt thereof:
a) reduces expression of GLI2;
b) reduces binding of GLI2 to EWSR1::ERG;
c) reduces expression of EWSR1::ERG;
d) reduces expression of an EWSR1::ERG target gene;
or any combination of the foregoing (e.g., in a subject).

In some embodiments, an agent (e.g., ITZ or an analog thereof) or a pharmaceutically acceptable salt thereof:
a) reduces expression of GLI1;
b) reduces binding of GLI1 to EWSR1::ERG;
c) reduces expression of GLI2;
d) reduces binding of GLI2 to EWSR1::ERG;
e) reduces expression of EWSR1::ERG;
f) reduces expression of an EWSR1::ERG target gene;
or any combination of the foregoing (e.g., in a subject).

In some embodiments, an EWSR1::ERG target gene comprises transforming growth factor beta receptor 2 (TGFBR2), bone morphogenetic protein 4 (BMP4), plasminogen activator, urokinase (PLAU), or any combination of the foregoing.

In some embodiments, an EWSR1::ERG target gene comprises TGFBR2.

In some embodiments, an EWSR1::ERG target gene comprises BMP4.

In some embodiments, an EWSR1::ERG target gene comprises PLAU.

In some embodiments, an EWSR1::ERG target gene comprises TGFBR2, BMP4, or PLAU.

In some embodiments, an EWSR1::ERG target gene comprises TGFBR2, BMP4, and PLAU.

In some embodiments, an agent (e.g., ITZ or an analog thereof) or a pharmaceutically acceptable salt thereof:
a) reduces expression of GLI1;
b) reduces binding of GLI1 to EWSR1;
c) reduces expression of EWSR1;
d) reduces binding of GLI1 to EWSR1::FLI1;
e) reduces expression of EWSR1::FLI1;
f) reduces expression of an EWSR1::FLI1 target gene;
g) reduces binding of GLI1 to EWSR1::ERG;
h) reduces expression of EWSR1::ERG;
i) reduces expression of an EWSR1::ERG target gene;
or any combination of the foregoing (e.g., in a subject).

In some embodiments, an agent (e.g., ITZ or an analog thereof) or a pharmaceutically acceptable salt thereof:
a) reduces expression of GLI2;
b) reduces binding of GLI2 to EWSR1;
c) reduces expression of EWSR1;
d) reduces binding of GLI2 to EWSR1::FLI1;
e) reduces expression of EWSR1::FLI1;
f) reduces expression of an EWSR1::FLI1 target gene;
g) reduces binding of GLI2 to EWSR1::ERG;
h) reduces expression of EWSR1::ERG;
i) reduces expression of an EWSR1::ERG target gene;
or any combination of the foregoing (e.g., in a subject).

In some embodiments, an agent (e.g., ITZ or an analog thereof) or a pharmaceutically acceptable salt thereof:
a) reduces expression of GLI1;
b) reduces binding of GLI1 to EWSR1;
c) reduces binding of GLI1 to EWSR1::FLI1;
d) reduces binding of GLI1 to EWSR1::ERG;
e) reduces expression of GLI2;
f) reduces binding of GLI2 to EWSR1;
g) reduces binding of GLI2 to EWSR1::FLI1;
h) reduces binding of GLI2 to EWSR1::ERG;
i) reduces expression of EWSR1;
j) reduces expression of EWSR1::FLI1;
k) reduces expression of an EWSR1::FLI1 target gene;
1) reduces expression of EWSR1::ERG;
m) reduces expression of an EWSR1::ERG target gene;
or any combination of the foregoing (e.g., in a subject).

In some embodiments, an agent (e.g., ITZ or an analog thereof) or a pharmaceutically acceptable salt thereof reduces expression of EWSR1::FLI1, EWSR1::ERG, or both (e.g., in a subject), and:
a) reduces expression of H3 clustered histone 10 (H3C10), zinc finger and BTB domain containing 21 (ZBTB21), histone cell cycle regulator (HIRA), activating transcription factor 6 beta (ATF6B), linker for activation of T cells (LAT), dedicator of cytokinesis 11 (DOCK11), fucosyltransferase 3 (FUT3), 3-hydroxy-3-methylglutaryl-CoA lyase (HMGCL), C1GALT1, C1GALT1-specific chaperone 1 (C1GALT1C1), SMO, GLI1, GLI2, or any combination of the foregoing (e.g., in a subject);
b) reduces activity of H3C10, ZBTB21, HIRA, ATF6B, LAT, DOCK11, FUT3, HMGCL, C1GALT1, C1GALT1C1, SMO, GLI1, GLI2, or any combination of the foregoing (e.g., in a subject);
or both a) and b).

In some embodiments, an agent (e.g., ITZ or an analog thereof) or a pharmaceutically acceptable salt thereof reduces expression of EWSR1::FLI1, EWSR1::ERG, or both, and reduces expression of H3C10, ZBTB21, HIRA, ATF6B, LAT, DOCK11, FUT3, HMGCL, C1GALT1, C1GALT1C1, SMO, GLI1, GLI2, or any combination of the foregoing (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression of H3C10 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression of ZBTB21 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression of HIRA (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression of ATF6B (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression of LAT (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression of DOCK11 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression of FUT3 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression of HMGCL (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression of C1GALT1 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression of C1GALT1C1 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression of SMO (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression of GLI1 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression of GLI2 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression of H3C10, ZBTB21, HIRA, ATF6B, LAT, DOCK11, FUT3, HMGCL, C1GALT1, C1GALT1C1, SMO, GLI1, and GLI2 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces activity of H3C10, ZBTB21, HIRA, ATF6B, LAT, DOCK11, FUT3, HMGCL, C1GALT1, C1GALT1C1, SMO, GLI1, GLI2, or any combination of the foregoing (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces activity of H3C10 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces activity of ZBTB21 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces activity of HIRA (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces activity of ATF6B (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces activity of LAT (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces activity of DOCK11 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces activity of FUT3 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces activity of HMGCL (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces activity of C1GALT1 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces activity of C1GALT1C1 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces activity of SMO (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces activity of GLI1 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces activity of GLI2 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces activity of H3C10, ZBTB21, HIRA, ATF6B, LAT, DOCK11, FUT3, HMGCL, C1GALT1, C1GALT1C1, SMO, GLI1, and GLI2 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces activity of H3C10, ZBTB21, HIRA, ATF6B, LAT, DOCK11, FUT3, HMGCL, C1GALT1, C1GALT1C1, SMO, GLI1, or GLI2 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression and activity of H3C10, ZBTB21, HIRA, ATF6B, LAT, DOCK11, FUT3, HMGCL, C1GALT1, C1GALT1C1, SMO, GLI1, GLI2, or any combination of the foregoing (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression and activity of H3C10 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression and activity of ZBTB21 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression and activity of HIRA (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression and activity of ATF6B (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression and activity of LAT (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression and activity of DOCK11 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression and activity of FUT3 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression and activity of HMGCL (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression and activity of C1GALT1 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression and activity of C1GALT1C1 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression and activity of SMO (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression and activity of GLI1 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression and activity of GLI2 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression and activity of H3C10, ZBTB21, HIRA, ATF6B, LAT, DOCK11, FUT3, HMGCL, C1GALT1, C1GALT1C1, SMO, GLI1, and GLI2 (e.g., in a subject).

In some embodiments, an agent (e.g., ITZ or an analog thereof) or a pharmaceutically acceptable salt thereof reduces expression of EWSR1::FLI1, EWSR1::ERG, or both, and reduces expression and activity of H3C10, ZBTB21, HIRA, ATF6B, LAT, DOCK11, FUT3, HMGCL, C1GALT1, C1GALT1C1, SMO, GLI1, or GLI2 (e.g., in a subject).

In some embodiments, an agent (e.g., ITZ or an analog thereof) or a pharmaceutically acceptable salt thereof reduces expression of EWSR1::FLI1 and reduces expression of H3C10, ZBTB21, HIRA, ATF6B, LAT, DOCK11, FUT3, HMGCL, C1GALT1, C1GALT1C1, SMO, GLI1, GLI2, or any combination of the foregoing (e.g., in a subject).

In some embodiments, an agent (e.g., ITZ or an analog thereof) or a pharmaceutically acceptable salt thereof reduces expression of EWSR1::FLI1 and reduces expression of H3C10 (e.g., in a subject).

In some embodiments, an agent (e.g., ITZ or an analog thereof) or a pharmaceutically acceptable salt thereof reduces expression of EWSR1::FLI1 and reduces expression of ZBTB21 (e.g., in a subject).

In some embodiments, an agent (e.g., ITZ or an analog thereof) or a pharmaceutically acceptable salt thereof reduces expression of EWSR1::FLI1 and reduces expression of HIRA (e.g., in a subject).

In some embodiments, an agent (e.g., ITZ or an analog thereof) or a pharmaceutically acceptable salt thereof reduces expression of EWSR1::FLI1 and reduces expression of ATF6B (e.g., in a subject).

In some embodiments, an agent (e.g., ITZ or an analog thereof) or a pharmaceutically acceptable salt thereof reduces expression of EWSR1::FLI1 and reduces expression of LAT (e.g., in a subject).

In some embodiments, an agent (e.g., ITZ or an analog thereof) or a pharmaceutically acceptable salt thereof reduces expression of EWSR1::FLI1 and reduces expression of DOCK11 (e.g., in a subject).

In some embodiments, an agent (e.g., ITZ or an analog thereof) or a pharmaceutically acceptable salt thereof reduces expression of EWSR1::FLI1 and reduces expression of FUT3 (e.g., in a subject).

In some embodiments, an agent (e.g., ITZ or an analog thereof) or a pharmaceutically acceptable salt thereof reduces expression of EWSR1::FLI1 and reduces expression of HMGCL (e.g., in a subject).

In some embodiments, an agent (e.g., ITZ or an analog thereof) or a pharmaceutically acceptable salt thereof reduces expression of EWSR1::FLI1 and reduces expression of C1GALT1 (e.g., in a subject).

In some embodiments, an agent (e.g., ITZ or an analog thereof) or a pharmaceutically acceptable salt thereof reduces expression of EWSR1::FLI1 and reduces expression of C1GALT1C1 (e.g., in a subject).

In some embodiments, an agent (e.g., ITZ or an analog thereof) or a pharmaceutically acceptable salt thereof reduces expression of EWSR1::FLI1 and reduces expression of SMO (e.g., in a subject).

In some embodiments, an agent (e.g., ITZ or an analog thereof) or a pharmaceutically acceptable salt thereof reduces expression of EWSR1::FLI1 and reduces expression of GLI1 (e.g., in a subject).

In some embodiments, an agent (e.g., ITZ or an analog thereof) or a pharmaceutically acceptable salt thereof reduces expression of EWSR1::FLI1 and reduces expression of GLI2 (e.g., in a subject).

In some embodiments, an agent (e.g., ITZ or an analog thereof) or a pharmaceutically acceptable salt thereof reduces expression of EWSR1::FLI1 and reduces expression of H3C10, ZBTB21, HIRA, ATF6B, LAT, DOCK11, FUT3, HMGCL, C1GALT1, C1GALT1C1, SMO, GLI1, and GLI2 (e.g., in a subject).

In some embodiments, an agent (e.g., ITZ or an analog thereof) or a pharmaceutically acceptable salt thereof reduces expression of EWSR1::FLI1 and reduces activity of H3C10, ZBTB21, HIRA, ATF6B, LAT, DOCK11, FUT3, HMGCL, C1GALT1, C1GALT1C1, SMO, GLI1, GLI2, or any combination of the foregoing (e.g., in a subject).

In some embodiments, an agent (e.g., ITZ or an analog thereof) or a pharmaceutically acceptable salt thereof reduces expression of EWSR1::FLI1 and reduces activity of H3C10 (e.g., in a subject).

In some embodiments, an agent (e.g., ITZ or an analog thereof) or a pharmaceutically acceptable salt thereof reduces expression of EWSR1::FLI1 and reduces activity of ZBTB21 (e.g., in a subject).

In some embodiments, an agent (e.g., ITZ or an analog thereof) or a pharmaceutically acceptable salt thereof reduces expression of EWSR1::FLI1 and reduces activity of HIRA (e.g., in a subject).

In some embodiments, an agent (e.g., ITZ or an analog thereof) or a pharmaceutically acceptable salt thereof reduces expression of EWSR1::FLI1 and reduces activity of ATF6B (e.g., in a subject).

In some embodiments, an agent (e.g., ITZ or an analog thereof) or a pharmaceutically acceptable salt thereof reduces expression of EWSR1::FLI1 and reduces activity of LAT (e.g., in a subject).

In some embodiments, an agent (e.g., ITZ or an analog thereof) or a pharmaceutically acceptable salt thereof reduces expression of EWSR1::FLI1 and reduces activity of DOCK11 (e.g., in a subject).

In some embodiments, an agent (e.g., ITZ or an analog thereof) or a pharmaceutically acceptable salt thereof reduces expression of EWSR1::FLI1 and reduces activity of FUT3 (e.g., in a subject).

In some embodiments, an agent (e.g., ITZ or an analog thereof) or a pharmaceutically acceptable salt thereof reduces expression of EWSR1::FLI1 and reduces activity of HMGCL (e.g., in a subject).

In some embodiments, an agent (e.g., ITZ or an analog thereof) or a pharmaceutically acceptable salt thereof reduces expression of EWSR1::FLI1 and reduces activity of C1GALT1 (e.g., in a subject).

In some embodiments, an agent (e.g., ITZ or an analog thereof) or a pharmaceutically acceptable salt thereof reduces expression of EWSR1::FLI1 and reduces activity of C1GALT1C1 (e.g., in a subject).

In some embodiments, an agent (e.g., ITZ or an analog thereof) or a pharmaceutically acceptable salt thereof reduces expression of EWSR1::FLI1 and reduces activity of SMO (e.g., in a subject).

In some embodiments, an agent (e.g., ITZ or an analog thereof) or a pharmaceutically acceptable salt thereof reduces expression of EWSR1::FLI1 and reduces activity of GLI1 (e.g., in a subject).

In some embodiments, an agent (e.g., ITZ or an analog thereof) or a pharmaceutically acceptable salt thereof reduces expression of EWSR1::FLI1 and reduces activity of GLI2 (e.g., in a subject).

In some embodiments, an agent (e.g., ITZ or an analog thereof) or a pharmaceutically acceptable salt thereof reduces expression of EWSR1::FLI1 and reduces activity of H3C10, ZBTB21, HIRA, ATF6B, LAT, DOCK11, FUT3, HMGCL, C1GALT1, C1GALT1C1, SMO, GLI1, and GLI2 (e.g., in a subject).

In some embodiments, an agent (e.g., ITZ or an analog thereof) or a pharmaceutically acceptable salt thereof reduces expression of EWSR1::FLI1 and reduces activity of H3C10, ZBTB21, HIRA, ATF6B, LAT, DOCK11, FUT3, HMGCL, C1GALT1, C1GALTIC1, SMO, GLI1, or GLI2 (e.g., in a subject).

In some embodiments, an agent (e.g., ITZ or an analog thereof) or a pharmaceutically acceptable salt thereof reduces expression of EWSR1::FLI1 and reduces expression and activity of H3C10, ZBTB21, HIRA, ATF6B, LAT, DOCK11, FUT3, HMGCL, C1GALT1, C1GALT1C1, SMO, GLI1, GLI2, or any combination of the foregoing (e.g., in a subject).

In some embodiments, an agent (e.g., ITZ or an analog thereof) or a pharmaceutically acceptable salt thereof reduces expression of EWSR1::FLI1 and reduces expression and activity of H3C10 (e.g., in a subject).

In some embodiments, an agent (e.g., ITZ or an analog thereof) or a pharmaceutically acceptable salt thereof reduces expression of EWSR1::FLI1 and reduces expression and activity of ZBTB21 (e.g., in a subject).

In some embodiments, an agent (e.g., ITZ or an analog thereof) or a pharmaceutically acceptable salt thereof reduces expression of EWSR1::FLI1 and reduces expression and activity of HIRA (e.g., in a subject).

In some embodiments, an agent (e.g., ITZ or an analog thereof) or a pharmaceutically acceptable salt thereof reduces expression of EWSR1::FLI1 and reduces expression and activity of ATF6B (e.g., in a subject).

In some embodiments, an agent (e.g., ITZ or an analog thereof) or a pharmaceutically acceptable salt thereof reduces expression of EWSR1::FLI1 and reduces expression and activity of LAT (e.g., in a subject).

In some embodiments, an agent (e.g., ITZ or an analog thereof) or a pharmaceutically acceptable salt thereof reduces expression of EWSR1::FLI1 and reduces expression and activity of DOCK11 (e.g., in a subject).

In some embodiments, an agent (e.g., ITZ or an analog thereof) or a pharmaceutically acceptable salt thereof reduces expression of EWSR1::FLI1 and reduces expression and activity of FUT3 (e.g., in a subject).

In some embodiments, an agent (e.g., ITZ or an analog thereof) or a pharmaceutically acceptable salt thereof reduces expression of EWSR1::FLI1 and reduces expression and activity of HMGCL (e.g., in a subject).

In some embodiments, an agent (e.g., ITZ or an analog thereof) or a pharmaceutically acceptable salt thereof reduces expression of EWSR1::FLI1 and reduces expression and activity of C1GALT1 (e.g., in a subject).

In some embodiments, an agent (e.g., ITZ or an analog thereof) or a pharmaceutically acceptable salt thereof reduces expression of EWSR1::FLI1 and reduces expression and activity of C1GALT1C1 (e.g., in a subject).

In some embodiments, an agent (e.g., ITZ or an analog thereof) or a pharmaceutically acceptable salt thereof reduces expression of EWSR1::FLI1 and reduces expression and activity of SMO (e.g., in a subject).

In some embodiments, an agent (e.g., ITZ or an analog thereof) or a pharmaceutically acceptable salt thereof reduces expression of EWSR1::FLI1 and reduces expression and activity of GLI1 (e.g., in a subject).

In some embodiments, an agent (e.g., ITZ or an analog thereof) or a pharmaceutically acceptable salt thereof reduces expression of EWSR1::FLI1 and reduces expression and activity of GLI2 (e.g., in a subject).

In some embodiments, an agent (e.g., ITZ or an analog thereof) or a pharmaceutically acceptable salt thereof reduces expression of EWSR1::FLI1 and reduces expression and activity of H3C10, ZBTB21, HIRA, ATF6B, LAT, DOCK11, FUT3, HMGCL, C1GALT1, C1GALT1C1, SMO, GLI1, and GLI2 (e.g., in a subject).

In some embodiments, an agent (e.g., ITZ or an analog thereof) or a pharmaceutically acceptable salt thereof reduces expression of EWSR1::FLI1 and reduces expression and activity of H3C10, ZBTB21, HIRA, ATF6B, LAT, DOCK11, FUT3, HMGCL, C1GALT1, C1GALT1C1, SMO, GLI1, or GLI2 (e.g., in a subject).

In some embodiments, an agent (e.g., ITZ or an analog thereof) or a pharmaceutically acceptable salt thereof reduces expression of EWSR1::ERG and reduces expression of H3C10, ZBTB21, HIRA, ATF6B, LAT, DOCK11, FUT3, HMGCL, C1GALT1, C1GALT1C1, SMO, GLI1, GLI2, or any combination of the foregoing (e.g., in a subject).

In some embodiments, an agent (e.g., ITZ or an analog thereof) or a pharmaceutically acceptable salt thereof reduces expression of EWSR1::ERG and reduces expression of H3C10 (e.g., in a subject).

In some embodiments, an agent (e.g., ITZ or an analog thereof) or a pharmaceutically acceptable salt thereof reduces expression of EWSR1::ERG and reduces expression of ZBTB21 (e.g., in a subject).

In some embodiments, an agent (e.g., ITZ or an analog thereof) or a pharmaceutically acceptable salt thereof reduces expression of EWSR1::ERG and reduces expression of HIRA (e.g., in a subject).

In some embodiments, an agent (e.g., ITZ or an analog thereof) or a pharmaceutically acceptable salt thereof reduces expression of EWSR1::ERG and reduces expression of ATF6B (e.g., in a subject).

In some embodiments, an agent (e.g., ITZ or an analog thereof) or a pharmaceutically acceptable salt thereof reduces expression of EWSR1::ERG and reduces expression of LAT (e.g., in a subject).

In some embodiments, an agent (e.g., ITZ or an analog thereof) or a pharmaceutically acceptable salt thereof reduces expression of EWSR1::ERG and reduces expression of DOCK11 (e.g., in a subject).

In some embodiments, an agent (e.g., ITZ or an analog thereof) or a pharmaceutically acceptable salt thereof reduces expression of EWSR1::ERG and reduces expression of FUT3 (e.g., in a subject).

In some embodiments, an agent (e.g., ITZ or an analog thereof) or a pharmaceutically acceptable salt thereof reduces expression of EWSR1::ERG and reduces expression of HMGCL (e.g., in a subject).

In some embodiments, an agent (e.g., ITZ or an analog thereof) or a pharmaceutically acceptable salt thereof reduces expression of EWSR1::ERG and reduces expression of C1GALT1 (e.g., in a subject).

In some embodiments, an agent (e.g., ITZ or an analog thereof) or a pharmaceutically acceptable salt thereof reduces expression of EWSR1::ERG and reduces expression of C1GALT1C1 (e.g., in a subject).

In some embodiments, an agent (e.g., ITZ or an analog thereof) or a pharmaceutically acceptable salt thereof reduces expression of EWSR1::ERG and reduces expression of SMO (e.g., in a subject).

In some embodiments, an agent (e.g., ITZ or an analog thereof) or a pharmaceutically acceptable salt thereof reduces expression of EWSR1::ERG and reduces expression of GLI1 (e.g., in a subject).

In some embodiments, an agent (e.g., ITZ or an analog thereof) or a pharmaceutically acceptable salt thereof reduces expression of EWSR1::ERG and reduces expression of GLI2 (e.g., in a subject).

In some embodiments, an agent (e.g., ITZ or an analog thereof) or a pharmaceutically acceptable salt thereof reduces expression of EWSR1::ERG and reduces expression of H3C10, ZBTB21, HIRA, ATF6B, LAT, DOCK11, FUT3, HMGCL, C1GALT1, C1GALT1C1, SMO, GLI1, and GLI2 (e.g., in a subject).

In some embodiments, an agent (e.g., ITZ or an analog thereof) or a pharmaceutically acceptable salt thereof reduces expression of EWSR1::ERG and reduces expression of H3C10, ZBTB21, HIRA, ATF6B, LAT, DOCK11, FUT3, HMGCL, C1GALT1, C1GALT1C1, SMO, GLI1, or GLI2 (e.g., in a subject).

In some embodiments, an agent (e.g., ITZ or an analog thereof) or a pharmaceutically acceptable salt thereof reduces expression of EWSR1::ERG and reduces activity of H3C10, ZBTB21, HIRA, ATF6B, LAT, DOCK11, FUT3, HMGCL, C1GALT1, C1GALT1C1, SMO, GLI1, GLI2, or any combination of the foregoing (e.g., in a subject).

In some embodiments, an agent (e.g., ITZ or an analog thereof) or a pharmaceutically acceptable salt thereof reduces expression of EWSR1::ERG and reduces activity of H3C10 (e.g., in a subject).

In some embodiments, an agent (e.g., ITZ or an analog thereof) or a pharmaceutically acceptable salt thereof reduces expression of EWSR1::ERG and reduces activity of ZBTB21 (e.g., in a subject).

In some embodiments, an agent (e.g., ITZ or an analog thereof) or a pharmaceutically acceptable salt thereof reduces expression of EWSR1::ERG and reduces activity of HIRA (e.g., in a subject).

In some embodiments, an agent (e.g., ITZ or an analog thereof) or a pharmaceutically acceptable salt thereof reduces expression of EWSR1::ERG and reduces activity of ATF6B (e.g., in a subject).

In some embodiments, an agent (e.g., ITZ or an analog thereof) or a pharmaceutically acceptable salt thereof reduces expression of EWSR1::ERG and reduces activity of LAT (e.g., in a subject).

In some embodiments, an agent (e.g., ITZ or an analog thereof) or a pharmaceutically acceptable salt thereof reduces expression of EWSR1::ERG and reduces activity of DOCK11 (e.g., in a subject).

In some embodiments, an agent (e.g., ITZ or an analog thereof) or a pharmaceutically acceptable salt thereof reduces expression of EWSR1::ERG and reduces activity of FUT3 (e.g., in a subject).

In some embodiments, an agent (e.g., ITZ or an analog thereof) or a pharmaceutically acceptable salt thereof reduces expression of EWSR1::ERG and reduces activity of HMGCL (e.g., in a subject).

In some embodiments, an agent (e.g., ITZ or an analog thereof) or a pharmaceutically acceptable salt thereof reduces expression of EWSR1::ERG and reduces activity of C1GALT1 (e.g., in a subject).

In some embodiments, an agent (e.g., ITZ or an analog thereof) or a pharmaceutically acceptable salt thereof reduces expression of EWSR1::ERG and reduces activity of C1GALT1C1 (e.g., in a subject).

In some embodiments, an agent (e.g., ITZ or an analog thereof) or a pharmaceutically acceptable salt thereof reduces expression of EWSR1::ERG and reduces activity of SMO (e.g., in a subject).

In some embodiments, an agent (e.g., ITZ or an analog thereof) or a pharmaceutically acceptable salt thereof reduces expression of EWSR1::ERG and reduces activity of GLI1 (e.g., in a subject).

In some embodiments, an agent (e.g., ITZ or an analog thereof) or a pharmaceutically acceptable salt thereof reduces expression of EWSR1::ERG and reduces activity of GLI2 (e.g., in a subject).

In some embodiments, an agent (e.g., ITZ or an analog thereof) or a pharmaceutically acceptable salt thereof reduces expression of EWSR1::ERG and reduces activity of H3C10, ZBTB21, HIRA, ATF6B, LAT, DOCK11, FUT3, HMGCL, C1GALT1, C1GALT1C1, SMO, GLI1, and GLI2 (e.g., in a subject).

In some embodiments, an agent (e.g., ITZ or an analog thereof) or a pharmaceutically acceptable salt thereof reduces expression of EWSR1::ERG and reduces activity of H3C10, ZBTB21, HIRA, ATF6B, LAT, DOCK11, FUT3, HMGCL, C1GALT1, C1GALT1C1, SMO, GLI1, or GLI2 (e.g., in a subject).

In some embodiments, an agent (e.g., ITZ or an analog thereof) or a pharmaceutically acceptable salt thereof reduces expression of EWSR1::ERG and reduces expression and activity of H3C10, ZBTB21, HIRA, ATF6B, LAT, DOCK11, FUT3, HMGCL, C1GALT1, C1GALT1C1, SMO, GLI1, GLI2, or any combination of the foregoing (e.g., in a subject).

In some embodiments, an agent (e.g., ITZ or an analog thereof) or a pharmaceutically acceptable salt thereof reduces expression of EWSR1::ERG and reduces expression and activity of H3C10 (e.g., in a subject).

In some embodiments, an agent (e.g., ITZ or an analog thereof) or a pharmaceutically acceptable salt thereof reduces expression of EWSR1::ERG and reduces expression and activity of ZBTB21 (e.g., in a subject).

In some embodiments, an agent (e.g., ITZ or an analog thereof) or a pharmaceutically acceptable salt thereof reduces expression of EWSR1::ERG and reduces expression and activity of HIRA (e.g., in a subject).

In some embodiments, an agent (e.g., ITZ or an analog thereof) or a pharmaceutically acceptable salt thereof reduces expression of EWSR1::ERG and reduces expression and activity of ATF6B (e.g., in a subject).

In some embodiments, an agent (e.g., ITZ or an analog thereof) or a pharmaceutically acceptable salt thereof reduces expression of EWSR1::ERG and reduces expression and activity of LAT (e.g., in a subject).

In some embodiments, an agent (e.g., ITZ or an analog thereof) or a pharmaceutically acceptable salt thereof reduces expression of EWSR1::ERG and reduces expression and activity of DOCK11 (e.g., in a subject).

In some embodiments, an agent (e.g., ITZ or an analog thereof) or a pharmaceutically acceptable salt thereof reduces expression of EWSR1::ERG and reduces expression and activity of FUT3 (e.g., in a subject).

In some embodiments, an agent (e.g., ITZ or an analog thereof) or a pharmaceutically acceptable salt thereof reduces expression of EWSR1::ERG and reduces expression and activity of HMGCL (e.g., in a subject).

In some embodiments, an agent (e.g., ITZ or an analog thereof) or a pharmaceutically acceptable salt thereof reduces expression of EWSR1::ERG and reduces expression and activity of C1GALT1 (e.g., in a subject).

In some embodiments, an agent (e.g., ITZ or an analog thereof) or a pharmaceutically acceptable salt thereof reduces expression of EWSR1::ERG and reduces expression and activity of C1GALT1C1 (e.g., in a subject).

In some embodiments, an agent (e.g., ITZ or an analog thereof) or a pharmaceutically acceptable salt thereof reduces expression of EWSR1::ERG and reduces expression and activity of SMO (e.g., in a subject).

In some embodiments, an agent (e.g., ITZ or an analog thereof) or a pharmaceutically acceptable salt thereof reduces expression of EWSR1::ERG and reduces expression and activity of GLI1 (e.g., in a subject).

In some embodiments, an agent (e.g., ITZ or an analog thereof) or a pharmaceutically acceptable salt thereof reduces expression of EWSR1::ERG and reduces expression and activity of GLI2 (e.g., in a subject).

In some embodiments, an agent (e.g., ITZ or an analog thereof) or a pharmaceutically acceptable salt thereof reduces expression of EWSR1::ERG and reduces expression and activity of H3C10, ZBTB21, HIRA, ATF6B, LAT, DOCK11, FUT3, HMGCL, C1GALT1, C1GALT1C1, SMO, GLI1, and GLI2 (e.g., in a subject).

In some embodiments, an agent (e.g., ITZ or an analog thereof) or a pharmaceutically acceptable salt thereof reduces expression of EWSR1::ERG and reduces expression and activity of H3C10, ZBTB21, HIRA, ATF6B, LAT, DOCK11, FUT3, HMGCL, C1GALT1, C1GALT1C1, SMO, GLI1, or GLI2 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression of H3C10, ZBTB21, HIRA, ATF6B, LAT, DOCK11, FUT3, HMGCL, C1GALT1, C1GALT1C1, SMO, GLI1, GLI2, or any combination of the foregoing (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression of H3C10 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression of ZBTB21 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression of HIRA (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression of ATF6B (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression of LAT (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression of DOCK11 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression of FUT3 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression of HMGCL (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression of C1GALT1 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression of C1GALT1C1 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression of SMO (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression of GLI1 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression of GLI2 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression of H3C10, ZBTB21, HIRA, ATF6B, LAT, DOCK11, FUT3, HMGCL, C1GALT1, C1GALT1C1, SMO, GLI1, and GLI2 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression of H3C10, ZBTB21, HIRA, ATF6B, LAT, DOCK11, FUT3, HMGCL, C1GALT1, C1GALT1C1, SMO, GLI1, or GLI2 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces activity of H3C10, ZBTB21, HIRA, ATF6B, LAT, DOCK11, FUT3, HMGCL, C1GALT1, C1GALT1C1, SMO, GLI1, GLI2, or any combination of the foregoing (e.g., in a subject).

In some embodiments, an agent (e.g., ITZ or an analog thereof) or a pharmaceutically acceptable salt thereof reduces expression of both EWSR1::FLI1 and EWSR1::ERG, and reduces activity of H3C10 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces activity of ZBTB21 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces activity of HIRA (e.g., in a subject).

In some embodiments, an agent (e.g., ITZ or an analog thereof) or a pharmaceutically acceptable salt thereof reduces expression of both EWSR1::FLI1 and EWSR1::ERG, and reduces activity of ATF6B (e.g., in a subject).

In some embodiments, an agent (e.g., ITZ or an analog thereof) or a pharmaceutically acceptable salt thereof reduces expression of both EWSR1::FLI1 and EWSR1::ERG, and reduces activity of LAT (e.g., in a subject).

In some embodiments, an agent (e.g., ITZ or an analog thereof) or a pharmaceutically acceptable salt thereof reduces expression of both EWSR1::FLI1 and EWSR1::ERG, and reduces activity of DOCK11 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces activity of FUT3 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces activity of HMGCL (e.g., in a subject).

In some embodiments, an agent (e.g., ITZ or an analog thereof) or a pharmaceutically acceptable salt thereof reduces expression of both EWSR1::FLI1 and EWSR1::ERG, and reduces activity of C1GALT1 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces activity of C1GALT1C1 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces activity of SMO (e.g., in a subject).

In some embodiments, an agent (e.g., ITZ or an analog thereof) or a pharmaceutically acceptable salt thereof reduces expression of both EWSR1::FLI1 and EWSR1::ERG, and reduces activity of GLI1 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces activity of GLI2 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces activity of H3C10, ZBTB21, HIRA, ATF6B, LAT, DOCK11, FUT3, HMGCL, C1GALT1, C1GALT1C1, SMO, GLI1, and GLI2 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces activity of H3C10, ZBTB21, HIRA, ATF6B, LAT, DOCK11, FUT3, HMGCL, C1GALT1, C1GALT1C1, SMO, GLI1, or GLI2 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression and activity of H3C10, ZBTB21, HIRA, ATF6B, LAT, DOCK11, FUT3, HMGCL, C1GALT1, C1GALT1C1, SMO, GLI1, GLI2, or any combination of the foregoing (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression and activity of H3C10 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression and activity of ZBTB21 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression and activity of HIRA (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression and activity of ATF6B (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression and activity of LAT (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression and activity of DOCK11 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression and activity of FUT3 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression and activity of HMGCL (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression and activity of C1GALT1 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression and activity of C1GALT1C1 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression and activity of SMO (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression and activity of GLI1 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression and activity of GLI2 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression and activity of H3C10, ZBTB21, HIRA, ATF6B, LAT, DOCK11, FUT3, HMGCL, C1GALT1, C1GALT1C1, SMO, GLI1, and GLI2 (e.g., in a subject).

In some embodiments, an agent (e.g., ITZ or an analog thereof) or a pharmaceutically acceptable salt thereof reduces expression of both EWSR1::FLI1 and EWSR1::ERG, and reduces expression and activity of H3C10, ZBTB21, HIRA, ATF6B, LAT, DOCK11, FUT3, HMGCL, C1GALT1, C1GALT1C1, SMO, GLI1, or GLI2 (e.g., in a subject).

In some embodiments, an EWSR1::FLI1 target gene comprises G protein-coupled receptor kinase 5 (GRK5), nuclear receptor subfamily 0 group b member 1 (NR0B1), vaccinia-related kinase 1 (VRK1), cyclin-dependent kinase inhibitor 1A (CDKN1A), fos-related antigen 2 (FOSL2), forkhead box protein 01 (FOXO1), or any combination of the foregoing.

In some embodiments, an EWSR1::FLI1 target gene comprises GRK5.

In some embodiments, an EWSR1::FLI1 target gene comprises NROB1.

In some embodiments, an EWSR1::FLI1 target gene comprises VRK1.

In some embodiments, an EWSR1::FLI1 target gene comprises CDKN1A.

In some embodiments, an EWSR1::FLI1 target gene comprises FOSL2.

In some embodiments, an EWSR1::FLI1 target gene comprises FOXO1.

In some embodiments, an EWSR1::FLI1 target gene comprises GRK5, NR0B1, VRK1, CDKN1A, FOSL2, or FOXO1.

In some embodiments, an EWSR1::FLI1 target gene comprises GRK5, NR0B1, VRK1, CDKN1A, FOSL2, and FOXO1.

In some embodiments, an agent (e.g., ITZ or an analog thereof) or a pharmaceutically acceptable salt thereof:
a) reduces expression of C1GALT1,
b) reduces O-galactosylation of Smoothened (SMO),
c) reduces expression of SMO,
d) reduces ligand-independent hedgehog signaling,
e) reduces expression of GLI1,
f) reduces binding of GLI1 to EWS-FLI1,
g) reduces expression of EWS-FLI1, or
h) reduces expression of an EWS-FLI1 target gene,
or any combination of the foregoing.

In some embodiments, an agent (e.g., ITZ or an analog thereof) or a pharmaceutically acceptable salt thereof reduces expression of C1GALT1.

In some embodiments, an agent (e.g., ITZ or an analog thereof) or a pharmaceutically acceptable salt thereof reduces O-galactosylation of SMO.

In some embodiments, an agent (e.g., ITZ or an analog thereof) or a pharmaceutically acceptable salt thereof reduces expression of SMO.

In some embodiments, an agent (e.g., ITZ or an analog thereof) or a pharmaceutically acceptable salt thereof:
a) reduces O-galactosylation of Smoothened (SMO),
b) reduces expression of SMO,
or both of the foregoing.

In some embodiments, an agent (e.g., ITZ or an analog thereof) or a pharmaceutically acceptable salt thereof reduces ligand-independent hedgehog signaling.

In some embodiments, an agent (e.g., ITZ or an analog thereof) or a pharmaceutically acceptable salt thereof reduces expression of GLI1.

In some embodiments, an agent (e.g., ITZ or an analog thereof) or a pharmaceutically acceptable salt thereof reduces binding of GLI1 to EWS-FLI1.

In some embodiments, an agent (e.g., ITZ or an analog thereof) or a pharmaceutically acceptable salt thereof:
a) reduces expression of GLI1,
b) reduces binding of GLI1 to EWS-FLI1,
or both of the foregoing.

In some embodiments, an agent (e.g., ITZ or an analog thereof) or a pharmaceutically acceptable salt thereof reduces expression of EWS-FLI1.

In some embodiments, an agent (e.g., ITZ or an analog thereof) or a pharmaceutically acceptable salt thereof reduces expression of an EWS-FLI1 target gene.

In some embodiments, an agent (e.g., ITZ or an analog thereof) or a pharmaceutically acceptable salt thereof:
a) reduces expression of EWS-FLI1, or
b) reduces expression of an EWS-FLI1 target gene,
or both of the foregoing.

In some embodiments, an agent (e.g., ITZ or an analog thereof) or a pharmaceutically acceptable salt thereof:
a) reduces expression of GLI1,
b) reduces binding of GLI1 to EWS-FLI1,
c) reduces expression of EWS-FLI1, or
d) reduces expression of an EWS-FLI1 target gene,
or any combination of the foregoing.

In some embodiments, an agent (e.g., ITZ or an analog thereof) or a pharmaceutically acceptable salt thereof:
a) reduces expression of C1GALT1,
b) reduces O—N-acetylgalactosamine-glycosylation (O-GalNAc-glycosylation) of Smoothened (SMO),
c) reduces expression of SMO,
d) reduces ligand-independent hedgehog signaling,
e) reduces expression of GLI1,
f) reduces binding of GLI1 to EWSR1::FLI1,
g) reduces expression of EWSR1::FLI1, or
h) reduces expression of an EWSR1::FLI1 target gene,
or any combination of the foregoing.

In some embodiments, an agent (e.g., ITZ or an analog thereof) or a pharmaceutically acceptable salt thereof reduces expression of C1GALT1.

In some embodiments, an agent (e.g., ITZ or an analog thereof) or a pharmaceutically acceptable salt thereof reduces O-GalNAc-glycosylation of SMO.

In some embodiments, an agent (e.g., ITZ or an analog thereof) or a pharmaceutically acceptable salt thereof reduces expression of SMO.

In some embodiments, an agent (e.g., ITZ or an analog thereof) or a pharmaceutically acceptable salt thereof:
a) reduces O-GalNAc-glycosylation of SMO,
b) reduces expression of SMO,
or both of the foregoing.

In some embodiments, an agent (e.g., ITZ or an analog thereof) or a pharmaceutically acceptable salt thereof reduces ligand-independent hedgehog signaling.

In some embodiments, an agent (e.g., ITZ or an analog thereof) or a pharmaceutically acceptable salt thereof reduces expression of GLI1.

In some embodiments, an agent (e.g., ITZ or an analog thereof) or a pharmaceutically acceptable salt thereof reduces binding of GLI1 to EWSR1::FLI1.

In some embodiments, an agent (e.g., ITZ or an analog thereof) or a pharmaceutically acceptable salt thereof:
a) reduces expression of GLI1,
b) reduces binding of GLI1 to EWSR1::FLI1,
or both of the foregoing.

In some embodiments, an agent (e.g., ITZ or an analog thereof) or a pharmaceutically acceptable salt thereof reduces expression of EWSR1::FLI1.

In some embodiments, an agent (e.g., ITZ or an analog thereof) or a pharmaceutically acceptable salt thereof reduces expression of an EWSR1::FLI1 target gene.

In some embodiments, an agent (e.g., ITZ or an analog thereof) or a pharmaceutically acceptable salt thereof:
a) reduces expression of EWSR1::FLI1, or
b) reduces expression of an EWSR1::FLI1 target gene,
or both of the foregoing.

In some embodiments, an agent (e.g., ITZ or an analog thereof) or a pharmaceutically acceptable salt thereof:
a) reduces expression of GLI1,
b) reduces binding of GLI1 to EWSR1::FLI1,
c) reduces expression of EWSR1::FLI1, or
d) reduces expression of an EWSR1::FLI1 target gene,
or any combination of the foregoing.

In some embodiments, an agent comprises, consists essentially of, or consists of an itraconazole (ITZ), a derivative thereof, or an analog (analogue) thereof. In some embodiments, an agent comprises an itraconazole, a derivative thereof, or an analog thereof. In some embodiments, an agent consists essentially of an itraconazole, a derivative thereof, or an analog thereof. In some embodiments, an agent consists of an itraconazole, a derivative thereof, or an analog thereof. In some embodiments, an agent is an itraconazole, a derivative thereof, or an analog thereof.

In some embodiments, an agent comprises, consists essentially of, or consists of an itraconazole or an analog thereof. In some embodiments, an agent comprises an itraconazole or an analog thereof. In some embodiments, an agent consists essentially of an itraconazole or an analog (analogue) thereof. In some embodiments, an agent consists of an itraconazole or an analog thereof. In some embodiments, an agent is an itraconazole or an analog thereof.

In some embodiments, an agent comprises, consists essentially of, or consists of an itraconazole. In some embodiments, an agent comprises an itraconazole. In some embodiments, an agent consists essentially of an itraconazole. In some embodiments, an agent consists of an itraconazole. In some embodiments, an agent is an itraconazole.

In some embodiments, an agent comprises, consists essentially of, or consists of a derivative of an itraconazole. In some embodiments, an agent comprises a derivative of an itraconazole. In some embodiments, an agent consists essentially of a derivative of an itraconazole. In some embodiments, an agent consists of a derivative of an itraconazole. In some embodiments, an agent is a derivative of an itraconazole.

In some embodiments, an agent comprises, consists essentially of, or consists of an analog of an itraconazole. In some embodiments, an agent comprises an analog of an itraconazole. In some embodiments, an agent consists essentially of an analog of an itraconazole. In some embodiments, an agent consists of an analog of an itraconazole. In some embodiments, an agent is an analog of an itraconazole.

Itraconazole analogs are well known in the art. Non-limiting examples of itraconazole analogs are disclosed in: International Patent Application Nos. PCT/US2012/054306 (International Publication No. WO 2013036866), PCT/US2019/054583 (International Publication No. WO 2020072830), and PCT/US2015/013808 (International Publication No. WO 2015116947); and Li et al., Novel Tetrazole-Containing Analogues of Itraconazole as Potent Antiangiogenic Agents with Reduced Cytochrome P450 3A4 Inhibition, J Med Chem 61:11158-68 (2018), Li et al., Design and Synthesis of Tetrazole-and Pyridine-Containing Itraconazole Analogs as Potent Angiogenesis Inhibitors, ACS Med Chem Lett 11:1111-17 (2020), Pace et al., Structure-activity relationships for itraconazole-based triazolone analogues as Hedgehog pathway Inhibitors, J Med Chem 62:3873-85 (2019), Shi et al., Itraconazole side chain analogues: structure-activity relationship studies for inhibition of endothelial cell proliferation, vascular endothelial growth factor receptor 2 (VEGFR2) glycosylation, and hedgehog signaling, J Med Chem 54:7363-74 (2011), and Wen et al., Inhibition of hedgehog signaling by stereochemically defined des-triazole itraconazole analogues, Bioorg Med Chem Lett 30:126794 (2020), the entire contents of which are incorporated herein.

In some embodiments, an agent is an itraconazole analog selected from those listed in Table 3 in Shi et al. (2011), Tables 1 and 2 in Pace et al. (2019), and Table 1 in Wen et al. (2020).

In some embodiments, an agent is an itraconazole analog selected from those listed in Table 3 in Shi et al. (2011).

In some embodiments, an itraconazole analog is an itraconazole stereoisoform. Non-limiting examples of itraconazole analogs include: 2S,4R,2'S-itraconazole, 2S,4R,2′R-itraconazole, 2R,4S,2'S-itraconazole, 2R,4S,2′R-itraconazole, 2S,4S,2'S-itraconazole, 2S,4S,2′R-itraconazole, 2R,4R,2'S-itraconazole, 2R,4R,2′R-itraconazole, cis-(2S,4R)-4-[4-[4-[4-[[2-(2,4-Dichlorophenyl)-2-(1H-1,2,4-triazol-1-ylmethyl)-1,3-dioxolan-4-y1] methoxy]phenyl]-lpiperazinyl]phenyl]-2,4-dihydro-2-methyl-3H-1,2,4-triazol-3-one, cis-(2S,4R)-4-[4-[4-[4-[[2-(2,4-Dichlorophenyl)-2-(1H-1,2,4-triazol-1-ylmethyl)-1,3-dioxolan-4-yl] methoxy]phenyl]-lpiperazinyl]phenyl]-2,4-dihydro-2-ethyl-3H-1,2,4-triazol-3-one, cis-(2S,4R)-4-[4-[4-[4-[[2-(2,4-Dichlorophenyl)-2-(1H-1,2,4-triazol-1-ylmethyl)-1,3-dioxolan-4-yl] methoxy]phenyl]-lpiperazinyl]phenyl]-2,4-dihydro-2-propyl-3H-1,2,4-triazol-3-one, cis-(2S,4R)-4-[4-[4-[4-[[2-(2,4-Dichlorophenyl)-2-(1H-1,2,4-triazol-1-ylmethyl)-1,3-dioxolan-4-yl] methoxy]phenyl]-lpiperazinyl]phenyl]-2,4-dihydro-2-isopropyl-3H-1,2,4-triazol-3-one, cis-(2S,4R)-4-[4-[4-[4-[[2-(2,4-Dichlorophenyl)-2-(1H-1,2,4-triazol-1-ylmethyl)-1,3-dioxolan-4-yl]methoxy]phenyl]-lpiperazinyl]phenyl]-2,4-dihydro-2-butyl-3H-1,2,4-triazol-3-one, cis-(2S,4R)-4-[4-[4-[4-[[2-(2,4-Dichlorophenyl)-2-(1H-1,2,4-triazol-1-ylmethyl)-1,3-dioxolan-4-yl]methoxy]phenyl]-lpiperazinyl]phenyl]-2,4-dihydro-2-isobutyl-3H-1,2,4-triazol-3-one, cis-(2S,4R)-4-[4-[4-[4-[[2-(2,4-Dichlorophenyl)-2-(1H-1,2,4-triazol-1-ylmethyl)-1,3-dioxolan-4-yl]methoxy]phenyl]-lpiperazinyl]phenyl]-2,4-dihydro-2-pentyl-3H-1,2,4-triazol-3-one, cis-(2S,4R)-4-[4-[4-[4-[[2-(2,4-Dichlorophenyl)-2-(1H-1,2,4-triazol-1-ylmethyl)-1,3-dioxolan-4-yl]methoxy]phenyl]-lpiperazinyl]phenyl]-2,4-dihydro-2-(1-methylbutyl)-3H-1,2,4-triazol-3-one, cis-(2S,4R)-4-[4-[4-[4-[[2-(2,4-Dichlorophenyl)-2-(1H-1,2,4-triazol-1-ylmethyl)-1,3-dioxolan-4-yl]methoxy]phenyl]-lpiperazinyl]phenyl]-2,4-dihydro-2-(3-methylbutyl)-3H-1,2,4-triazol-3-one, cis-(2S,4R)-4-[4-[4-[4-[[2-(2,4-Dichlorophenyl)-2-(1H-1,2,4-triazol-1-ylmethyl)-1,3-dioxolan-4-yl]methoxy]phenyl]-lpiperazinyl]phenyl]-2,4-dihydro-2-(1-ethylpropyl)-3H-1,2,4-triazol-3-one, cis-(2S,4R)-4-[4-[4-[4-[[2-(2,4-Dichlorophenyl)-2-(1H-1,2,4-triazol-1-ylmethyl)-1,3-dioxolan-4-yl] methoxy]phenyl]-lpiperazinyl]phenyl]-2,4-dihydro-2-(1-cyclohexyl)-3H-1,2,4-triazol-3-one, cis-(2S,4R)-4-[4-[4-[4-[[2-(2,4-Dichlorophenyl)-2-(1H-1,2,4-triazol-1-ylmethyl)-1,3-dioxolan-4-yl]methoxy]phenyl]-lpiperazinyl]phenyl]-2,4-dihydro-2-(1-cyclopentylmethyl)-3H-1,2,4-triazol-3-one, cis-(2S,4R)-4-[4-[4-[4-[[2-(2,4-Dichlorophenyl)-2-(1H-1,2,4-triazol-1-ylmethyl)-1,3-dioxolan-4-yl]methoxy]phenyl]-lpiperazinyl]phenyl]-2,4-dihydro-2-(n-octyl)-3H-1,2,4-triazol-3-one, cis-(2S,4R)-4-[4-[4-[4-[[2-(2,4-Dichlorophenyl)-2-(1H-1,2,4-triazol-1-ylmethyl)-1,3-dioxolan-4-yl]methoxy]phenyl]-lpiperazinyl]phenyl]-2,4-dihydro-2-(6-phenylhexyl)-3H-1,2,4-triazol-3-one, cis-(25,4R)-4-[4-[4-[4-[[2-(2,4-Dichlorophenyl)-2-(1H-1,2,4-triazol-1-ylmethyl)-1,3-dioxolan-4-yl]methoxy]phenyl]-lpiperazinyl]phenyl]-2,4-dihydro-2-(hex-3-ynyl)-3H-1,2,4-triazol-3-one, cis-(25,4R)-4-[4-[4-[4-[[2-(2,4-Dichlorophenyl)-2-(1H-1,2,4-triazol-1-ylmethyl)-1,3-dioxolan-4-yl]methoxy]phenyl]-lpiperazinyl]phenyl]-2,4-dihydro-2-(6-azidohexyl)-3H-1,2,4-triazol-3-one, cis-(2S,4R)-4-[4-[4-[4-[[2-(2,4-Dichlorophenyl)-2-(1H-1,2,4-triazol-1-ylmethyl)-1,3-dioxolan-4-yl] methoxy]phenyl]-lpiperazinyl]phenyl]-2,4-dihydro-2-(6-(4-benzoylphenoxy)-hexyl)-3H-1,2,4-triazol-3-one, cis-(2R,4S)-4-[4-[4-[4-[[2-(2,4-Dichlorophenyl)-2-(1H-1,2,4-triazol-1-ylmethyl)-1,3-dioxolan-4-yl]methoxy]phenyl]-lpiperazinyl]phenyl]-2,4-dihydro-2-(pent-4-ynyl)-3H-1,2,4-triazol-3-one, cis-(2S,4R)-4-[4-[4-[4-[[2-(2,4-Dichlorophenyl)-2-(1H-1,2,4-triazol-1-ylmethyl)-1,3-dioxolan-4-yl]methoxy]phenyl]-lpiperazinyl]phenyl]-2,4-dihydro-2-((E)-hex-3-enyl)-3H-1,2,4-triazol-3-one, cis-(2S,4R)-4-[4-[4-[4-[[2-(2,4-Dichlorophenyl)-2-(1H-1,2,4-triazol-1-ylmethyl)-1,3-dioxolan-4-yl]methoxy]phenyl]-lpiperazinyl]phenyl]-2,4-dihydro-2-((Z)-hex-3-enyl)-3H-1,2,4-triazol-3-one, cis-(2S,4R)-4-[4-[4-[4-[[2-(2,4-Dichlorophenyl)-2-(1H-1,2,4-triazol-1-ylmethyl)-1,3-dioxolan-4-yl]methoxy]phenyl]-lpiperazinyl]phenyl]-2,4-dihydro-2-(5-cyanopentyl)-3H-1,2,4-triazol-3-one, cis-(2S,4R)-4-[4-[4-[4-[[2-(2,4-Dichlorophenyl)-2-(1H-1,2,4-triazol-1-ylmethyl)-1,3-dioxolan-4-y1] methoxy]phenyl]-lpiperazinyl]phenyl]-2,4-dihydro-2-((3-ethyloxetan-3-yl)methyl)-3H-1,2,4-triazol-3-one, cis-(2S,4R)-4-[4-[4-[4-[[2-(2,4-Dichlorophenyl)-2-(1H-1,2,4-triazol-1-ylmethyl)-1,3-dioxolan-4-yl]methoxy]phenyl]-lpiperazinyl]phenyl]-2,4-dihydro-2-(2-(3-methyl-3H-diazirin-3-yl)ethyl)-3H-1,2,4-triazol-3-one, cis-(2S,4R)-4-[4-[4-[4-[[2-(2,4-Dichlorophenyl)-2-(1H-1,2,4-triazol-1-ylmethyl)-1,3-dioxolan-4-yl]methoxy]phenyl]-lpiperazinyl]phenyl]-2,4-dihydro-2-(7-(3-methyl-3H-diazirin-3-yl) heptyl)-3H-1,2,4-triazol-3-one, or any combination of the foregoing.

In some embodiments, an itraconazole analog comprises a modified side chain. In some embodiments, a modified side chain comprises one or more functional groups. Non-limiting examples of functional groups include: azido, alkynyl, nitrile, cyclohexyl, cyclopentyl, and oxetanyl functional groups.

In some embodiments, an itraconazole analog is cis-(2R,4S)-4-[4-[4-[4-[[2-(2,4-Dichlorophenyl)-2-(1H-1,2,4-triazol-1-ylmethyl)-1,3-dioxolan-4-yl]methoxy]phenyl]-lpiperazinyl]phenyl]-2,4-dihydro-2-(pent-4-ynyl)-3H-1,2,4-triazol-3-one.

In some embodiments, an itraconazole analog is cis-(2S,4R)-4-[4-[4-[4-[[2-(2,4-Dichlorophenyl)-2-(1H-1,2,4-triazol-1-ylmethyl)-1,3-dioxolan-4-yl]methoxy]phenyl]-lpiperazinyl]phenyl]-2,4-dihydro-2-(5-cyanopentyl)-3H-1,2,4-triazol-3-one.

In some embodiments, an itraconazole analog is cis-(2S,4R)-4-[4-[4-[4-[[2-(2,4-Dichlorophenyl)-2-(1H-1,2,4-triazol-1-ylmethyl)-1,3-dioxolan-4-yl]methoxy]phenyl]-lpiperazinyl]phenyl]-2,4-dihydro-2-(6-azidohexyl)-3H-1,2,4-triazol-3-one.

In some embodiments, an itraconazole analog is cis-(2S,4R)-4-[4-[4-[4-[[2-(2,4-Dichlorophenyl)-2-(1H-1,2,4-triazol-1-ylmethyl)-1,3-dioxolan-4-yl]methoxy]phenyl]-lpiperazinyl]phenyl]-2,4-dihydro-2-(1-cyclohexyl)-3H-1,2,4-triazol-3-one.

In some embodiments, an itraconazole analog is cis-(2S,4R)-4-[4-[4-[4-[[2-(2,4-Dichlorophenyl)-2-(1H-1,2,4-triazol-1-ylmethyl)-1,3-dioxolan-4-yl]methoxy]phenyl]-lpiperazinyl]phenyl]-2,4-dihydro-2-(1-cyclopentylmethyl)-3H-1,2,4-triazol-3-one.

In some embodiments, an itraconazole analog is cis-(2S,4R)-4-[4-[4-[4-[2-(2,4-Dichlorophenyl)-2-(1H-1,2,4-triazol-1-ylmethyl)-1,3-dioxolan-4-yl]methoxyphenyl]-lpiperazinyl]phenyl]-2,4-dihydro-2-((3-ethyloxetan-3-yl)methyl)-3H-1,2,4-triazol-3-one.

In some embodiments, an agent is an itraconazole analog selected from those listed in Table 1 in Pace et al. (2019).

In some embodiments, an itraconazole analog is an itraconazole stereoisoform. Non-limiting examples of itraconazole analogs include: 1-(4-(((2R,4R)-2-(2,4-dichlorophenyl)-2-methyl-1,3-dioxolan-4-yl) methoxy)phenyl)-4-(4-nitrophenyl) piperazine, 1-(4-(((2S,4R)-2-(2,4-dichlorophenyl)-2-methyl-1,3-dioxolan-4-yl) methoxy)phenyl)-4-(4-nitrophenyl) piperazine, 4-(4-(4-(((2R,4R)-2-(2,4-dichlorophenyl)-2-methyl-1,3-dioxolan-4-yl) methoxy)phenyl) piperazin-1-yl) aniline, 4-(4-(4-(((2S,4R)-2-(2,4-dichlorophenyl)-2-methyl-1,3-dioxolan-4-yl) methoxy)phenyl) piperazin-1-yl) aniline, N-(4-(4-(4-(((2R,4R)-2-(2,4-dichlorophenyl)-2-methyl-1,3-dioxolan-4-yl) methoxy)phenyl) piperazin-1-yl)phenyl)benzamide, N-(4-(4-(4-(((2R,4R)-2-(2,4-dichlorophenyl)-2-methyl-1,3-dioxolan-4-yl) methoxy)phenyl) piperazin-1-yl)phenyl)-2-naphthamide, N-(4-(4-(4-(((2R,4R)-2-(2,4-dichlorophenyl)-2-methyl-1,3-dioxolan-4-yl) methoxy)phenyl) piperazin-1-yl)phenyl) picolinamide, N-(4-(4-(4-(((2R,4R)-2-(2,4-dichlorophenyl)-2-methyl-1,3-dioxolan-4-yl) methoxy)phenyl) piperazin-1-yl)phenyl) nicotinamide, N-(4-(4-(4-(((2R,4R)-2-(2,4-dichlorophenyl)-2-methyl-1,3-dioxolan-4-yl) methoxy)phenyl) piperazin-1-yl)phenyl) isonicotinamide, N-(4-(4-(4-(((2S,4R)-2-(2,4-dichlorophenyl)-2-methyl-1,3-dioxolan-4-yl) methoxy)phenyl) piperazin-1-yl)phenyl)benzamide, N-(4-(4-(4-(((2S,4R)-2-(2,4-dichlorophenyl)-2-methyl-1,3-dioxolan-4-yl) methoxy)phenyl) piperazin-1-yl)phenyl)-2-naphthamide, N-(4-(4-(4-(((2S,4R)-2-(2,4-dichlorophenyl)-2-methyl-1,3-dioxolan-4-yl) methoxy)phenyl) piperazin-1-yl)phenyl) picolinamide, N-(4-(4-(4-(((2S,4R)-2-(2,4-dichlorophenyl)-2-methyl-1,3-dioxolan-4-yl) methoxy)phenyl) piperazin-1-yl)phenyl) nicotinamide, N-(4-(4-(4-(((2S,4R)-2-(2,4-dichlorophenyl)-2-methyl-1,3-dioxolan-4-yl) methoxy)phenyl) piperazin-1-yl)phenyl) isonicotinamide, N-(4-(4-(4-(((2R,4R)-2-(2,4-dichlorophenyl)-2-methyl-1,3-dioxolan-4-yl) methoxy)phenyl) piperazin-1-yl)phenyl)-2-hydroxybenzamide, N-(4-(4-(4-(((2R,4R)-2-(2,4-dichlorophenyl)-2-methyl-1,3-dioxolan-4-yl) methoxy)phenyl) piperazin-1-yl)phenyl)-3-hydroxybenzamide, N-(4-(4-(4-(((2R,4R)-2-(2,4-dichlorophenyl)-2-methyl-1,3-dioxolan-4-yl) methoxy)phenyl) piperazin-1-yl)phenyl)-4-hydroxybenzamide, N-(4-(4-(4-(((2S,4R)-2-(2,4-dichlorophenyl)-2-methyl-1,3-dioxolan-4-yl) methoxy)phenyl) piperazin-1-yl)phenyl)-2-hydroxybenzamide, N-(4-(4-(4-(((2S,4R)-2-(2,4-dichlorophenyl)-2-methyl-1,3-dioxolan-4-yl) methoxy)phenyl) piperazin-1-yl)phenyl)-3-hydroxybenzamide, N-(4-(4-(4-(((2S,4R)-2-(2,4-dichlorophenyl)-2-methyl-1,3-dioxolan-4-yl) methoxy)phenyl) piperazin-1-yl)phenyl)-4-hydroxybenzamide, or any combination of the foregoing.

In some embodiments, an itraconazole analog comprises one or more substitutions in a meta-position of a phenyl ring, 2S,4R-cis-dioxolane, or a combination thereof.

In some embodiments, an itraconazole analog is N-(4-(4-(4-(((2R,4R)-2-(2,4-dichlorophenyl)-2-methyl-1,3-dioxolan-4-yl) methoxy)phenyl) piperazin-1-yl)phenyl) nicotinamide.

In some embodiments, an itraconazole analog is N-(4-(4-(4-(((2R,4R)-2-(2,4-dichlorophenyl)-2-methyl-1,3-dioxolan-4-yl) methoxy)phenyl) piperazin-1-yl)phenyl)-3-hydroxybenzamide.

In some embodiments, an itraconazole analog is N-(4-(4-(4-(((2S,4R)-2-(2,4-dichlorophenyl)-2-methyl-1,3-dioxolan-4-yl) methoxy)phenyl) piperazin-1-yl)phenyl) nicotinamide.

In some embodiments, an itraconazole analog is N-(4-(4-(4-(((2S,4R)-2-(2,4-dichlorophenyl)-2-methyl-1,3-dioxolan-4-yl) methoxy)phenyl) piperazin-1-yl)phenyl)-3-hydroxybenzamide.

In some embodiments, an agent is an itraconazole analog selected from those listed in Table 2 in Pace et al. (2019).

In some embodiments, an itraconazole analog is an itraconazole stereoisoform. Non-limiting examples of itraconazole analogs include: 3-chloro-N-(4-(4-(4-(((2S,4R)-2-(2,4-dichlorophenyl)-2-methyl-1,3-dioxolan-4-yl) methoxy)phenyl) piperazin-1-yl)phenyl)benzamide, N-(4-(4-(4-(((2S,4R)-2-(2,4-dichlorophenyl)-2-methyl-1,3-dioxolan-4-yl) methoxy)phenyl) piperazin-1-yl)phenyl)-3-methoxybenzamide, 3-acetyl-N-(4-(4-(4-(((2S,4R)-2-(2,4-dichlorophenyl)-2-methyl-1,3-dioxolan-4-yl) methoxy)phenyl) piperazin-1-yl)phenyl)benzamide, N-(4-(4-(4-(((2S,4R)-2-(2,4-dichlorophenyl)-2-methyl-1,3-dioxolan-4-yl) methoxy)phenyl) piperazin-1-yl)phenyl) furan-3-carboxamide, N-(4-(4-(4-(((2S,4R)-2-(2,4-dichlorophenyl)-2-methyl-1,3-dioxolan-4-yl) methoxy)phenyl) piperazin-1-yl)phenyl)thiophene-3-carboxamide, or any combination of the foregoing.

In some embodiments, an itraconazole analog comprises one or more meta-substituted aromatic moiety substitutions, 2S,4R-cis-dioxolane, or a combination thereof.

In some embodiments, an itraconazole analog is 3-acetyl-N-(4-(4-(4-(((2S,4R)-2-(2,4-dichlorophenyl)-2-methyl-1,3-dioxolan-4-yl) methoxy)phenyl) piperazin-1-yl)phenyl)benzamide.

In some embodiments, an agent is an itraconazole analog selected from those listed in Table 1 in Wen et al. (2020).

In some embodiments, an itraconazole analog is an itraconazole stereoisoform. Non-limiting examples of itraconazole analogs include: 2R,4R-Trans-1-(sec-butyl)-4-(4-(4-(4-((2-(2,4-dichlorophenyl)-2-methyl-1,3-dioxolan-4-yl) methoxy)phenyl) piperazin-1-yl)phenyl)-1H-1,2,4-triazol-5 (4H)-one, 2S,4R-Cis-1-(sec-butyl)-4-(4-(4-(4-((2-(2,4-dichlorophenyl)-2-methyl-1,3-dioxolan-4-yl) methoxy)phenyl) piperazin-1-yl)phenyl)-1H-1,2,4-triazol-5 (4H)-one, 2-((R)-sec-Butyl)-4-(4-(4-(4-(((2S,4R)-2-(2,4-dichlorophenyl)-2-(1H-imidazol-1-yl)methyl-1,3-dioxolan-4-yl) methoxy)phenyl) piperazin-1-yl)phenyl)-2,4-dihydro-3H-1,2,4-triazol-3-one, 2-((R)-sec-Butyl)-4-(4-(4-(4-(((2R,4R)-2-(2,4-dichlorophenyl)-2-(1H-imidazol-1-yl)methyl-1,3-dioxolan-4-yl) methoxy)phenyl) piperazin-1-yl)phenyl)-2,4-dihydro-3H-1,2,4-triazol-3-one, 2-((R)-sec-Butyl)-4-(4-(4-(4-(((2S,4R)-2-(2,4-dichlorophenyl)-2-(N-phthalimido-2-yl)methyl-1,3-dioxolan-4-yl) methoxy)phenyl) piperazin-1-yl)phenyl)-2,4-dihydro-3H-1,2,4-triazol-3-one, 2-((R)-sec-Butyl)-4-(4-(4-(4-(((2R,4R)-2-(2,4-dichlorophenyl)-2-(N-phthalimido-2-yl)methyl-1,3-dioxolan-4-yl) methoxy)phenyl) piperazin-1-yl)phenyl)-2,4-dihydro-3H-1,2,4-triazol-3-one, 2-((R)-sec-Butyl)-4-(4-(4-(4-(((2R,4R)-2-(2,4-dichlorophenyl)-1,3-dioxolan-4-yl) methoxy)phenyl) piperazin-1-yl)phenyl)-2,4-dihydro-3H-1,2,4-triazol-3-one, 2-((R)-sec-Butyl)-4-(4-(4-(4-(((2S,4R)-2-(2,4-dichlorophenyl)-1,3-dioxolan-4-yl) methoxy)phenyl) piperazin-1-yl)phenyl)-2,4-dihydro-3H-1,2,4-triazol-3-one, 2-((R)-sec-Butyl)-4-(4-(4-(4-(((2S,4R)-2-(2,4-dichlorophenyl)-2-chloromethyl-1,3-dioxolan-4-y1) methoxy)phenyl) piperazin-1-yl)phenyl)-2,4-dihydro-3H-1,2,4-triazol-3-one, 2-((R)-sec-Butyl)-4-(4-(4-(4-(((2R,4R)-2-(2,4-dichlorophenyl)-2-chloromethyl-1,3-dioxolan-4-yl) methoxy)phenyl) piperazin-1-yl)phenyl)-2,4-dihydro-3H-1,2,4-triazol-3-one, 2-((R)-sec-Butyl)-4-(4-(4-(4-(((2S,4R)-2-(2,4-dichlorophenyl)-2-trifluoromethyl-1,3-dioxolan-4-yl) methoxy)phenyl) piperazin-1-yl)phenyl)-2,4-dihydro-3H-1,2,4-triazol-3-one, 2-((R)-sec-Butyl)-4-(4-(4-(4-(((2R,4R)-2-(2,4-dichlorophenyl)-2-trifluoromethyl-1,3-dioxolan-4-yl) methoxy)phenyl) piperazin-1-yl)phenyl)-2,4-dihydro-3H-1,2,4-triazol-3-one, 2-((R)-sec-Butyl)-4-(4-(4-(4-(((2S,4R)-2-(2,4-dichlorophenyl)-2-carbamoyl-1,3-dioxolan-4-yl) methoxy)phenyl) piperazin-1-yl)phenyl)-2,4-dihydro-3H-1,2,4-triazol-3-one, 2-((R)-sec-Butyl)-4-(4-(4-(4-(((2S,4R)-2-(2,4-dichlorophenyl)-2-aminomethyl-1,3-dioxolan-4-yl) methoxy)phenyl) piperazin-1-yl)phenyl)-2,4-dihydro-3H-1,2,4-triazol-3-one, or any combination of the foregoing.

In some embodiments, an itraconazole analog comprises a modified dioxolane region. In some embodiments, an itraconazole analog comprises an imidazole (e.g., at the position of a triazole in itraconazole), a chloromethyl (e.g., at the position of a methyl group in itraconazole), or a combination thereof.

In some embodiments, an itraconazole analog is 2-((R)-sec-Butyl)-4-(4-(4-(4-(((2S,4R)-2-(2,4-dichlorophenyl)-2-chloromethyl-1,3-dioxolan-4-yl) methoxy)phenyl) piperazin-1-yl)phenyl)-2,4-dihydro-3H-1,2,4-triazol-3-one, 2-((R)-sec-Butyl)-4-(4-(4-(4-(((2R,4R)-2-(2,4-dichlorophenyl)-2-chloromethyl-1,3-dioxolan-4-yl) methoxy)phenyl) piperazin-1-yl)phenyl)-2,4-dihydro-3H-1,2,4-triazol-3-one, 2-((R)-sec-Butyl)-4-(4-(4-(4-(((2S,4R)-2-(2,4-dichlorophenyl)-2-(1H-imidazol-1-yl)methyl-1,3-dioxolan-4-yl) methoxy)phenyl) piperazin-1-yl)phenyl)-2,4-dihydro-3H-1,2,4-triazol-3-one, 2-((R)-sec-Butyl)-4-(4-(4-(4-(((2R,4R)-2-(2,4-dichlorophenyl)-2-(1H-imidazol-1-yl)methyl-1,3-dioxolan-4-yl) methoxy)phenyl) piperazin-1-yl)phenyl)-2,4-dihydro-3H-1,2,4-triazol-3-one, or any combination of the foregoing.

In some embodiments, an agent comprises, consists essentially of, or consists of a polynucleotide (e.g., a short hairpin RNA (shRNA), a single guide RNA (sgRNA), a short interfering RNA (siRNA), a micro RNA (microRNA or miRNA), or an antisense oligonucleotide (oligo)). In some embodiments, an agent comprises a polynucleotide (e.g., a shRNA, a sgRNA, a siRNA, a miRNA, or an antisense oligo). In some embodiments, an agent consists essentially of a polynucleotide (e.g., a shRNA, a sgRNA, a siRNA, a miRNA, or an antisense oligo). In some embodiments, an agent consists of a polynucleotide (e.g., a shRNA, a sgRNA, a siRNA, a miRNA, or an antisense oligo). In some embodiments, an agent is a polynucleotide (e.g., a shRNA, a sgRNA, a siRNA, a miRNA, or an antisense oligo).

In some embodiments, an agent comprises, consists essentially of, or consists of an shRNA targeting C1GALT1. In some embodiments, an agent comprises an shRNA targeting C1GALT1. In some embodiments, an agent consists essentially of an shRNA targeting C1GALT1. In some embodiments, an agent consists of an shRNA targeting C1GALT1. In some embodiments, an agent is an shRNA targeting C1GALT1.

In some embodiments, an agent comprises, consists essentially of, or consists of an sgRNA targeting C1GALT1. In some embodiments, an agent comprises an sgRNA targeting C1GALT1. In some embodiments, an agent consists essentially of an sgRNA targeting C1GALT1. In some embodiments, an agent consists of an sgRNA targeting C1GALT1. In some embodiments, an agent is an sgRNA targeting C1GALT1.

In some embodiments, an agent comprises, consists essentially of, or consists of an siRNA targeting C1GALT1. In some embodiments, an agent comprises an siRNA targeting C1GALT1. In some embodiments, an agent consists essentially of an siRNA targeting C1GALT1. In some embodiments, an agent consists of an siRNA targeting C1GALT1. In some embodiments, an agent is an siRNA targeting C1GALT1.

### Administration

In some embodiments, an agent (e.g., ITZ or an analog thereof) is provided in a liquid form. In some embodiments, an agent (e.g., ITZ or an analog thereof) is provided in a dried form.

In some embodiments, a subject (e.g., a mammalian subject such as a human) has been and/or will be administered an agent (e.g., a therapeutic agent, e.g., ITZ or an analog thereof) according to a method disclosed herein. In some embodiments, a subject has been administered an agent (e.g., ITZ or an analog thereof) according to a method disclosed herein. In some embodiments, a subject will be administered an agent (e.g., ITZ or an analog thereof) according to a method disclosed herein.

The administration of an agent (e.g., in a pharmaceutical composition) may be carried out in any manner, e.g., by parenteral or nonparenteral administration, including by injection, infusion, ingestion, or transfusion, etc. For example, an agent (e.g., ITZ or an analog thereof) may be administered to a subject trans-arterially, intradermally, subcutaneously, intratumorally, or intravenously (e.g., through an intravenous (IV) drip or by intravenous injection). In some embodiments, an agent (e.g., ITZ or an analog thereof) is administered to a subject intravenously. In some embodiments, an agent (e.g., ITZ or an analog thereof) is administered to a subject orally. In some embodiments, an agent (e.g., ITZ or an analog thereof) is administered to a subject intratumorally.

In some embodiments, an agent (e.g., ITZ or an analog thereof) is administered to a subject at about 0.0001-100 mg per kilogram of the body weight of the subject, per day, for example, at about: 0.001-100, 0.01-100, 0.02-100, 0.02-50, 0.05-50, 0.05-20, 0.1-20, 0.2-20, 0.2-10, 0.5-10, 0.5-5, 1-5, 1-3, or 2-3 mg per kilogram of the body weight of the subject, per day. In some embodiments, an agent (e.g., ITZ or an analog thereof) is administered to a subject at about 2 mg per kilogram of the body weight of the subject, per day. In some embodiments, an agent (e.g., ITZ or an analog thereof) is administered to a subject at about 5 mg per kilogram of the body weight of the subject, per day.

In some embodiments, an agent (e.g., ITZ or an analog thereof) is administered to a subject at about 100 mg to about 1,200 mg per day, for example, at about: 100, 150, 200, 250, 300, 350, 400, 450, 500, 550, 600, 700, 800, 900, 1,000, or 1,100 mg per day.

In some embodiments, an agent (e.g., ITZ or an analog thereof) is administered to a subject at about 100 mg to about 1,200 mg, once daily, for example, at about: 100, 150, 200, 250, 300, 350, 400, 450, 500, 550, 600, 700, 800, 900, 1,000, or 1,100 mg, once daily. In some embodiments, an agent (e.g., ITZ or an analog thereof) is administered to a subject at about: 100, 200, 300, 400, 500, or 600 mg, once daily. In some embodiments, an agent (e.g., ITZ or an analog thereof) is administered to a subject at about 200 mg, once daily.

In some embodiments, an agent (e.g., ITZ or an analog thereof) is administered to a subject at about 100 mg to about 1,200 mg, twice daily, for example, at about: 100, 150, 200, 250, 300, 350, 400, 450, 500, 550, 600, 700, 800, 900, 1,000, or 1,100 mg, twice daily. In some embodiments, an agent (e.g., ITZ or an analog thereof) is administered to a subject at about: 100, 200, 300, 400, 500, or 600 mg, twice daily. In some embodiments, an agent is administered to a subject at about 300 mg, twice daily.

In some embodiments, an agent (e.g., ITZ or an analog thereof) is administered (e.g., intravenously or as an oral solution) to a subject at about 10 mg/mL.

In some embodiments, an agent (e.g., ITZ or an analog thereof) is administered to a subject:
a) for at least 1, 2, 3, 4, 5, 6, 7, 8, 9, 10, 11, or 12 weeks,
b) for about 1 day to about 12 weeks, or
both a) and b).

In some embodiments, an agent (e.g., ITZ or an analog thereof) is administered to a subject for at least about 1 week, for example, an agent (e.g., ITZ or an analog thereof) is administered to a subject for at least about: 2, 3, 4, 5, 6, 7, 8, 9, 10, 11, or 12 weeks.

In some embodiments, an agent (e.g., ITZ or an analog thereof) is administered to a subject for about 1 day to about 12 weeks, for example, for about: 1 day to 10 weeks, 1 day to 8 weeks, 1 day to 6 weeks, 1 day to 3 weeks, 1-10 weeks, 1-8 weeks, 1-6 weeks, 1-3 weeks, 2-10 weeks, 2-8 weeks, 2-6 weeks, 2-3 weeks, or 14-17 days.

In some embodiments, an agent (e.g., ITZ or an analog thereof) is administered to a subject on a 21-day cycle.

### Therapeutically Effective Amounts

In some embodiments, a method disclosed herein:
a) reduces expression of Smoothened (SMO);
b) reduces O—N-acetylgalactosamine-glycosylation (O-GalNAc-glycosylation) of SMO;
c) reduces activity of SMO;
d) reduces stability of SMO;
e) reduces ligand-independent hedgehog signaling;
f) reduces expression of GLI family zinc finger 1 (GLI1);
g) reduces expression of GLI family zinc finger 2 (GLI2);
h) reduces binding of GLI1 to EWS RNA binding protein 1 (EWSR1) and Fli-1 proto-oncogene, ETS transcription factor (FLI1) fusion gene (EWSR1::FLI1);
i) reduces binding of GLI2 to EWSR1::FLI1;
j) reduces expression of EWSR1::FLI1;
k) reduces expression of an EWSR1-FLI1 fusion protein (EWSR1::FLI1) target gene;
l) reduces expression of EWSR1 and ETS transcription factor ERG (ERG) fusion gene (EWSR1::ERG);
or any combination of the foregoing (e.g., in a subject).

In some embodiments, a method reduces expression of EWSR1::FLI1, EWSR1::ERG, or both, and:
a) reduces expression of H3 clustered histone 10 (H3C10), zinc finger and BTB domain containing 21 (ZBTB21), histone cell cycle regulator (HIRA), activating transcription factor 6 beta (ATF6B), linker for activation of T cells (LAT), dedicator of cytokinesis 11 (DOCK11), fucosyltransferase 3 (FUT3), 3-hydroxy-3-methylglutaryl-CoA lyase (HMGCL), C1GALT1, C1GALT1-specific chaperone 1 (C1GALT1C1), SMO, GLI1, GLI2 or any combination of the foregoing (e.g., in a subject);
b) reduces activity of H3C10, ZBTB21, HIRA, ATF6B, LAT, DOCK11, FUT3, HMGCL, C1GALT1, C1GALT1C1, SMO, GLI1, GLI2 or any combination of the foregoing (e.g., in a subject);
or both a) and b).

In some embodiments, a method:
reduces expression of C1GALT1;
reduces activity of C1GALT1;
reduces O—N-acetylgalactosamine-glycosylation (i.e., O-GalNAc-glycosylation, O-galactosylation) of Smoothened (SMO);
reduces expression of SMO;
reduces activity of SMO;
reduces ligand-independent hedgehog signaling;
reduces expression of GLI family zinc finger 1 (GLI1,);
reduces binding of GLI1 to EWSR1::FLI1;
reduces expression of EWSR1::FLI1;
reduces expression of an EWSR1::FLI1 target gene;
or any combination of the foregoing (e.g., in a subject).

In some embodiments, a method: reduces expression of GLI family zinc finger 2 (GLI2);
reduces binding of GLI2 to EWSR1::FLI1;
reduces stability of SMO;
reduces expression of EWSR1 and ETS transcription factor ERG (ERG) fusion gene (EWSR1::ERG);
or any combination of the foregoing (e.g., in a subject).

In some embodiments, a therapeutically effective amount is an amount sufficient to:
reduce expression of C1GALT1;
reduce activity of C1GALT1;
reduce O—N-acetylgalactosamine-glycosylation (O-GalNAc-glycosylation) of SMO;
reduce expression of SMO;
reduce activity of SMO;
reduce ligand-independent hedgehog signaling;
reduce expression of GLI1;
reduce binding of GLI1 to EWSR1::FLI1;
reduce expression of EWSR1::FLI1;
reduce expression of an EWSR1::FLI1 target gene;
or any combination of the foregoing (e.g., in a subject).

In some embodiments, a therapeutically effective amount is an amount sufficient to:
reduce expression of GLI family zinc finger 2 (GLI2);
reduce binding of GLI2 to EWSR1::FLI1;
reduce stability of SMO;
reduce expression of EWSR1 and ETS transcription factor ERG (ERG) fusion gene (EWSR1::ERG);
or any combination of the foregoing (e.g., in a subject).

In some embodiments, an amount (e.g., a therapeutically effective amount) of an agent (e.g., ITZ or an analog thereof) or pharmaceutically acceptable salt thereof is sufficient to reduce expression of C1GALT1 in a subject, for example, compared to the same subject, were the subject left untreated. In some embodiments, an amount of an agent (e.g., ITZ or an analog thereof) or pharmaceutically acceptable salt thereof is sufficient to reduce expression of C1GALT1 by at least about 10%, for example, by at least about: 15%, 20%, 25%, 30%, 50%, 75%, 90%, 95%, 98%, or 99%. In some embodiments, an amount of an agent (e.g., ITZ or an analog thereof) or pharmaceutically acceptable salt thereof is sufficient to reduce expression of C1GALT1 by at least about: 10%, 30%, 50%, or 90%.

In some embodiments, an amount (e.g., a therapeutically effective amount) of an agent (e.g., ITZ or an analog thereof) or pharmaceutically acceptable salt thereof is sufficient to reduce activity of C1GALT1 in a subject, for example, compared to the same subject, were the subject left untreated. In some embodiments, an amount of an agent (e.g., ITZ or an analog thereof) or pharmaceutically acceptable salt thereof is sufficient to reduce activity of C1GALT1 by at least about 10%, for example, by at least about: 15%, 20%, 25%, 30%, 50%, 75%, 90%, 95%, 98%, or 99%. In some embodiments, an amount of an agent (e.g., ITZ or an analog thereof) or pharmaceutically acceptable salt thereof is sufficient to reduce activity of C1GALT1 by at least about: 10%, 30%, 50%, or 90%.

In some embodiments, an amount (e.g., a therapeutically effective amount) of an agent (e.g., ITZ or an analog thereof) or pharmaceutically acceptable salt thereof is sufficient to reduce O-GalNAc-glycosylation of Smoothened (SMO) in a subject, for example, compared to the same subject, were the subject left untreated. In some embodiments, an amount of an agent (e.g., ITZ or an analog thereof) or pharmaceutically acceptable salt thereof is sufficient to reduce O-GalNAc-glycosylation of SMO by at least about 10%, for example, by at least about: 15%, 20%, 25%, 30%, 50%, 75%, 90%, 95%, 98%, or 99%. In some embodiments, an amount of an agent (e.g., ITZ or an analog thereof) or pharmaceutically acceptable salt thereof is sufficient to reduce O-GalNAc-glycosylation of SMO by at least about: 10%, 30%, 50%, or 90%.

In some embodiments, an amount (e.g., a therapeutically effective amount) of an agent (e.g., ITZ or an analog thereof) or pharmaceutically acceptable salt thereof is sufficient to reduce expression of SMO in a subject, for example, compared to the same subject, were the subject left untreated. In some embodiments, an amount of an agent (e.g., ITZ or an analog thereof) or pharmaceutically acceptable salt thereof is sufficient to reduce expression of SMO by at least about 10%, for example, by at least about: 15%, 20%, 25%, 30%, 50%, 75%, 90%, 95%, 98%, or 99%. In some embodiments, an amount of an agent (e.g., ITZ or an analog thereof) or pharmaceutically acceptable salt thereof is sufficient to reduce expression of SMO by at least about: 10%, 30%, 50%, or 90%.

In some embodiments, an amount (e.g., a therapeutically effective amount) of an agent (e.g., ITZ or an analog thereof) or pharmaceutically acceptable salt thereof is sufficient to reduce activity of SMO in a subject, for example, compared to the same subject, were the subject left untreated. In some embodiments, an amount of an agent (e.g., ITZ or an analog thereof) or pharmaceutically acceptable salt thereof is sufficient to reduce activity of SMO by at least about 10%, for example, by at least about: 15%, 20%, 25%, 30%, 50%, 75%, 90%, 95%, 98%, or 99%. In some embodiments, an amount of an agent (e.g., ITZ or an analog thereof) or pharmaceutically acceptable salt thereof is sufficient to reduce activity of SMO by at least about: 10%, 30%, 50%, or 90%.

In some embodiments, an amount (e.g., a therapeutically effective amount) of an agent (e.g., ITZ or an analog thereof) or pharmaceutically acceptable salt thereof is sufficient to reduce ligand-independent hedgehog signaling in a subject, for example, compared to the same subject, were the subject left untreated. In some embodiments, an amount of an agent (e.g., ITZ or an analog thereof) or pharmaceutically acceptable salt thereof is sufficient to reduce ligand-independent hedgehog signaling by at least about 10%, for example, by at least about: 15%, 20%, 25%, 30%, 50%, 75%, 90%, 95%, 98%, or 99%. In some embodiments, an amount of an agent (e.g., ITZ or an analog thereof) or pharmaceutically acceptable salt thereof is sufficient to reduce ligand-independent hedgehog signaling by at least about: 10%, 30%, 50%, or 90%.

In some embodiments, an amount (e.g., a therapeutically effective amount) of an agent (e.g., ITZ or an analog thereof) or pharmaceutically acceptable salt thereof is sufficient to reduce expression of GLI1 in a subject, for example, compared to the same subject, were the subject left untreated. In some embodiments, an amount of an agent (e.g., ITZ or an analog thereof) or pharmaceutically acceptable salt thereof is sufficient to reduce expression of GLI1 by at least about 10%, for example, by at least about: 15%, 20%, 25%, 30%, 50%, 75%, 90%, 95%, 98%, or 99%. In some embodiments, an amount of an agent (e.g., ITZ or an analog thereof) or pharmaceutically acceptable salt thereof is sufficient to reduce expression of GLI1 by at least about: 10%, 30%, 50%, or 90%.

In some embodiments, an amount (e.g., a therapeutically effective amount) of an agent (e.g., ITZ or an analog thereof) or pharmaceutically acceptable salt thereof is sufficient to reduce binding of GLI1 to EWSR1::FLI1 in a subject, for example, compared to the same subject, were the subject left untreated. In some embodiments, an amount of an agent (e.g., ITZ or an analog thereof) or pharmaceutically acceptable salt thereof is sufficient to reduce binding of GLI1 to EWSR1::FLI1 by at least about 10%, for example, by at least about: 15%, 20%, 25%, 30%, 50%, 75%, 90%, 95%, 98%, or 99%. In some embodiments, an amount of an agent (e.g., ITZ or an analog thereof) or pharmaceutically acceptable salt thereof is sufficient to reduce binding of GLI1 to EWSR1::FLI1 by at least about: 10%, 30%, 50%, or 90%.

In some embodiments, an amount (e.g., a therapeutically effective amount) of an agent (e.g., ITZ or an analog thereof) or pharmaceutically acceptable salt thereof is sufficient to reduce expression of EWSR1::FLI1 in a subject, for example, compared to the same subject, were the subject left untreated. In some embodiments, an amount of an agent (e.g., ITZ or an analog thereof) or pharmaceutically acceptable salt thereof is sufficient to reduce expression of EWSR1::FLI1 by at least about 10%, for example, by at least about: 15%, 20%, 25%, 30%, 50%, 75%, 90%, 95%, 98%, or 99%. In some embodiments, an amount of an agent (e.g., ITZ or an analog thereof) or pharmaceutically acceptable salt thereof is sufficient to reduce expression of EWSR1::FLI1 by at least about: 10%, 30%, 50%, or 90%.

In some embodiments, an amount (e.g., a therapeutically effective amount) of an agent (e.g., ITZ or an analog thereof) or pharmaceutically acceptable salt thereof is sufficient to reduce expression of an EWSR1::FLI1 target gene in a subject, for example, compared to the same subject, were the subject left untreated. In some embodiments, an amount of an agent (e.g., ITZ or an analog thereof) or pharmaceutically acceptable salt thereof is sufficient to reduce expression of an EWSR1::FLI1 target gene by at least about 10%, for example, by at least about: 15%, 20%, 25%, 30%, 50%, 75%, 90%, 95%, 98%, or 99%. In some embodiments, an amount of an agent (e.g., ITZ or an analog thereof) or pharmaceutically acceptable salt thereof is sufficient to reduce expression of an EWSR1::FLI1 target gene by at least about: 10%, 30%, 50%, or 90%.

In some embodiments, an EWSR1::FLI1 target gene comprises G protein-coupled receptor kinase 5 (GRK5), nuclear receptor subfamily 0 group b member 1 (NR0B1), vaccinia-related kinase 1 (VRK1), cyclin-dependent kinase inhibitor 1A (CDKN1A), fos-related antigen 2 (FOSL2), forkhead box protein 01 (FOXO1), or any combination of the foregoing.

In some embodiments, an amount (e.g., a therapeutically effective amount) of an agent (e.g., ITZ or an analog thereof) or pharmaceutically acceptable salt thereof is sufficient to reduce expression of GLI2 in a subject, for example, compared to the same subject, were the subject left untreated. In some embodiments, an amount of an agent (e.g., ITZ or an analog thereof) or pharmaceutically acceptable salt thereof is sufficient to reduce expression of GLI2 by at least about 10%, for example, by at least about: 15%, 20%, 25%, 30%, 50%, 75%, 90%, 95%, 98%, or 99%. In some embodiments, an amount of an agent (e.g., ITZ or an analog thereof) or pharmaceutically acceptable salt thereof is sufficient to reduce expression of GLI2 by at least about: 10%, 30%, 50%, or 90%.

In some embodiments, an amount (e.g., a therapeutically effective amount) of an agent (e.g., ITZ or an analog thereof) or pharmaceutically acceptable salt thereof is sufficient to reduce binding of GLI2 to EWSR1::FLI1 in a subject, for example, compared to the same subject, were the subject left untreated. In some embodiments, an amount of an agent (e.g., ITZ or an analog thereof) or pharmaceutically acceptable salt thereof is sufficient to reduce binding of GLI2 to EWSR1::FLI1 by at least about 10%, for example, by at least about: 15%, 20%, 25%, 30%, 50%, 75%, 90%, 95%, 98%, or 99%. In some embodiments, an amount of an agent (e.g., ITZ or an analog thereof) or pharmaceutically acceptable salt thereof is sufficient to reduce binding of GLI2 to EWSR1::FLI1 by at least about: 10%, 30%, 50%, or 90%.

In some embodiments, an amount (e.g., a therapeutically effective amount) of an agent (e.g., ITZ or an analog thereof) or pharmaceutically acceptable salt thereof is sufficient to reduce stability of SMO in a subject, for example, compared to the same subject, were the subject left untreated. In some embodiments, an amount of an agent (e.g., ITZ or an analog thereof) or pharmaceutically acceptable salt thereof is sufficient to reduce stability of SMO by at least about 10%, for example, by at least about: 15%, 20%, 25%, 30%, 50%, 75%, 90%, 95%, 98%, or 99%. In some embodiments, an amount of an agent (e.g., ITZ or an analog thereof) or pharmaceutically acceptable salt thereof is sufficient to reduce stability of SMO by at least about: 10%, 30%, 50%, or 90%.

In some embodiments, an amount (e.g., a therapeutically effective amount) of an agent (e.g., ITZ or an analog thereof) or pharmaceutically acceptable salt thereof is sufficient to increase ubiquitination of SMO in a subject, for example, compared to the same subject, were the subject left untreated. In some embodiments, an amount of an agent (e.g., ITZ or an analog thereof) or pharmaceutically acceptable salt thereof is sufficient to increase ubiquitination of SMO by at least about 10%, for example, by at least about: 15%, 20%, 25%, 30%, 50%, 75%, 90%, 95%, 98%, or 99%. In some embodiments, an amount of an agent (e.g., ITZ or an analog thereof) or pharmaceutically acceptable salt thereof is sufficient to increase ubiquitination of SMO by at least about: 10%, 30%, 50%, or 90%.

In some embodiments, an amount (e.g., a therapeutically effective amount) of an agent (e.g., ITZ or an analog thereof) or pharmaceutically acceptable salt thereof is sufficient to reduce expression of EWSR1::ERG in a subject, for example, compared to the same subject, were the subject left untreated. In some embodiments, an amount of an agent (e.g., ITZ or an analog thereof) or pharmaceutically acceptable salt thereof is sufficient to reduce expression of EWSR1::ERG by at least about 10%, for example, by at least about: 15%, 20%, 25%, 30%, 50%, 75%, 90%, 95%, 98%, or 99%. In some embodiments, an amount of an agent (e.g., ITZ or an analog thereof) or pharmaceutically acceptable salt thereof is sufficient to reduce expression of EWSR1::ERG by at least about: 10%, 30%, 50%, or 90%.

In some embodiments, a method disclosed herein reduces tumor size, improves radiological response, prolongs event-free survival, prolongs disease-free survival, prolongs metastasis-free survival, prolongs overall survival, or any combination of the foregoing.

In some embodiments, an amount (e.g., a therapeutically effective amount) of an agent (e.g., ITZ or an analog thereof) or pharmaceutically acceptable salt thereof is sufficient to reduce tumor size in a subject, for example, compared to the same subject, were the subject left untreated. In some embodiments, an amount of an agent (e.g., ITZ or an analog thereof) or pharmaceutically acceptable salt thereof is sufficient to reduce tumor size by at least about 10%, for example, by at least about: 15%, 20%, 25%, 30%, 50%, 75%, 90%, 95%, 98%, or 99%. In some embodiments, an amount of an agent (e.g., ITZ or an analog thereof) or pharmaceutically acceptable salt thereof is sufficient to reduce tumor size by at least about: 10%, 30%, 50%, or 90%.

In some embodiments, an amount (e.g., a therapeutically effective amount) of an agent (e.g., ITZ or an analog thereof) or pharmaceutically acceptable salt thereof is sufficient to improve radiological response in a subject, for example, compared to the same subject, were the subject left untreated.

In some embodiments, an amount (e.g., a therapeutically effective amount) of an agent (e.g., ITZ or an analog thereof) or pharmaceutically acceptable salt thereof is sufficient to prolong event-free survival in a subject, for example, compared to the same subject, were the subject left untreated. In some embodiments, an amount of an agent (e.g., ITZ or an analog thereof) or pharmaceutically acceptable salt thereof is sufficient to prolong event-free survival by at least about 10%, for example, by at least about: 15%, 20%, 25%, 30%, 50%, 75%, 90%, 95%, 98%, 99%, 1-fold, 2-fold, 5-fold, 10-fold, 20-fold, or 50-fold. In some embodiments, an amount of an agent or pharmaceutically acceptable salt thereof is sufficient to prolong event-free survival by at least about: 10%, 30%, 50%, 90%, 1-fold, 2-fold, 5-fold, or 10-fold.

In some embodiments, an amount (e.g., a therapeutically effective amount) of an agent (e.g., ITZ or an analog thereof) or pharmaceutically acceptable salt thereof is sufficient to prolong disease-free survival in a subject, for example, compared to the same subject, were the subject left untreated. In some embodiments, an amount of an agent (e.g., ITZ or an analog thereof) or pharmaceutically acceptable salt thereof is sufficient to prolong disease-free survival by at least about 10%, for example, by at least about: 15%, 20%, 25%, 30%, 50%, 75%, 90%, 95%, 98%, 99%, 1-fold, 2-fold, 5-fold, 10-fold, 20-fold, or 50-fold. In some embodiments, an amount of an agent (e.g., ITZ or an analog thereof) or pharmaceutically acceptable salt thereof is sufficient to prolong disease-free survival by at least about: 10%, 30%, 50%, 90%, 1-fold, 2-fold, 5-fold, or 10-fold.

In some embodiments, an amount (e.g., a therapeutically effective amount) of an agent (e.g., ITZ or an analog thereof) or pharmaceutically acceptable salt thereof is sufficient to prolong metastasis-free survival in a subject, for example, compared to the same subject, were the subject left untreated. In some embodiments, an amount of an agent (e.g., ITZ or an analog thereof) or pharmaceutically acceptable salt thereof is sufficient to prolong metastasis-free survival by at least about 10%, for example, by at least about: 15%, 20%, 25%, 30%, 50%, 75%, 90%, 95%, 98%, 99%, 1-fold, 2-fold, 5-fold, 10-fold, 20-fold, or 50-fold. In some embodiments, an amount of an agent (e.g., ITZ or an analog thereof) or pharmaceutically acceptable salt thereof is sufficient to prolong metastasis-free survival by at least about: 10%, 30%, 50%, 90%, 1-fold, 2-fold, 5-fold, or 10-fold.

In some embodiments, an amount (e.g., a therapeutically effective amount) of an agent (e.g., ITZ or an analog thereof) or pharmaceutically acceptable salt thereof is sufficient to prolong overall survival in a subject, for example, compared to the same subject, were the subject left untreated. In some embodiments, an amount of an agent (e.g., ITZ or an analog thereof) or pharmaceutically acceptable salt thereof is sufficient to prolong overall survival by at least about 10%, for example, by at least about: 15%, 20%, 25%, 30%, 50%, 75%, 90%, 95%, 98%, 99%, 1-fold, 2-fold, 5-fold, 10-fold, 20-fold, or 50-fold. In some embodiments, an amount of an agent (e.g., ITZ or an analog thereof) or pharmaceutically acceptable salt thereof is sufficient to prolong overall survival by at least about: 10%, 30%, 50%, 90%, 1-fold, 2-fold, 5-fold, or 10-fold.

### Additional Treatment

Administration of an agent (e.g., a compound such as itraconazole), composition, or pharmaceutically acceptable salt described herein may be in conjunction with another active ingredient, for example, simultaneously in the same composition, simultaneously in different dosage forms, or sequentially. An agent (e.g., a compound such as itraconazole), composition, or pharmaceutically acceptable salt described herein, and another active ingredient may be formulated in a single combination, multiple combinations, or separate compositions.

In some embodiments, a method disclosed herein further comprises administering to a subject at least one additional (e.g., second) agent (e.g., a chemotherapy), local treatment (e.g., surgery and/or radiotherapy), cell therapy (e.g., stem cell transplantation such as allogenic stem cell transplantation, therapeutic lymphocytes, Gemogenovatucel-T (Vigil), or therapeutic autologous dendritic cells), or any combination thereof.

In some embodiments, a method further comprises administering to a subject an additional (e.g., second) agent. In some embodiments, a method further comprises administering to a subject a local treatment. In some embodiments, a method disclosed herein further comprises administering to a subject a cell therapy. In some embodiments, a method further comprises administering to a subject a chemotherapy. In some embodiments, a method further comprises administering to a subject a radiation therapy.

In some embodiments, an additional (e.g., second) agent is an antibody (e.g., a monoclonal antibody such as an anti-disialoganglioside (anti-GD2) antibody, ganitumab, bevacizumab, robatumumab, cixutumumab, or TB-403), a chemotherapy drug (e.g., vincristine, temozolomide, melphalan, trabectedin, doxorubicin, lurbinectedin, docetaxel, ifosfamide, eribulin, topotecan, cytarabine, cisplatin, etoposide), an alkylating agent (e.g., busulfan, cyclophosphamide), a CDK4/6 inhibitor (e.g., palbociclib, abemaciclib), a tyrosine kinase inhibitor (e.g., anlotinib), a VEGFR2-TIE2 tyrosine kinase inhibitor (e.g., regorafenib), a poly (ADP-ribose) polymerase (PARP) inhibitor (e.g., olaparib), a topoisomerase I inhibitor (e.g., irinotecan), a lysine-specific demethylase 1 (LSD1) inhibitor (e.g., INCB059872), a microtubule destabilizing agent (e.g., TK216), a checkpoint kinase 1 (Chk1) inhibitor (e.g., LY2880070 (LY)), a nucleoside metabolic inhibitor (e.g., gemcitabine), a vasodilator peptide hormone (e.g., adrenomedullin), a growth factor (e.g., granulocyte-colony stimulating factor (G-CSF), filgrastim, pegfilgrastim, or myeloid growth factor), a cytokine (e.g., aldesleukin), liposomal 9-Nitro-20-(S)-Camptothecin (L9-NC), or Zoledronic acid, or any combination of the foregoing.

In some embodiments, an additional (e.g., second) agent is an antibody. In some embodiments, an additional (e.g., second) agent is a chemotherapy drug. In some embodiments, an additional (e.g.e.g., second) agent is an alkylating agent. In some embodiments, an additional (e.g., second) agent is a CDK4/6 inhibitor. In some embodiments, an additional (e.g., second) agent is a tyrosine kinase inhibitor. In some embodiments, an additional (e.g., second) agent is a VEGFR2-TIE2 tyrosine kinase inhibitor. In some embodiments, an additional (e.g., second) agent is a PARP inhibitor. In some embodiments, an additional (e.g., second) agent is a topoisomerase I inhibitor. In some embodiments, an additional (e.g., second) agent is an LSD1 inhibitor. In some embodiments, an additional (e.g., second) agent is a microtubule destabilizing agent. In some embodiments, an additional (e.g., second) agent is a Chk1 inhibitor. In some embodiments, an additional (e.g., second) agent is a nucleoside metabolic inhibitor. In some embodiments, an additional (e.g., second) agent is a vasodilator peptide hormone. In some embodiments, an additional (e.g., second) agent is a growth factor. In some embodiments, an additional (e.g., second) agent is a cytokine.

### Methods of Reducing Expression of EWSR1::FLI1, Expression of EWSR1::ERG, Hedgehog Pathway Activity, or Smoothened (SMO) Activity in Subjects

The disclosure also provides, among other things, methods of reducing expression of EWSR1::FLI1 in a subject, the methods comprising administering an effective amount of an agent (e.g., ITZ or an analog thereof), or a pharmaceutically acceptable salt thereof, to the subject, wherein the agent (e.g., ITZ or an analog thereof) reduces expression and/or activity of H3 clustered histone 10 (H3C10), zinc finger and BTB domain containing 21 (ZBTB21), histone cell cycle regulator (HIRA), activating transcription factor 6 beta (ATF6B), linker for activation of T cells (LAT), dedicator of cytokinesis 11 (DOCK11), fucosyltransferase 3 (FUT3), 3-hydroxy-3-methylglutaryl-CoA lyase (HMGCL), core 1 synthase, glycoprotein-N-acetylgalactosamine 3-beta-galactosyltransferase 1 (C1GALT1), C1GALT1-specific chaperone 1 (C1GALT1C1), Smoothened (SMO), GLI family zinc finger 1 (GLI1), GLI family zinc finger 2 (GLI2), or any combination thereof, thereby reducing expression of EWSR1::FLI1 in the subject.

The disclosure also provides, among other things, methods of reducing expression of EWSR1::FLI1, EWSR1::ERG, or both in a subject, the methods comprising administering an effective amount of an agent (e.g., ITZ or an analog thereof), or a pharmaceutically acceptable salt thereof, to the subject, wherein the agent reduces expression of H3C10, ZBTB21, HIRA, ATF6B, LAT, DOCK11, FUT3, HMGCL, C1GALT1, C1GALT1C1, SMO, GLI1, GLI2, or any combination thereof, thereby reducing expression of EWSR1::FLI1, EWSR1::ERG, or both in the subject.

The disclosure also provides, among other things, methods of reducing expression of EWSR1::FLI1, EWSR1::ERG, or both in a subject, the methods comprising administering an effective amount of an agent (e.g., ITZ or an analog thereof), or a pharmaceutically acceptable salt thereof, to the subject, wherein the agent reduces activity of H3C10, ZBTB21, HIRA, ATF6B, LAT, DOCK11, FUT3, HMGCL, C1GALT1, C1GALT1C1, SMO, GLI1, GLI2, or any combination thereof, thereby reducing expression of EWSR1::FLI1, EWSR1::ERG, or both in the subject.

The disclosure also provides, among other things, methods of reducing expression of EWSR1::FLI1, EWSR1::ERG, or both in a subject, the methods comprising administering an effective amount of an agent (e.g., ITZ or an analog thereof), or a pharmaceutically acceptable salt thereof, to the subject, wherein the agent: reduces expression of H3C10, ZBTB21, HIRA, ATF6B, LAT, DOCK11, FUT3, HMGCL, C1GALT1, C1GALT1C1, SMO, GLI1, GLI2, or any combination thereof; and reduces activity of H3C10, ZBTB21, HIRA, ATF6B, LAT, DOCK11, FUT3, HMGCL, C1GALT1, C1GALT1C1, SMO, GLI1, GLI2, or any combination thereof; thereby reducing expression of EWSR1::FLI1, EWSR1::ERG, or both in the subject.

The disclosure also provides, among other things, methods of reducing hedgehog pathway activity in a subject, the methods comprising administering to the subject an effective amount of an agent (e.g., ITZ or an analog thereof) that reduces expression and/or activity of C1GALT1, or a pharmaceutically acceptable salt thereof, thereby reducing hedgehog pathway activity in the subject.

The disclosure also provides, among other things, methods of reducing Smoothened (SMO) activity in a subject, the methods comprising administering to the subject an effective amount of an agent (e.g., ITZ or an analog thereof) that reduces expression and/or activity of C1GALT1, or a pharmaceutically acceptable salt thereof, thereby reducing SMO activity in the subject.

A subject is any one or more subjects described herein.

An agent is any one or more agents described herein.

Methods of Killing and/or Reducing Proliferation of an Ewing Sarcoma Tumor Cell

The disclosure also provides, among other things, methods of killing and/or reducing proliferation of an Ewing sarcoma tumor cell, the methods comprising contacting the cell with an agent (e.g., ITZ or an analog thereof) or a pharmaceutically acceptable salt thereof that reduces expression and/or activity of C1GALT1.

### Cells

In some embodiments, a cell is from any one of the subjects described herein. In some embodiments, a cell is a human cell.

In some embodiments, a cell is an Ewing sarcoma cell. In some embodiments, a cell is a metastatic Ewing sarcoma cell, a non-metastatic Ewing sarcoma cell, or a localized Ewing sarcoma cell. In some embodiments, a cell is a metastatic Ewing sarcoma cell. In some embodiments, a cell is a non-metastatic Ewing sarcoma cell. In some embodiments, a cell is a localized Ewing sarcoma cell.

In some embodiments, a cell is a relapsed or recurrent Ewing sarcoma cell, a metastatic Ewing sarcoma cell, a non-metastatic Ewing sarcoma cell, a localized Ewing sarcoma cell, an Ewing sarcoma cell from a subject, an Ewing sarcoma cell from a cell line, or a combination of the foregoing. In some embodiments, a cell is a relapsed or recurrent Ewing sarcoma cell. In some embodiments, a cell is an Ewing sarcoma cell from a subject. In some embodiments, a cell is an Ewing sarcoma cell from a cell line. In some embodiments, a cell is an Ewing sarcoma cell from a cell line, for example, A-673.

In some embodiments, a cell is from a cancer patient. In some embodiments, cancer is Ewing sarcoma, pancreatic cancer, ovarian cancer, liver cancer, gastric cancer, prostate cancer, esophageal cancer, or head and neck cancer. In some embodiments, cancer is Ewing sarcoma, ovarian cancer, or pancreatic cancer. In some embodiments, cancer is Ewing sarcoma. In some embodiments, cancer is pancreatic cancer. In some embodiments, cancer is ovarian cancer. In some embodiments, cancer is liver cancer. In some embodiments, cancer is gastric cancer. In some embodiments, cancer is prostate cancer. In some embodiments, cancer is esophageal cancer. In some embodiments, cancer is head and neck cancer.

In some embodiments, cancer is Ewing sarcoma. In some embodiments, Ewing sarcoma is Ewing sarcoma of bone, extraosseous Ewing sarcoma (EOE), peripheral primitive neuroectodermal tumor (pPNET), or Askin tumor.

In some embodiments, Ewing sarcoma (e.g., Ewing sarcoma of bone, extraosseous Ewing sarcoma (EOE), peripheral primitive neuroectodermal tumor (pPNET), or Askin tumor) is non-metastatic, localized, metastatic, advanced, or relapsed or refractory. In some embodiments, Ewing sarcoma is localized. In some embodiments, Ewing sarcoma is metastatic. In some embodiments, Ewing sarcoma is advanced Ewing sarcoma. In some embodiments, Ewing sarcoma is relapsed or refractory. In some embodiments, refractory Ewing sarcoma is first recurrent extracranial Ewing sarcoma.

In some embodiments, a cell is from a subject (e.g., a human) newly diagnosed with Ewing sarcoma, for example, newly diagnosed with non-metastatic Ewing sarcoma, localized Ewing sarcoma, or metastatic Ewing sarcoma. In some embodiments, a cell is from a subject newly diagnosed with non-metastatic Ewing sarcoma. In some embodiments, a cell is from a subject newly diagnosed with localized Ewing sarcoma. In some embodiments, a cell is from a subject newly diagnosed with metastatic Ewing sarcoma.

In some embodiments, a cell is from a subject (e.g., a human) that has or is susceptible to metastatic Ewing sarcoma, relapsed or recurrent Ewing sarcoma, non-metastatic Ewing sarcoma, or localized Ewing sarcoma. In some embodiments, a cell is from a subject that has or is susceptible to metastatic Ewing sarcoma. In some embodiments, a cell is from a subject that has or is susceptible to relapsed or recurrent Ewing sarcoma. In some embodiments, a cell is from a subject that has or is susceptible to non-metastatic Ewing sarcoma. In some embodiments, a cell is from a subject that has or is susceptible to localized Ewing sarcoma.

In some embodiments, a cell is from a subject that has metastatic Ewing sarcoma, relapsed or recurrent Ewing sarcoma, non-metastatic Ewing sarcoma, or localized Ewing sarcoma. In some embodiments, a cell is from a subject that has metastatic Ewing sarcoma. In some embodiments, a cell is from a subject that has relapsed or recurrent Ewing sarcoma. In some embodiments, a cell is from a subject that has non-metastatic Ewing sarcoma. In some embodiments, a cell is from a subject that has localized Ewing sarcoma.

In some embodiments, a cell is from a subject that is susceptible to metastatic Ewing sarcoma, relapsed or recurrent Ewing sarcoma, non-metastatic Ewing sarcoma, or localized Ewing sarcoma. In some embodiments, a cell is from a subject that is susceptible to metastatic Ewing sarcoma. In some embodiments, a cell is from a subject that is susceptible to relapsed or recurrent Ewing sarcoma. In some embodiments, a cell is from a subject that is susceptible to non-metastatic Ewing sarcoma. In some embodiments, a cell is from a subject that is susceptible to localized Ewing sarcoma.

In some embodiments, a cell is from a subject (e.g., a human) that is a high-risk Ewing sarcoma patient.

In some embodiments, a cell is from a subject (e.g., a human) that has one or more oncogenic mutations (e.g., substitution, insertion, deletion, chromosomal rearrangement (e.g., duplication, deletion, inversion, or translocation) and/or aberrations (e.g., aberrant expression of tumorigenic or pro-tumor factors, inactivation of tumor suppressor expression)). In some embodiments, a cell is from a subject (e.g., a human) that has one or more oncogenic fusions. In some embodiments, a cell is from a subject (e.g., a human) that has one or more fusion genes. In some embodiments, a fusion gene is an oncogenic fusion gene. In some embodiments, a cell is from a subject (e.g., a human) that has one or more oncogenic fusion genes.

In some embodiments, a cell has one or more oncogenic mutations (e.g., substitution, insertion, deletion, chromosomal rearrangement (e.g., duplication, deletion, inversion, or translocation) and/or aberrations (e.g., aberrant expression of tumorigenic or pro-tumor factors, inactivation of tumor suppressor expression)). In some embodiments, a cell has one or more oncogenic fusions. In some embodiments, a cell has one or more fusion genes. In some embodiments, a fusion gene is an oncogenic fusion gene. In some embodiments, a cell has one or more oncogenic fusion genes.

In some embodiments, a cell is from a subject (e.g., a human) that has an oncogenic fusion involving EWS RNA binding protein 1 (EWSR1). In some embodiments, a cell is from a subject (e.g., a human) that has a fusion gene involving EWSR1. In some embodiments, a fusion gene involving EWSR1 is expressed in cell from a subject (e.g., a human). In some embodiments, a fusion protein including EWSR1, or a part thereof, is expressed in a cell from a subject (e.g., a human).

In some embodiments, a cell has an oncogenic fusion involving EWS RNA binding protein 1 (EWSR1). In some embodiments, a cell has a fusion gene involving EWSR1. In some embodiments, a fusion gene involving EWSR1 is expressed in a cell. In some embodiments, a fusion protein including EWSR1, or a part thereof, is expressed in a cell.

In some embodiments, a cell comprises EWSR1::FLI1 oncogenic fusion. In some embodiments, a cell has an EWSR1::FLI1 fusion gene. In some embodiments, an EWSR1::FLI fusion gene is expressed in a cell. In some embodiments, an EWSR1::FLI1 fusion protein is expressed in a cell.

In some embodiments, a cell has EWSR1::ERG oncogenic fusion. In some embodiments, a cell has an EWSR1::ERG fusion gene. In some embodiments, an EWSR1::ERG fusion gene is expressed in a cell. In some embodiments, an EWSR1::ERG fusion protein is expressed in a cell.

In some embodiments, a cell is from a subject (e.g., a human) that has EWSR1::FLI1 oncogenic fusion. In some embodiments, a cell is from a subject (e.g., a human) that has an EWSR1::FLI1 fusion gene. In some embodiments, an EWSR1::FLI1 fusion gene is expressed in a cell from a subject (e.g., a human). In some embodiments, an EWSR1::FLI1 fusion protein is expressed in a cell from a subject (e.g., a human).

In some embodiments, a cell is from a subject (e.g., a human) that has EWSR1::ERG oncogenic fusion. In some embodiments, a cell is from a subject (e.g., a human) that has an EWSR1::ERG fusion gene. In some embodiments, an EWSR1::ERG fusion gene is expressed in a cell from a subject (e.g., a human). In some embodiments, an EWSR1::ERG fusion protein is expressed in a cell from a subject (e.g., a human).

### Agents

An agent is any one or more agents described herein.

In some embodiments, a method comprises contacting a cell with at least about 0.1 mM of an agent (e.g., ITZ or an analog thereof), for example, at least about: 0.2, 0.5, 1, 1.5, 2, 2.5, 5, 10, 15, 20, 25, 30, 40, 50, 80, or 100 mM of an agent. In some embodiments, a method comprises contacting the cell with at least about 2 mM of an agent (e.g., ITZ or an analog thereof).

In some embodiments, a method comprises contacting a cell with about 1-250 mM of an agent (e.g., ITZ or an analog thereof), for example, about: 2-250, 5-250, 5-200, 10-200, 10-150, 20-150, 20-100, or 50-100 mM of an agent. In some embodiments, a method comprises contacting the cell with about 20-100 mM of an agent (e.g., ITZ or an analog thereof).

In some embodiments, a method comprises contacting a cell with an agent (e.g., ITZ or an analog thereof) for at least about 0.5 hours, for example, for at least about: 1, 2, 3, 4, 5, 6, 8, 10, 12, 18, 24, 30, 36, 42, 48, 54, 60, 72, 84, 96, 108, or 120 hours. In some embodiments, a method comprises contacting a cell with an agent (e.g., ITZ or an analog thereof) for at least 6 hours.

In some embodiments, a method comprises contacting a cell with an agent (e.g., ITZ or an analog thereof) for about 0.5-180 hours, for example, about: 1-180, 1-168, 2-168, 2-156, 4-156, 4-144, 8-144, 8-132, 12-132, 12-120, 18-120, 18-108, 24-108, 24-96, 36-96, 36-84, 48-84, or 48-72 hours. In some embodiments, a method comprises contacting a cell with an agent (e.g., ITZ or an analog thereof) for about 12 to about 120 hours.

In some embodiments, a method:
reduces expression of C1GALT1;
reduces activity of C1GALT1;
reduces O-GalNAc-glycosylation of Smoothened (SMO);
reduces expression of SMO;
reduces activity of SMO;
reduces ligand-independent hedgehog signaling;
reduces expression of GLI1;
reduces binding of GLI1 to EWSR1::FLI1;
reduces expression of EWSR1::FLI1;
reduces expression of an EWSR1::FLI1 target gene;
or any combination of the foregoing (e.g., in a cell).

In some embodiments, a method: reduces expression of GLI family zinc finger 2 (GLI2);
reduces binding of GLI2 to EWSR1::FLI1;
reduces stability of SMO;
reduces expression of EWSR1::ERG;
or any combination of the foregoing (e.g., in a cell).

In some embodiments, a method reduces expression of C1GALT1 in a cell, for example, compared to the same cell were it not treated (e.g., without contacting an agent (e.g., ITZ or an analog thereof) described herein). In some embodiments, a method reduces expression of C1GALT1 by at least about 10%, for example, by at least about: 15%, 20%, 25%, 30%, 50%, 75%, 90%, 95%, 98%, or 99%. In some embodiments, a method reduces expression of C1GALT1 by at least about: 10%, 30%, 50%, or 90%.

In some embodiments, a method reduces activity of C1GALT1 in a cell, for example, compared to the same cell were it not treated (e.g., without contacting an agent described herein (e.g., ITZ or an analog thereof)). In some embodiments, a method reduces activity of C1GALT1 by at least about 10%, for example, by at least about: 15%, 20%, 25%, 30%, 50%, 75%, 90%, 95%, 98%, or 99%. In some embodiments, a method reduces activity of C1GALT1 by at least about: 10%, 30%, 50%, or 90%.

In some embodiments, a method reduces O-GalNAc-glycosylation of SMO in a cell, for example, compared to the same cell were it not treated (e.g., without contacting an agent (e.g., ITZ or an analog thereof) described herein). In some embodiments, a method reduces O-GalNAc-glycosylation of SMO by at least about 10%, for example, by at least about: 15%, 20%, 25%, 30%, 50%, 75%, 90%, 95%, 98%, or 99%. In some embodiments, a method reduces O-GalNAc-glycosylation of SMO by at least about: 10%, 30%, 50%, or 90%.

In some embodiments, a method reduces expression of SMO in a cell, for example, compared to the same cell were it not treated (e.g., without contacting an agent (e.g., ITZ or an analog thereof) described herein). In some embodiments, a method reduces expression of SMO by at least about 10%, for example, by at least about: 15%, 20%, 25%, 30%, 50%, 75%, 90%, 95%, 98%, or 99%. In some embodiments, a method reduces expression of SMO by at least about: 10%, 30%, 50%, or 90%.

In some embodiments, a method reduces activity of SMO in a cell, for example, compared to the same cell were it not treated (e.g., without contacting an agent described herein (e.g., ITZ or an analog thereof)). In some embodiments, a method reduces activity of SMO by at least about 10%, for example, by at least about: 15%, 20%, 25%, 30%, 50%, 75%, 90%, 95%, 98%, or 99%. In some embodiments, a method reduces activity of SMO by at least about: 10%, 30%, 50%, or 90%.

In some embodiments, a method reduces expression of GLI1 in a cell, for example, compared to the same cell were it not treated (e.g., without contacting an agent (e.g., ITZ or an analog thereof) described herein). In some embodiments, a method reduces expression of GLI1 by at least about 10%, for example, by at least about: 15%, 20%, 25%, 30%, 50%, 75%, 90%, 95%, 98%, or 99%. In some embodiments, a method reduces expression of GLI1 by at least about: 10%, 30%, 50%, or 90%.

In some embodiments, a method reduces binding of GLI1 to EWSR1::FLI1 in a cell, for example, compared to the same cell were it not treated (e.g., without contacting an agent (e.g., ITZ or an analog thereof) described herein). In some embodiments, a method reduces binding of GLI1 to EWSR1::FLI1 by at least about 10%, for example, by at least about: 15%, 20%, 25%, 30%, 50%, 75%, 90%, 95%, 98%, or 99%. In some embodiments, a method reduces binding of GLI1 to EWSR1::FLI1 by at least about: 10%, 30%, 50%, or 90%.

In some embodiments, a method reduces expression of EWSR1::FLI1 in a cell, for example, compared to the same cell were it not treated (e.g., without contacting an agent (e.g., ITZ or an analog thereof) described herein). In some embodiments, a method reduces expression of EWSR1::FLI1 by at least about 10%, for example, by at least about: 15%, 20%, 25%, 30%, 50%, 75%, 90%, 95%, 98%, or 99%. In some embodiments, a method reduces expression of EWSR1::FLI1 by at least about: 10%, 30%, 50%, or 90%.

In some embodiments, a method reduces expression of an EWSR1::FLI1 target gene in a cell, for example, compared to the same cell were it not treated (e.g., without contacting an agent (e.g., ITZ or an analog thereof) described herein). In some embodiments, a method reduces expression of an EWSR1::FLI1 target gene by at least about 10%, for example, by at least about: 15%, 20%, 25%, 30%, 50%, 75%, 90%, 95%, 98%, or 99%. In some embodiments, a method reduces expression of an EWSR1::FLI1 target gene by at least about: 10%, 30%, 50%, or 90%.

In some embodiments, an EWSR1::FLI1 target gene comprises G protein-coupled receptor kinase 5 (GRK5), nuclear receptor subfamily 0 group b member 1 (NR0B1), vaccinia-related kinase 1 (VRK1), cyclin-dependent kinase inhibitor 1A (CDKN1A), fos-related antigen 2 (FOSL2), forkhead box protein 01 (FOXO1), or any combination of the foregoing.

In some embodiments, a method reduces expression of GLI2 in a cell, for example, compared to the same cell were it not treated (e.g., without contacting an agent described herein (e.g., ITZ or an analog thereof)). In some embodiments, a method reduces expression of GLI2 by at least about 10%, for example, by at least about: 15%, 20%, 25%, 30%, 50%, 75%, 90%, 95%, 98%, or 99%. In some embodiments, a method reduces expression of GLI2 by at least about: 10%, 30%, 50%, or 90%.

In some embodiments, a method reduces binding of GLI2 to EWSR1::FLI1 in a cell, for example, compared to the same cell were it not treated (e.g., without contacting an agent described herein (e.g., ITZ or an analog thereof)). In some embodiments, a method reduces binding of GLI2 to EWSR1::FLI1 by at least about 10%, for example, by at least about: 15%, 20%, 25%, 30%, 50%, 75%, 90%, 95%, 98%, or 99%. In some embodiments, a method reduces binding of GLI2 to EWSR1::FLI1 by at least about: 10%, 30%, 50%, or 90%.

In some embodiments, a method reduces stability of SMO in a cell, for example, compared to the same cell were it not treated (e.g., without contacting an agent described herein (e.g., ITZ or an analog thereof)). In some embodiments, a method reduces stability of SMO by at least about 10%, for example, by at least about: 15%, 20%, 25%, 30%, 50%, 75%, 90%, 95%, 98%, or 99%. In some embodiments, a method reduces stability of SMO by at least about: 10%, 30%, 50%, or 90%.

In some embodiments, a method reduces expression of EWSR1::ERG in a cell, for example, compared to the same cell were it not treated (e.g., without contacting an agent described herein (e.g., ITZ or an analog thereof)). In some embodiments, a method reduces expression of EWSR1::ERG by at least about 10%, for example, by at least about: 15%, 20%, 25%, 30%, 50%, 75%, 90%, 95%, 98%, or 99%. In some embodiments, a method reduces expression of EWSR1::ERG by at least about: 10%, 30%, 50%, or 90%.

In some embodiments, a method kills a cell. In some embodiments, a method reduces a population of cells by killing one or more cells, for example, compared to the same population of cells were it not treated (e.g., without contacting an agent described herein (e.g., ITZ or an analog thereof)). In some embodiments, a method reduces a population of cells by at least about 10%, for example, by at least about: 15%, 20%, 25%, 30%, 50%, 75%, 90%, 95%, 98%, or 99%. In some embodiments, a method reduces a population of cells by at least about: 10%, 30%, 50%, or 90%.

In some embodiments, a method reduces cell division, for example, compared to the same cell were it not treated (e.g., without contacting an agent (e.g., ITZ or an analog thereof) described herein). In some embodiments, a method reduces cell division by at least about 10%, for example, by at least about: 15%, 20%, 25%, 30%, 50%, 75%, 90%, 95%, 98%, or 99%. In some embodiments, a method reduces cell division by at least about: 10%, 30%, 50%, or 90%.

In some embodiments, a method reduces EWSR1::FLI1-mediated transformation, for example, compared to the same cell population were it not treated (e.g., without contacting an agent (e.g., ITZ or an analog thereof) described herein). In some embodiments, a method reduces EWSR1::FLI1-mediated transformation by at least about 10%, for example, by at least about: 15%, 20%, 25%, 30%, 50%, 75%, 90%, 95%, 98%, or 99%. In some embodiments, a method reduces EWSR1::FLI1-mediated transformation by at least about: 10%, 30%, 50%, or 90%.

### Additional Agents

In some embodiments, a method further comprises contacting a cell with at least one additional (e.g., second) agent. In some embodiments, an additional (e.g., second) agent is an antibody (e.g., a monoclonal antibody such as an anti-disialoganglioside (anti-GD2) antibody, ganitumab, bevacizumab, robatumumab, cixutumumab, or TB-403), a chemotherapy drug (e.g., vincristine, temozolomide, melphalan, trabectedin, doxorubicin, lurbinectedin, docetaxel, ifosfamide, eribulin, topotecan, cytarabine, cisplatin, etoposide), an alkylating agent (e.g., busulfan, cyclophosphamide), an CDK4/6 inhibitor (e.g., palbociclib, abemaciclib), a tyrosine kinase inhibitor (e.g., anlotinib), a VEGFR2-TIE2 tyrosine kinase inhibitor (e.g., regorafenib), a poly (ADP-ribose) polymerase (PARP) inhibitor (e.g., olaparib), a topoisomerase I inhibitor (e.g., irinotecan), a lysine-specific demethylase 1 (LSD1) inhibitor (e.g., INCB059872), an microtubule destabilizing agent (e.g., TK216), a checkpoint kinase 1 (Chk1) inhibitor (e.g., LY2880070 (LY)), a nucleoside metabolic inhibitor (e.g., gemcitabine), a vasodilator peptide hormone (e.g., adrenomedullin), a growth factor (e.g., granulocyte-colony stimulating factor (G-CSF), filgrastim, pegfilgrastim, or myeloid growth factor), a cytokine (e.g., aldesleukin), liposomal 9-Nitro-20-(S)-Camptothecin (L9-NC), or Zoledronic acid, or any combination of the foregoing.

In some embodiments, an additional (e.g., second) agent is an antibody. In some embodiments, an additional (e.g., second) agent is a chemotherapy drug. In some embodiments, an additional (e.g., second) agent is an alkylating agent. In some embodiments, an additional (e.g., second) agent is a CDK4/6 inhibitor. In some embodiments, an additional (e.g., second) agent is a tyrosine kinase inhibitor. In some embodiments, an additional (e.g., second) agent is a VEGFR2-TIE2 tyrosine kinase inhibitor. In some embodiments, an additional (e.g., second) agent is a PARP inhibitor. In some embodiments, an additional (e.g., second) agent is a topoisomerase I inhibitor. In some embodiments, an additional (e.g., second) agent is a LSD1 inhibitor. In some embodiments, an additional (e.g., second) agent is a microtubule destabilizing agent. In some embodiments, an additional (e.g., second) agent is a Chk1 inhibitor. In some embodiments, an additional (e.g., second) agent is a nucleoside metabolic inhibitor. In some embodiments, an additional (e.g., second) agent is a vasodilator peptide hormone. In some embodiments, an additional (e.g., second) agent is a growth factor. In some embodiments, an additional (e.g., second) agent is a cytokine.

In some embodiments, a method further comprises irradiating a cell.

### Methods of and Compositions for Reducing Expression of EWSR1::FLI1, Expression of EWSR1::ERG, Hedgehog Pathway Activity, or Smoothened (SMO) Activity in Subjects

The disclosure also provides, among other things, methods of reducing expression of EWS-FLI1 in a cell, the methods comprising contacting the cell with an agent that reduces expression and/or activity of H3 clustered histone 10 (H3C10), zinc finger and BTB domain containing 21 (ZBTB21), histone cell cycle regulator (HIRA), activating transcription factor 6 beta (ATF6B), linker for activation of T cells (LAT), dedicator of cytokinesis 11 (DOCK11), fucosyltransferase 3 (FUT3), 3-hydroxy-3-methylglutaryl-CoA lyase (HMGCL), core 1 synthase, glycoprotein-N-acetylgalactosamine 3-beta-galactosyltransferase 1 (C1GALT1), C1GALT1-specific chaperone 1 (C1GALT1C1), Smoothened (SMO), GLI1, or a combination thereof, thereby reducing expression of EWS-FLI1 in the cell.

The disclosure also provides, among other things, methods of reducing hedgehog pathway activity in a cell, the methods comprising contacting the cell with an agent that reduces expression and/or activity of C1GALT1, thereby reducing hedgehog pathway activity in the cell.

The disclosure also provides, among other things, methods of reducing Smoothened (SMO) activity in a cell, the methods comprising contacting the cell with an agent that reduces expression and/or activity of C1GALT1, thereby reducing SMO activity in the cell.

A cell is any one or more cells described herein.

An agent is any one or more agents described herein.

The disclosure also provides, among other things, RNAs (e.g., shRNAs or sgRNAs) capable of reducing expression of a gene. In some embodiments, an RNA comprises at least one sequence set forth in SEQ ID NOs: 57-86.

In some embodiments, an RNA comprises one or more modifications.

In some embodiments, a gene is FLI1, and an RNA (e.g., shRNA) comprises a sequence set forth in SEQ ID NO:57.

In some embodiments, a gene is EWSR1, and an RNA (e.g., shRNA) comprises a sequence set forth in SEQ ID NO:58 or 59.

In some embodiments, a gene is EWSR1, and an RNA (e.g., sgRNA) comprises a sequence set forth in SEQ ID NO:85 or 86.

In some embodiments, a gene is C1GALT1, and an RNA (e.g., shRNA) comprises a sequence set forth in SEQ ID NO:60 or 61.

In some embodiments, a gene is C1GALT1, and an RNA (e.g., sgRNA) comprises a sequence set forth in SEQ ID NO:83 or 84.

In some embodiments, a gene is C1GALT1C1, and an RNA (e.g., shRNA) comprises a sequence set forth in SEQ ID NO:62 or 63.

In some embodiments, a gene is Mus musculus (mouse) C1GALT1 (C1GALT1 (M)), and an RNA (e.g., shRNA) comprises a sequence set forth in SEQ ID NO: 64.

In some embodiments, a gene is Mus musculus (mouse) SMO (SMO (M)), and an RNA (e.g., shRNA) comprises a sequence set forth in SEQ ID NO:65.

In some embodiments, a gene is SMO, and an RNA (e.g., shRNA) comprises a sequence set forth in SEQ ID NO:66.

In some embodiments, a gene is H3C10, and an RNA (e.g., shRNA) comprises a sequence set forth in SEQ ID NO:67 or 68.

In some embodiments, a gene is ZBTB21, and an RNA (e.g., shRNA) comprises a sequence set forth in SEQ ID NO:69 or 70.

In some embodiments, a gene is HIRA, and an RNA (e.g., shRNA) comprises a sequence set forth in SEQ ID NO:71 or 72.

In some embodiments, a gene is ATF6B, and an RNA (e.g., shRNA) comprises a sequence set forth in SEQ ID NO:73 or 74.

In some embodiments, a gene is LAT, and an RNA (e.g., shRNA) comprises a sequence set forth in SEQ ID NO:75 or 76.

In some embodiments, a gene is DOCK11, and an RNA (e.g., shRNA) comprises a sequence set forth in SEQ ID NO:77 or 78.

In some embodiments, a gene is FUT3, and an RNA (e.g., shRNA) comprises a sequence set forth in SEQ ID NO:79 or 80.

In some embodiments, a gene is HMGCL, and an RNA (e.g., shRNA) comprises a sequence set forth in SEQ ID NO:81 or 82.

In some embodiments, a gene is GLI1, and an RNA is an siRNA. In some embodiments, a gene is SMO, and an RNA is an siRNA.

The disclosure also provides, among other things, compositions comprising any one or more of RNAs (e.g., shRNAs) disclosed herein.

The disclosure also provides, among other things, pharmaceutical compositions comprising any one or more of the compositions disclosed herein and a pharmaceutically acceptable carrier, excipient, stabilizer, diluent or tonifier.

The disclosure also provides, among other things, kits comprising a container and, optionally, an instruction for use, wherein the container comprises one or more of the compositions (e.g., pharmaceutical compositions) disclosed herein.

### Embodiments

1. A method of treating Ewing sarcoma in a subject, comprising administering to the subject an effective amount of an agent that reduces expression and/or activity of C1GALT1, or a pharmaceutically acceptable salt thereof, thereby treating Ewing sarcoma in the subject.
2. A method of reducing expression of EWS-FLI1 in a subject, comprising administering an effective amount of an agent, or a pharmaceutically acceptable salt thereof, to the subject, wherein the agent reduces expression and/or activity of H3 clustered histone 10 (H3C10), zinc finger and BTB domain containing 21 (ZBTB21), histone cell cycle regulator (HIRA), activating transcription factor 6 beta (ATF6B), linker for activation of T cells (LAT), dedicator of cytokinesis 11 (DOCK11), fucosyltransferase 3 (FUT3), 3-hydroxy-3-methylglutaryl-CoA lyase (HMGCL), core 1 synthase, glycoprotein-N-acetylgalactosamine 3-beta-galactosyltransferase 1 (C1GALT1), C1GALT1-specific chaperone 1 (C1GALT1C1), Smoothened (SMO), GLI1, or any combination thereof, thereby reducing expression of EWS-FLI1 in the subject.
3 A method of reducing hedgehog pathway activity in a subject, comprising administering to the subject an effective amount of an agent that reduces expression and/or activity of C1GALT1, or a pharmaceutically acceptable salt thereof, thereby reducing hedgehog pathway activity in the subject.
4. A method of reducing Smoothened (SMO) activity in a subject, comprising administering to the subject an effective amount of an agent that reduces expression and/or activity of C1GALT1, or a pharmaceutically acceptable salt thereof, thereby reducing SMO activity in the subject.
5. The method of any one of Embodiments 1-4, wherein the agent reduces expression of C1GALT1.
6. The method of any one of Embodiments 1-5, wherein the agent reduces activity of C1GALT1.
7. The method of any one of Embodiments 1-6, wherein the agent is itraconazole or an analog thereof.
8. The method of any one of Embodiments 1-7, wherein the subject is a mammal.
9. The method of any one of Embodiments 1-8, wherein the subject is a human.
10. The method of any one of Embodiments 1-9, wherein the subject is newly diagnosed with metastatic Ewing sarcoma, non-metastatic Ewing sarcoma, or localized Ewing sarcoma.
11. The method of any one of Embodiments 1-10, wherein the subject has or is susceptible to metastatic Ewing sarcoma, relapsed or recurrent Ewing sarcoma, non-metastatic Ewing sarcoma, or localized Ewing sarcoma.
12. The method of Embodiment 11, wherein the subject has metastatic Ewing sarcoma.
13. The method of Embodiment 11, wherein the subject has relapsed or recurrent Ewing sarcoma.
14. The method of any one of Embodiments 1-13, wherein the subject at diagnosis is <50 years of age.
15. The method of any one of Embodiments 1-14, wherein the subject is between 2 to 50 years of age.
16. The method of any one of Embodiments 1-15, wherein the subject is 40 years of age or younger.
17. The method of any one of Embodiments 1-16, wherein the agent is administered to the subject at about 0.0001 to about 100 mg per kilogram of body weight per day.
18. The method of any one of Embodiments 1-17, wherein the agent is administered to the subject:
   a) at about 100 to about 1,200 mg per day,
   b) at about 100 mg, about 200 mg, about 300 mg, about 400 mg, about 500 mg, or about 600 mg, once daily, or
   c) at about 100 mg, about 200 mg, about 300 mg, about 400 mg, about 500 mg, or about 600 mg, twice daily.
19. The method of any one of Embodiments 1-17, wherein the agent is administered to the subject at about 10 mg/mL.
20. The method of any one of Embodiments 1-19, wherein the agent is administered to the subject:
   a) for at least 1, 2, 3, 4, 5, 6, 7, 8, 9, 10, 11 or 12 weeks,
   b) for about 1 day to about 12 weeks, or
   both a) and b).
21. The method of any one of Embodiments 1-20, wherein the agent is administered intravenously or orally.
22. The method of any one of Embodiments 1-21, further comprising administering to the subject a second agent, a chemotherapy, a radiation therapy, or any combination thereof.
23. The method of any one of Embodiments 1-22, wherein the method:
   a) reduces expression of C1GALT1,
   b) reduces O-galactosylation of Smoothened (SMO),
   c) reduces expression of SMO,
   d) reduces ligand-independent hedgehog signaling,
   e) reduces expression of GLI1,
   f) reduces binding of GLI1 to EWS-FLI1,
   g) reduces expression of EWS-FLI1, or
   h) reduces expression of an EWS-FLI1 target gene,
   or any combination of the foregoing.
24 The method of any one of Embodiments 1-23, wherein the method:
   a) reduces expression of C1GALT1 by at least 10%, at least 30%, at least 50%, or at least 90%,
   b) reduces O-galactosylation of SMO by at least 10%, at least 30%, at least 50%, or at least 90%,
   c) reduces expression of SMO by at least 10%, at least 30%, at least 50%, or at least 90%,
   d) reduces expression of GLI1 by at least 10%, at least 30%, at least 50%, or at least 90%,
   e) reduces binding of GLI1 to EWS-FLI1 by at least 10%, at least 30%, at least 50%, or at least 90%,
   f) reduces expression of EWS-FLI1 by at least 10%, at least 30%, at least 50%, or at least 90%,
   g) reduces expression of an EWS-FLI1 target gene by at least 10%, at least 30%, at least 50%, or at least 90%,
   or any combination of the foregoing.
25. The method of Embodiment 23 or 24, wherein the EWS-FLI1 target gene comprises G protein-coupled receptor kinase 5 (GRK5), nuclear receptor subfamily 0 group b member 1 (NROB1), vaccinia-related kinase 1 (VRK1), cyclin-dependent kinase inhibitor 1A (CDKN1A), fos-related antigen 2 (FOSL2), forkhead box protein 01 (FOXO1), or any combination of the foregoing.
26. The method of any one of Embodiments 1-25, wherein the amount of the agent administered to the subject is sufficient to reduce tumor size, improve radiological response, prolong event free survival, prolong disease free survival, prolong metastasis free survival, prolong overall survival, or any combination of the foregoing.
27. The method of any one of Embodiments 1-26, wherein the amount of the agent administered to the subject is sufficient to reduce tumor size by at least 10%, at least 30%, at least 50%, or at least 90%.
28. A method of killing and/or reducing proliferation of a Ewing sarcoma tumor cell, comprising contacting the cell with an agent that reduces expression and/or activity of C1GALT1.
29 A method of reducing expression of EWS-FLI1 in a cell, comprising contacting the cell with an agent that reduces expression and/or activity of H3 clustered histone 10 (H3C10), zinc finger and BTB domain containing 21 (ZBTB21), histone cell cycle regulator (HIRA), activating transcription factor 6 beta (ATF6B), linker for activation of T cells (LAT), dedicator of cytokinesis 11 (DOCK11), fucosyltransferase 3 (FUT3), 3-hydroxy-3-methylglutaryl-CoA lyase (HMGCL), core 1 synthase, glycoprotein-N-acetylgalactosamine 3-beta-galactosyltransferase 1 (C1GALT1), C1GALT1-specific chaperone 1 (C1GALT1C1), Smoothened (SMO), GLI1, or a combination thereof, thereby reducing expression of EWS-FLI1 in the cell.
30. A method of reducing hedgehog pathway activity in a cell, comprising contacting the cell with an agent that reduces expression and/or activity of C1GALT1, thereby reducing hedgehog pathway activity in the cell.
31. A method of reducing Smoothened (SMO) activity in a cell, comprising contacting the cell with an agent that reduces expression and/or activity of C1GALT1, thereby reducing SMO activity in the cell.
32. The method of any one of Embodiments 28-31, wherein the agent reduces expression of C1GALT1.
33. The method of any one of Embodiments 28-32, wherein the agent reduces activity of C1GALT1.
34. The method of any one of Embodiments 28-33, wherein the agent is itraconazole or an analog thereof.
35. The method of any one of Embodiments 28-34, wherein the cell is a human cell.
36. The method of any one of Embodiments 29-35, wherein the cell is a Ewing sarcoma cell.
37. The method of any one of Embodiments 28-36, wherein the cell is a relapsed or recurrent Ewing sarcoma cell, a metastatic Ewing sarcoma cell, a non-metastatic Ewing sarcoma cell, or a localized Ewing sarcoma cell.
38. The method of any one of Embodiments 28-37, wherein the cell is a metastatic Ewing sarcoma cell.
39. The method of any one of Embodiments 28-38, wherein the cell is from a subject having relapsed or recurrent Ewing sarcoma.
40. The method of any one of Embodiments 28-39, comprising contacting the cell with at least 2 mM of the agent.
41. The method of any one of Embodiments 28-40, comprising contacting the cell with about 20 to about 100 mM of the agent.
42. The method of any one of Embodiments 28-41, comprising contacting the cell with the agent for at least 6 hours.
43. The method of any one of Embodiments 28-42, comprising contacting the cell with the agent for about 12 to about 120 hours.
44. The method of any one of Embodiments 28-43, further comprising contacting the cell with a second agent.
45. The method of any one of Embodiments 28-44, further comprising irradiating the cell.
46 The method of any one of Embodiments 28-45, wherein the method:
   a) reduces expression of C1GALT1,
   b) reduces O-galactosylation of Smoothened (SMO),
   c) reduces expression of SMO,
   d) reduces ligand-independent hedgehog signaling,
   e) reduces expression of GLI1,
   f) reduces binding of GLI1 to EWS-FLI1,
   g) reduces expression of EWS-FLI1, or
   h) reduces expression of an EWS-FLI1 target gene,
   or any combination of the foregoing.
47. The method of any one of Embodiments 28-46, wherein the method:
   a) reduces expression of C1GALT1 by at least 10%, at least 30%, at least 50%, or at least 90%,
   b) reduces O-galactosylation of SMO by at least 10%, at least 30%, at least 50%, or at least 90%,
   c) reduces expression of SMO by at least 10%, at least 30%, at least 50%, or at least 90%,
   d) reduces expression of GLI1 by at least 10%, at least 30%, at least 50%, or at least 90%,
   e) reduces binding of GLI to EWS-FLI1 by at least 10%, at least 30%, at least 50%, or at least 90%,
   f) reduces expression of EWS-FLI1 by at least 10%, at least 30%, at least 50%, or at least 90%,
   g) reduces expression of an EWS-FLI1 target gene by at least 10%, at least 30%, at least 50%, or at least 90%,
   or any combination of the foregoing.
48. The method of Embodiment 46 or 47, wherein the EWS-FLI1 target gene comprises G protein-coupled receptor kinase 5 (GRK5), nuclear receptor subfamily 0 group b member 1 (NROB1), vaccinia-related kinase 1 (VRK1), cyclin-dependent kinase inhibitor 1A (CDKN1A), fos-related antigen 2 (FOSL2), forkhead box protein 01 (FOXO1), or any combination of the foregoing.
49. The method of any one of Embodiments 28-48, wherein the agent reduces cell division.
50. The method of any one of Embodiments 28-49, wherein the agent reduces EWS-FLI1-mediated transformation of the cell.

### Exemplification

The characteristic, defining feature of ES is a somatic chromosomal translocation that fuses a transactivation domain of the RNA-binding protein, EWSR1, with the DNA-binding domain of an ETS family proto-oncoprotein, most commonly FLI1 (90-95% of cases) or, more rarely, ETS transcription factor ERG (ERG) (5-10% of cases) (Riggi et al., 2021). The resultant EWSR1::FLI1 is an oncogenic fusion-protein that can act as either a transcriptional activator or a transcriptional repressor (Cidre-Aranaz and Alonso, 2015). A variety of EWSR1::FLI1 target genes have been identified and it is generally believed that the aberrant transcriptional regulation of these target genes by EWSR1::FLI1 is the basis of transformation (Cidre-Aranaz and Alonso, 2015). Studies have demonstrated a strict requirement of EWSR1::FLI1 for the survival and/or proliferation of ES cells in culture and growth of ES tumors in mice (Castillero-Trejo et al., 2005; Hu-Lieskovan et al., 2005; May et al., 1993; Riggi et al., 2005; Tanaka et al., 1997). This requirement, in conjunction with the absence of the fusion protein in normal cells, makes EWSR1::FLI1 an attractive therapeutic target. Unfortunately, transcription factors such as EWSR1::FLI1 typically lack enzymatic activities and have been considered to be “undruggable” by conventional small molecule inhibitors (Flores and Grohar, 2021). Efforts to directly inhibit EWSR1::FLI1 have largely been unsuccessful (Uren and Toretsky, 2005).

Here, a novel approach for therapeutically targeting EWSR1::FLI1, through inhibition of druggable factors required for EWSR1::FLI1 expression, is described. Using an innovative functional screening approach, C1GALT1, a galactosyltransferase required for the biosynthesis of many O-glycoproteins (Xia et al., 2022), was identified as a factor that promotes EWSR1::FLI1 expression. The studies described below show that C1GALT1 functions by O-glycosylating and stabilizing the pivotal Hedgehog (Hh) signaling component Smoothened (SMO), thereby activating the Hh signaling pathway, which the data show is required for EWSR1::FLI1 expression. Biological or pharmacological inhibition of C1GALT1 reduces SMO levels, decreases EWSR1::FLI1 expression in human ES cell lines resulting in cell death, and suppresses growth of tumors derived from human ES cell lines in conventional subcutaneous xenograft mouse models. These findings reveal a novel mechanism for inhibiting Hh signaling—and, in particular, a novel mechanism for modulating SMO activity, which is a target of FDA-approved Hh inhibitors (Nguyen and Cho, 2022). In addition to modulating EWSR1::FLI1 expression, the approach is applicable to EWSR1::ERG, which shares the EWSR1 promoter and is therefore expected to be transcriptionally regulated by the same mechanism as EWSR1::FLI1. The findings disclosed below have therapeutic implications for treating ES and other cancers associated with high C1GALT1 expression and/or aberrant Hh signaling.

### Example 1. Methods

### Cell Lines and Culture

The following cell lines were obtained from the American Type Culture Collection (ATCC, Manassas, VA): human Ewing sarcoma (ES) cell lines A673 (also known as A-673; ATCC, CRL-1598) and SK-N-MC (ATCC, HTB-10; see Capes-Davis et al., 2010); human osteosarcoma cell lines Saos-2 (ATCC, HTB-85), MG-63 (ATCC, CRL-1427) and U-2 OS (ATCC, CRL-3455); NIH 3T3 mouse embryonic fibroblasts (ATCC, CRL-1658); IMR-90 human lung fibroblasts (ATCC, CCL-186); human ovarian cancer SK-OV-3 cells (ATCC, HTB-77); human pancreatic cancer cell lines PANC-1 (ATCC, CRL-1469). Pa03C cells were obtained from Johns Hopkins University (Baltimore, MD; Penet et al., 2015). Human ES cell lines TC-71, TC-32, and TC-106 were obtained from the Childhood Cancer Repository at Texas Children's Hospital. Human ovarian cancer OVCAR-8 cells were obtained from Creative Biolabs (Shirley, NY, Cat #IOC-ZP305).

A673, NIH 3T3, PANC-1 and Pa03c cell lines were cultured in Dulbecco's Modified Eagle's Medium (DMEM) (HyClone, Logan, Utah, Cat #SH30022.01). TC-71 cells were cultured in Iscove's Modified Dulbecco's Medium (IMDM) (HyClone, Cat #SH30228.01) supplemented with 1× Insulin-Transferrin-Selenium (ITS; 5 μg/ml insulin) (Gibco, Waltham, MA, Cat #12585014), 5 μg/ml transferrin (Sigma, Burlington, MA, Cat #10652202001), 5 ng/ml selenous acid (Sigma, Cat #211176)). SK-N-MC, MG-63 and IMR-90 cells were cultured in Eagle's Minimum Essential Medium (EMEM) (ATCC, Cat #30-2003). Saos-2 and U-2 OS cells were grown in McCoy's 5A Medium (ATCC, Cat #30-2007). SK-OV-3, OVCAR-8, TC-32 and TC-106 cells were grown in RPMI-1640 (Gibco) Medium (Thermo Fisher Scientific, Waltham, MA, Cat #12633012). All media were supplemented with 10% (v/v) fetal bovine serum (FBS) (Atlanta Biologicals, Flowery Branch, GA, Cat #S11550), 100 units/ml penicillin, 100 μg/ml streptomycin (Gibco, Cat #15140122), 1× MEM non-essential amino acids solution (Gibco, Billings, MT, Cat #11140050). All cell lines were cultured at 37° C. and 5% CO₂.

### Expression Plasmids

Lentiviral expression plasmids were obtained from commercial sources for ectopic expression of SMO (pHAGE-SMO, a gift from Gordon Mills and Kenneth Scott; Addgene (Watertown, MA) plasmid #116792; (Ng et al., 2018), EWSR1::FLI1 (pCDH-puro-EWS-FLI1, a gift from Jialiang Wang; Addgene plasmid #102813; (Loganathan et al., 2016), and C1GALT1 (GeneCopoeia, Rockville, MD, Cat #: EX-W0494-Lv120). Control vectors for the SMO, EWSR1::FLI1, and C1GALT1 expression plasmids were pHAGE puro (a gift from Christopher Vakoc; Addgene plasmid #118692; (Lu et al., 2018)), pCDH-CMV-MCS-EF1a-Puro (System Biosciences, Palo Alto, CA, Cat #CD510B-1 and EX-EGFP-Lv120 (GeneCopoeia, Cat #: EX-EGFP-Lv120), respectively. Lentiviral plasmids expressing FLAG-tagged wild-type full-length SMO, FLAG-SMO (T55A), and FLAG-SMO (T500A) were synthesized by GenScript (Piscataway, NJ) in the pGenLenti vector.

Generation of Cell Lines Stably Ectopically Expressing EWSR1::FLI1, SMO, or C1GALT1

To generate cell lines stably ectopically expressing EWSR1::FLI1 (also referred to as EWS-FLI1, EWS::FLI1, or EWSR1-FLI1), TC-71 or NIH 3T3 cells were transduced with the lentiviral expression plasmid pCDH-puro-EWS-FLI1 or, as a control, pCDH-CMV-MCS-EFla-Puro and cells were selected with puromycin for five days. NIH 3T3 cells stably expressing SMO were generated by transduction of pHAGE-SMO or, as a control, pHAGE puro. NIH 3T3 cells stably expressing C1GALT1 were generated by transduction of C1GALT1-expression plasmid EX-W0494-Lv120 or, as a control, EX-EGFP-Lv120. A673 cell lines stably expressing FLAG-tagged wild-type SMO, FLAG-SMO (T55A), or FLAG-SMO (T500A) used lentiviral plasmids synthesized by GenScript in the pGenLenti vector.

Generation of the A673/EWSR1::FLI1^{tdTomato}/Cas9/EGFP Reporter Cell Line

To generate the reporter cell line using CRISPR/Cas9-mediated homology-directed repair, a donor plasmid was constructed as follows. First, tdTomato complementary DNA (cDNA) was excised from plasmid pCSCMV: tdTomato (a gift from Gerhart Ryffel; Addgene plasmid #30530; (Waldner et al., 2009)) and ligated into pGEM7 (Promega, Madison, WI, Cat #P2391), followed by insertion of a DNA fragment encoding T2A (generated by annealing overlapping primers) downstream and in-frame with tdTomato. A Neomycin-SV40 polyA cassette was created by polymerase chain reaction (PCR) amplification of the Neomycin cDNA from plasmid PGKneobpA (a gift from Philippe Soriano; Addgene plasmid #13342; (Soriano et al., 1991)) and PCR amplification of the SV40 polyA terminator from plasmid pSV-β-Galactosidase (Promega, Cat #E1081); the PCR products were denatured and annealed, PCR amplified, digested and ligated downstream of tdTomato-2A to create pTV-tdTomato-2A-Neo-polyA. Sequence correctness of the plasmid was verified by Sanger sequencing. Lastly, 500 bp of the C-terminal end coding sequence (exon 9) and the 3′ untranslated region (UTR) of the FLI1 gene were PCR-amplified (using primer pairs FLI1 C-term and FLI1 3′UTR, respectively; see Table 2) and subcloned into pTV-tdTomato-2A-Neo-polyA using AscI/Nhel and Xhol/NotI sites, respectively.

The donor plasmid (1 μg) was co-transfected into A673 cells, together with 0.2 μg of plasmid pX330-U6-Chimeric_BB-CBh-hSpCas9 (a gift from Feng Zhang; Addgene plasmid #42230; (Cong et al., 2013)) containing a 20 bp single guide RNA (sgRNA) targeting the EWSR1::FLI1 3′ UTR region (FLI1 3′UTR sgRNA, 5′-CGACGGCGCGCCGCAATTCCTCCTGG-3′) (SEQ ID NO:1), using Lipofectamine RNAiMAX Transfection Reagent (Invitrogen, Waltham, MA, Cat #13778100). After 48 hours of co-transfection, cells were selected with 300 μg G418 (Geneticin) for 10 days. tdTomato-positive cells were isolated using a BD FACSAria II cell sorter (BD Biosciences, Franklin Lakes, NJ) and seeded in 96-well plates by serial dilution to isolate single cell clones. The correct insertion site (i.e., the C-terminus of endogenous FLI1 fused to tdTomato) was verified by performing RT-PCR and genomic DNA PCR assays using a forward primer in FLI1 exon 9 and a reverse primer in tdTomato (FLI1 Ex9 and tdTomato, respectively; Table 2), and a single clone (clone 2) was selected for the reporter cell line.

To confirm that knockdown of EWSR1::FLI1 would reduce tdTomato fluorescence, A673/EWSR1::FLI1^{tdTomato} cells (1×10⁵ cells/well in a 6-well plate) were infected with lentiviral particles expressing a non-silencing (NS), EWSR1, or FLI1 short hairpin RNA (shRNA) (Table 3), selected with 2 μg/ml puromycin for 5 days, and analyzed by flow cytometry using a BD LSRII flow cytometer (BD Biosciences). A673/EWSR1::FLI1^{tdTomato} cells were then infected with lentiviral particles expressing the plasmid lentiCas9-Blast (a gift from Feng Zhang; Addgene plasmid #52962; (Sanjana et al., 2014)) and selected with 3 μg/ml blasticidin for 7 days. Cas9 activity was confirmed by transducing A673/EWSR1::FLI1^{tdTomato}/Cas9 cells with the lentiviral vector pXPR_011 (a gift from John Doench and David Root; Addgene plasmid #59702; (Doench et al., 2014)), which carries a puromycin resistance marker and includes an enhanced green fluorescent protein (EGFP, also referred to as GFP) reporter gene and an sgRNA sequence specifically targeting the EGFP gene, followed by selection with 2 μg/ml puromycin for 5 days and analysis of EGFP by flow cytometry. Finally, A673/EWSR1::FLI1^{tdTomato}/Cas9 cells were infected with lentiviral particles expressing the plasmid pLenti CMV EGFP Blast (659-1) (a gift from Eric Campeau and Paul Kaufman; Addgene plasmid #17445 (Campeau et al., 2009)). Cas9 expression was confirmed in A673/EWSR1::FLI1^{tdTomato}/Cas9/EGFP cells by immunoblotting.

### CRISPR/Cas9 Screen

For the genome-wide CRISPR/Cas9 knockout screen, the A673/EWSR1::FLI1^{tdTomato}/Cas9/EGFP reporter cell line was transduced with the Human CRISPR Brunello Knockout Pooled Library (Addgene, Cat #73178), including about 76,441 sgRNAs targeting 19,114 genes at multiplicity-of-infection (MOI) of 0.3. Cells were selected with 2 μg/ml puromycin for 12 days, followed by a 3-day recovery in the absence of puromycin. At least 1×10⁸ cells were sorted by fluorescence-activated cell sorting (FACS), and the population with low tdTomato (tdTomatolow) and high EGFP (EGFPhigh) expression (about 7%) was isolated and expanded. Total genomic DNA was extracted from the sorted and unsorted populations, and 20 μg was used to prepare a next-generation sequencing (NGS) library as previously described (Evers et al., 2016). Illumina next-generation sequencing was performed, and Bowtiel was used to map the reads to the sgRNA sequences in the human Brunello library with the default parameter settings except′-m 1--best-v 2′ (Langmead et al., 2009). Genes were selected for validation and subsequent analyses if there were at least four sgRNAs significantly enriched in the tdTomatolow EGFPhigh population. sgRNAs with a BH-adjusted P value<0.05 (Benjamini and Hochberg, 1995) and odds ratio of at least 2 using Fisher Exact test were considered as significantly enriched. The statistical analysis and generation of figures were carried out using the CRISPRscreen R package, available at https://github.com/LihuaJulieZhu/CRISPRscreen.

shRNA and siRNA Knockdown

For shRNA knockdown, 1×10⁵ cells per well were seeded in 6-well plates and transduced with 500 μl lentivirus particles expressing an shRNA (listed in Table 3 and obtained from Open Biosystems (Huntsville, AL)/Thermo Fisher Scientific through the University of Massachusetts Chan Medical School's RNAi Core Facility) in a total volume of 1 ml of appropriate medium supplemented with 10 μg/ml polybrene. Medium was replaced after overnight incubation to remove polybrene and viral particles and cells were subjected to puromycin selection (2 μg/ml) for 3 days.

For short interfering RNA (siRNA) knockdown, 6×10⁵ A673 cells, at 60% confluency and cultured in Opti-MEM Reduced-Serum Medium (Gibco Cat #31985-070), were transfected with the following siRNAs (obtained from Dharmacon, Lafayette, CO) at 100 nM using Lipofectamine RNAiMAX Transfection Reagent: GLI1-1 (J-003896-05-0005), GLI1-2 (L-003896-00-0005), GLI2-1 (A006468-14-0005), GLI2-2 (A-006468-15-0005), SMO-1 (A-005726-13-0005), SMO-2 (L-005726-00-0005), or a control pool (D-001810-10-05)

Quantitative RT-PCR (qRT-PCR)

Total RNA was isolated from cells using Trizol (Invitrogen, Cat #15596). cDNA was synthesized using Proto Script II reverse transcription kit (New England Biolabs (NEB), Ipswich, MA, Cat #E6560) and real-time PCR reactions were performed using Quant Studio 3 (Applied Biosystems by Thermo Fisher Scientific) using primer sequences listed in Table 2. Gene expression was normalized to that of GAPDH. A control without reverse transcription (no-RT control) was carried out in parallel to rule out the possibility of DNA contamination. The experiments were performed three independent times.

### Immunoblot Analysis

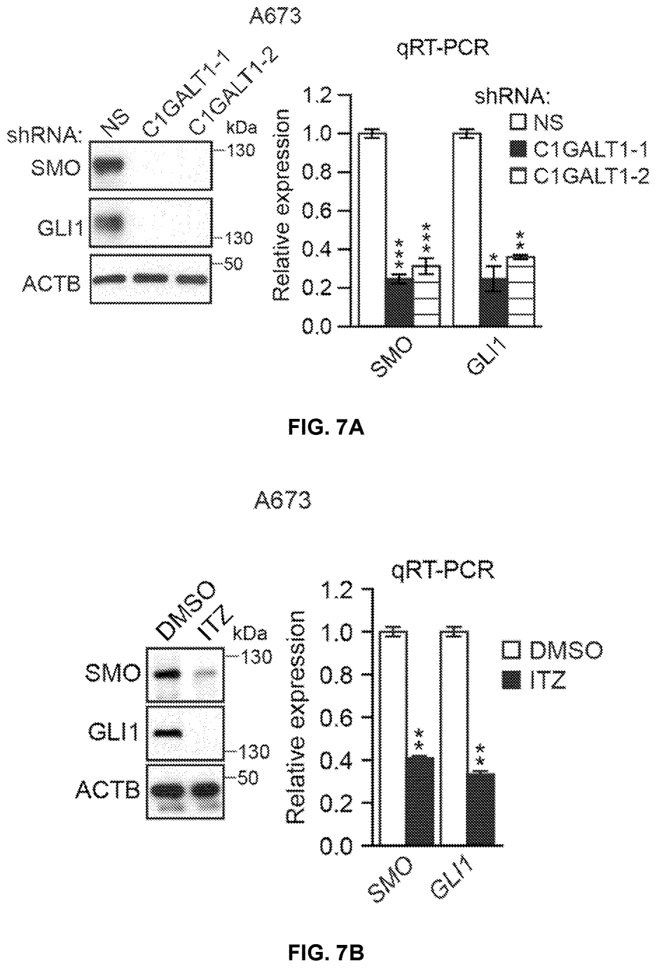

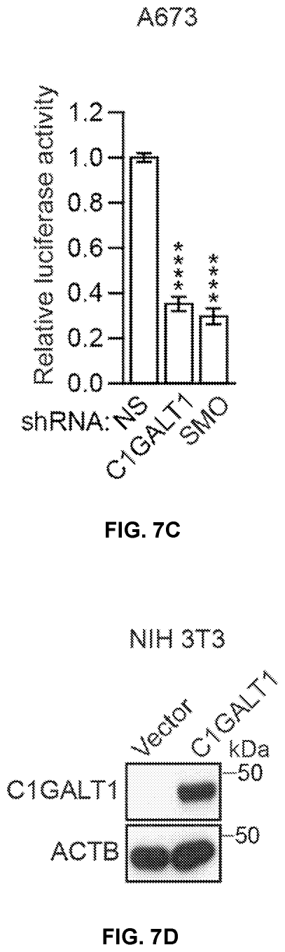

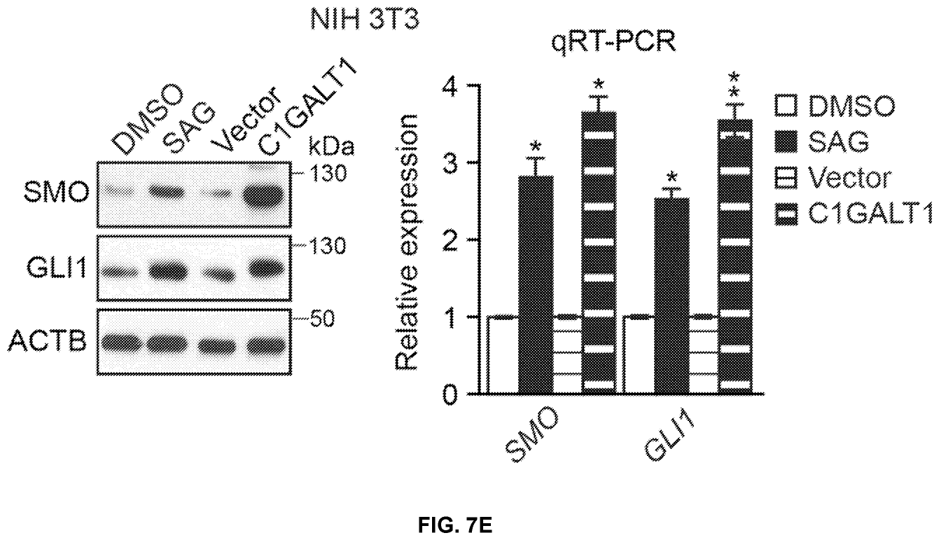

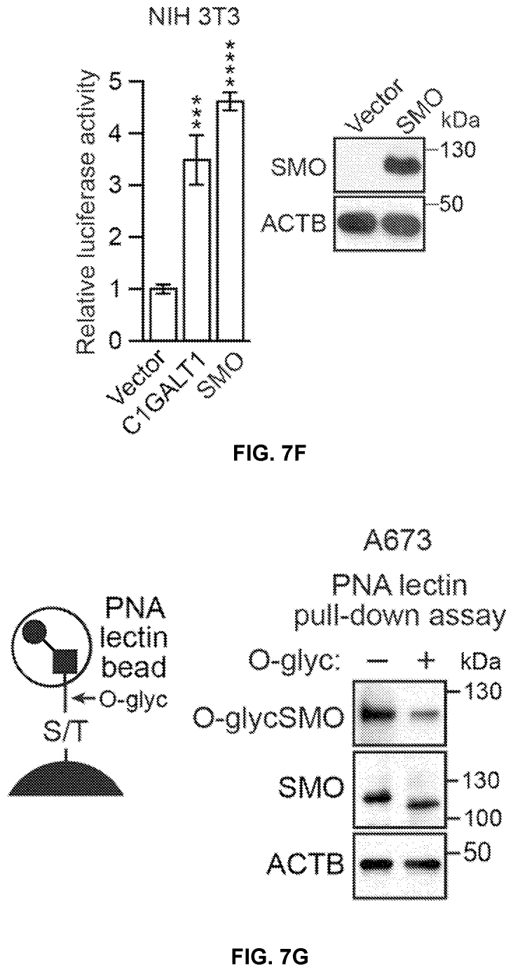

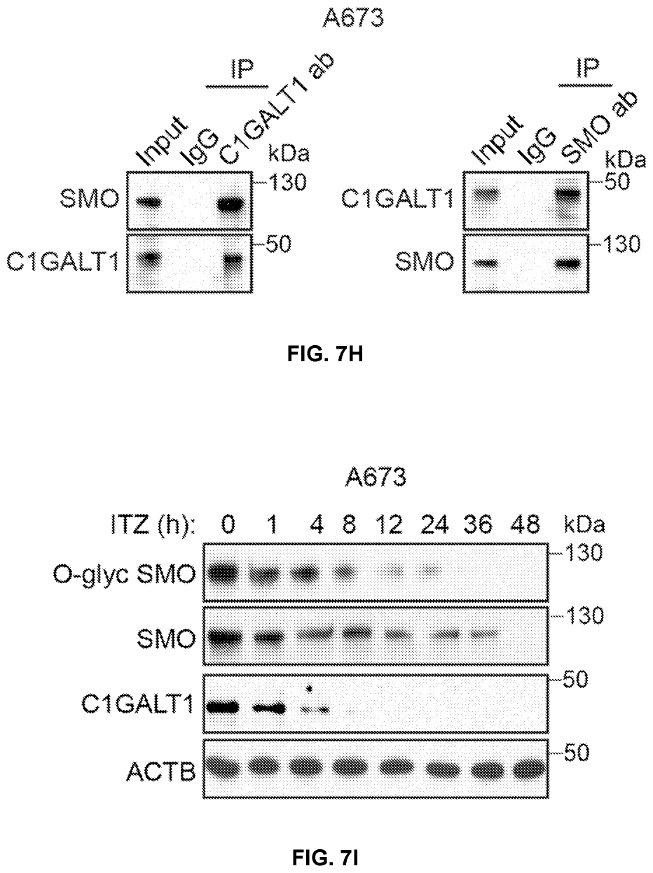

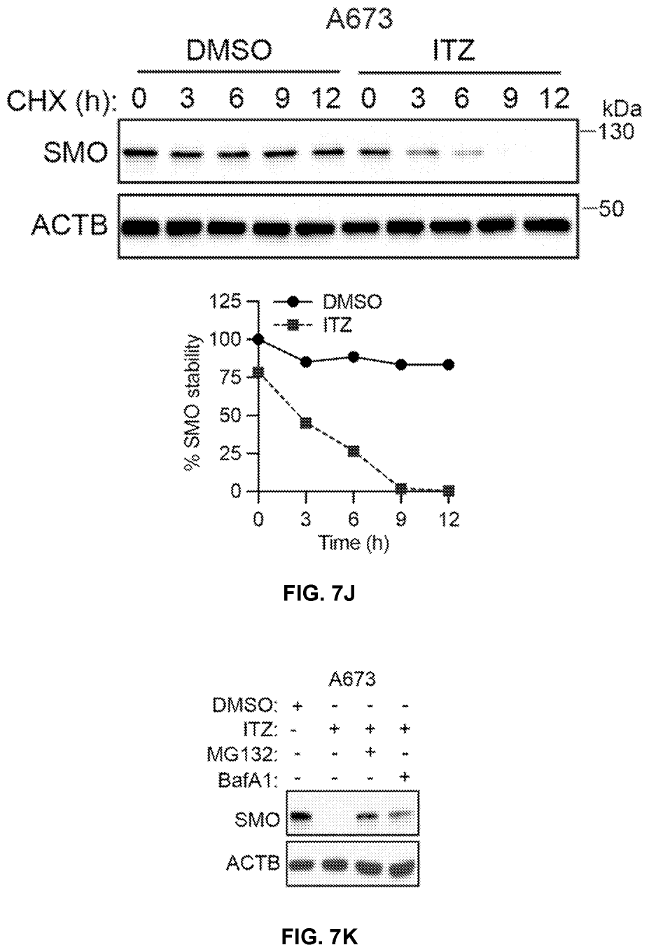

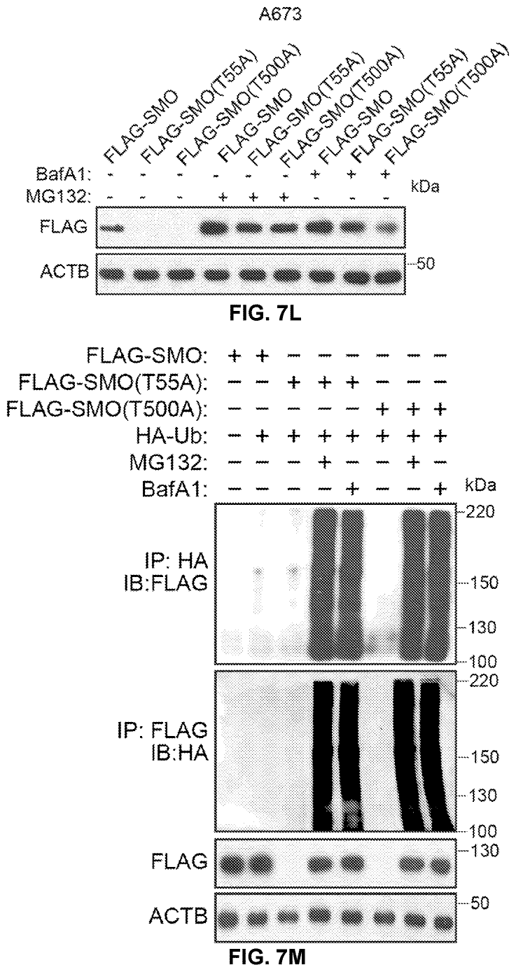

Cells extracts were prepared in radioimmunoprecipitation assay (RIPA) buffer (1% Triton-X100, 0.1% sodium dodecyl sulfate (SDS), 0.5% deoxycholic acid, 20 mM tris(hydroxymethyl)aminomethane (Tris), pH 7.5, 10% glycerol) containing 1× complete ethylenediaminetetraacetic acid (EDTA)-free protease inhibitor cocktail (Roche, Basel, Switzerland, Cat #11873580001) and 1 mM phenylmethylsulfonyl fluoride (PMSF). For the immunoblot in FIG. 7L, cells expressing FLAG-SMO (wild-type and mutants) were treated with 10 μM MG132 (Selleck Chemicals, Houston, TX, Cat #S2619; CAS No. 1211877-36-9) for 12 hours or 100 nM bafilomycin A1 (Selleck Chemicals, Cat #S1413; CAS No. 88899-55-2) for 6 hours. Total cell lysates (30 μg) were subjected to SDS-polyacrylamide gel electrophoresis (PAGE) and transferred to nitrocellulose membrane, which were blocked with 5% non-fat dry milk, and incubated with the following antibodies overnight at 4° C.: anti-C1GALT1 (1:1000 dilution; Santa Cruz Biotechnology, Dallas, TX, Cat #SC-100745), anti-FLI1 (1:2000 dilution; Abcam, Cambridge, United Kingdom, Cat #ab 133485), anti-EWSR1 (1:1000 dilution; Sigma Aldrich, Cat #HPA051771), anti-GLI1 (1:1000 dilution; Cell Signaling Technology, Danvers, MA, Cat #2553), anti-GLI2 (1:500 dilution; Abcam, Cat #ab187386), anti-PARP1 (1:500 dilution; Santa Cruz Biotechnology, Cat #SC-8007), anti-SMO (1:2000 dilution; Abcam, Cat #ab236465), anti-β-actin (1:2000 dilution; Sigma, Cat #A2228), anti-GAPDH (1:2000 dilution; Abcam, Cat #ab8245) or anti-FLAG M2 (1:1000 dilution; Sigma, Cat #F1804). The blots were imaged by exposing to x-ray film. The experiments were conducted three independent times, and the results from one representative experiment are shown.

### Small Molecule Inhibitor Treatment

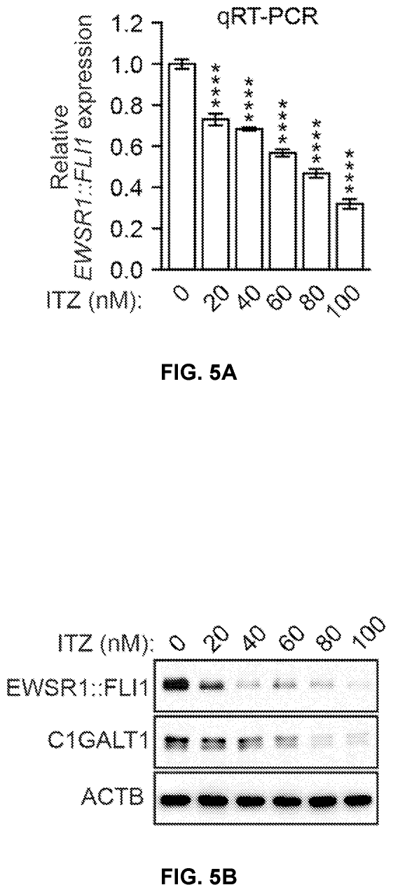

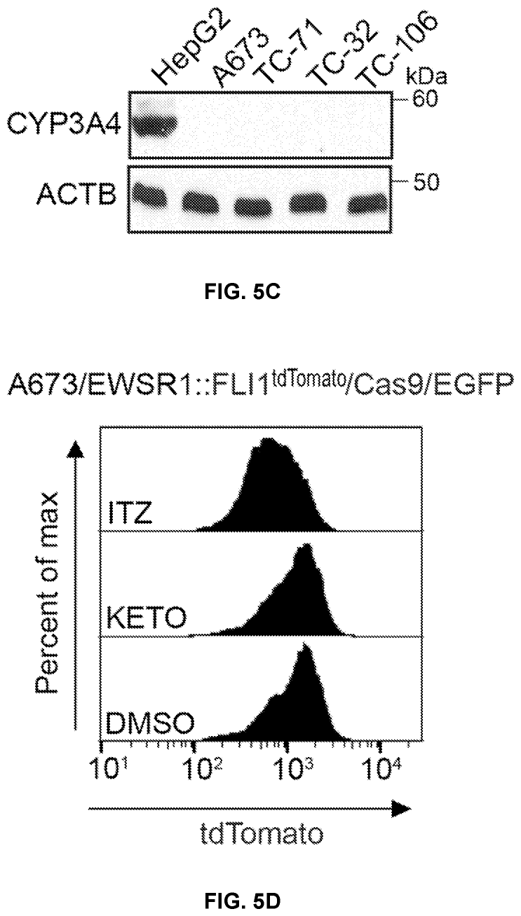

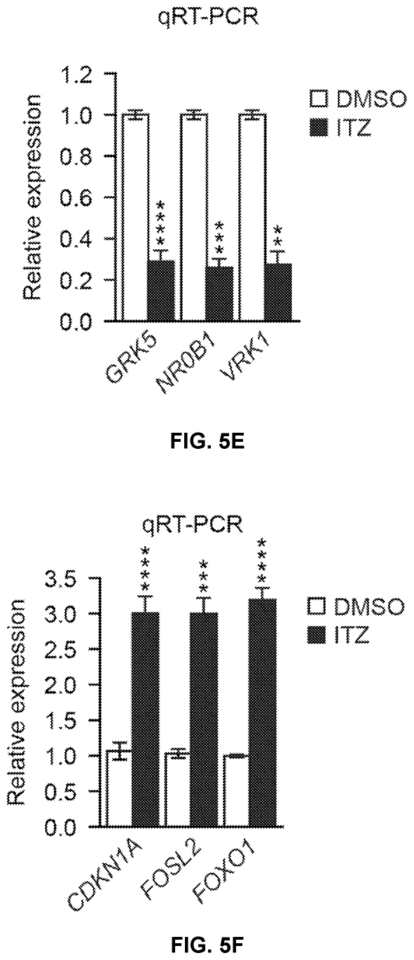

Cells were treated with itraconazole (Sigma-Aldrich, St. Louis, MO, Cat #16657; CAS No. 84625-61-6) at a concentration of 20-100 nM for 72 hours, with SMO inhibitor cyclopamine (Selleck Chemicals, Cat #S1146; CAS No. 4449-51-8), GLI inhibitor GANT-61 (Selleck Chemicals, Cat #S8075; CAS No. 500579-04-4) or Smoothened Agonist hydrochloride (SAG; CAS No. 2095432-58-7) (Selleck Chemicals, Cat #S7779) at a concentration of 2.5-10 μM for 48 hours, or with sonidegib (Selleck Chemicals, Cat #S2151; CAS No. 956697-53-3) or vismodegib (Selleck Chemicals, Cat #S1082; CAS No. 879085-55-9) at a concentration of 1-20 μM for 72 hours. In the experiment in FIG. 5D, cells were treated with 10 μM ketoconazole (Sigma, Cat #PHR1385; CAS No. 65277-42-1) for 72 hours. Dimethyl sulfoxide (DMSO) was used as vehicle control.

### CRISPR Gene Editing

To generate C1GALT1 knockout cell lines, a C1GALT1 sgRNA (sgRNA-1, 5′-GCAACACTTTGTTACAACGC-3′ (SEQ ID NO:83), (Lin et al., 2018); or sgRNA-2, 5′-GAGGTATTCTAACTCATACA-3′ (SEQ ID NO:84), Genscript, Cat #SC1678) was cloned in lentiCRISPR v2 (a gift from Feng Zhang; Addgene plasmid #52961 (Sanjana et al., 2014)). Lentivirus particles expressing the sgRNAs were packaged and used to infect A673 cells. After 48 hours of infection, cells were selected with 2 μg/ml puromycin for 3 days. C1GALT1 knockout was confirmed by immunoblot analysis and Sanger sequencing.

For the deletion of GLI-binding motifs in the EWSR1 promoter, two independent sgRNAs (5′-CGAGACCCTATCCCCGGTAA-3′ (SEQ ID NO:85) and 5′-AACAACTGCTGACTAATCCG-3′ (SEQ ID NO:86)) were designed targeting the flanking regions of GLI-binding sites P2 and P3 and were subcloned into the plasmid lentiCRISPRv2. Lentivirus particles expressing the sgRNAs were packaged and used to infect A673 cells.

After 24 hours of infection, cells were selected with 2 μg/ml puromycin for 3 days and were seeded in 96-well plates by limiting dilution to isolate single cell clones, which were confirmed by Sanger sequencing.

### Chromatin Immunoprecipitation (ChIP) Assays

ChIP assays were performed as described previously (Wajapeyee et al., 2013). In brief, A673 cells treated with 100 nM itraconazole or DMSO were crosslinked with 1% of paraformaldehyde. Fixed cells were suspended in RIPA lysis buffer followed by sonication, using a BIORUPTOR® sonication device (Diagenode, Liege, Belgium). Sonicated chromatin was incubated with 5 μg anti-GLI1 antibody (R&D Systems, Minneapolis, MN, Cat #: AF3324) overnight to immunoprecipitate GLI1. ChIP products were analyzed by qRT-PCR using primers listed in Table 2. Samples were normalized to inputs. Fold enrichment was calculated by setting the IgG control immunoprecipitate (to a value of 1). The experiments were performed three independent times.

### GLI Reporter Assay

NIH 3T3 or A673 cells were co-transfected with 500 ng PGL4.26-10X-GLI1-Luc (generated by Genescript by inserting 10 GLI-binding sites (CCACCCA, corresponding to the sequence found in the EWSR1::FLI1 promoter) into the Nhel/Xhol sites of vector pGL4.26-Luc (Promega, Cat #E844A)) and 50 ng of Renilla luciferase control plasmid (pGL4.72; Promega, Cat #E690A). After 48 hours of co-transfection, cells were lysed with Passive Lysis buffer (Promega, Cat #E1941). Firefly luciferase and Renilla activities were measured using a GlowMax plate reader (Promega) using the Dual-Luciferase Reporter Assay System (Promega, Cat #E1910). The experiments were performed three independent times.

O—N-acetylgalactosamine-glycosylation (O-GalNAc-glycosylation or O-Glycosylation) Assays

For the experiment shown in FIG. 7G, A673 whole cell lysates were treated with 100 U α2-3,6,8 Neuraminidase (NEB, Cat #P0720S) in the presence or absence of 800 U O-glycosidase (NEB, Cat #P0733S) for 2 hours at room temperature prior to SDS-PAGE and immunoblotting with an anti-SMO antibody. For the peanut agglutinin (PNA) lectin pull-down assay of FIG. 7G, 600 μg of total protein from A673 whole cell lysates, treated with neuraminidase in the presence or absence of O-glycosidase as described above, were incubated at 4° C. overnight with peanut agglutinin (PNA)-conjugated agarose beads (Vector Laboratories, Newark, CA, Cat #AL-1073). The beads were washed three times with phosphate buffered saline (PBS) and heated at 95° C. for 10 minutes. The samples were separated by SDS-PAGE and immunoblotted using an anti-SMO antibody. The experiments were performed three independent times.

### Co-immunoprecipitation

A673 cell lysate (200 μg) was incubated with 5 μg anti-SMO antibody, anti-C1GALT1 antibody, or control rabbit or mouse IgG antibody overnight at 4° C. Immune complexes were captured with Magnetic Protein A or G beads (Promega, Cat #G8781 or G7471, respectively) for 45 minutes with gentle rocking at 4° C. The beads were washed twice in lysis buffer, and the associated proteins were extracted in 2× sample buffer (2% w/v SDS, 2 mM dithiothreitol (DTT), 4% v/v glycerol, 0.04 M Tris-HCl, pH 6.8, and 0.01% w/v Bromophenol blue) and analyzed by immunoblotting. Input lanes represent 10% of immunoprecipitated lanes. The experiments were performed three independent times, and the results from one representative experiment are shown.

### Cycloheximide Chase Assays

A673 cells were seeded at a density of 6×10⁵ cells per well in a 6-well plate and grown in DMEM. On the day of the experiment, the medium was replaced with fresh DMEM, and cycloheximide (30 μg/ml) (Thermo Fisher Scientific, Cat #J66004.XF) was added in the presence or absence of 100 nM itraconazole (ITZ) for 3, 6, 9, or 12 hours. DMSO-treated cells served as a control. Densitometry of the bands in the immunoblot was determined using Image J software version 1.47v (National Institutes of Health).

### Ubiquitination Immunoprecipitation Assays

A673 cells stably expressing FLAG-tagged wild-type SMO, FLAG-SMO (T55A) or FLAG-SMO (T500A) were transfected with a plasmid expressing HA-tagged ubiquitin (HA-Ub; Addgene plasmid #18712 (Kamitani et al., 1997) and treated in the presence or absence of 10 μM MG132 for 8 hours or 100 nM bafilomycin A1 for 8 hours. Cell lysates were incubated with either an anti-HA magnetic beads (Sigma, Cat #SAE0197) or anti-FLAG M2 magnetic beads (Sigma, Cat #M8823), and immunoprecipitated proteins were detected using an anti-FLAG M2 (1:1500 dilution; Sigma, Cat #F1804) or anti-HA (1:1500 dilution; Cell Signaling Cat #2367) antibody.

### Soft Agar Colony Formation Assay

A673 cells (1×10⁴) expressing an NS, EWSR1, C1GALT1, or SMO shRNA, or NIH 3T3 cells (5×10³) ectopically expressing empty/control vector, SMO, C1GALT1, or EWSR1::FLI1 were re-suspended in 2 ml of top agar (DMEM containing 0.3% Difco Noble agar (BD Biosciences, Cat #214220)) pre-warmed to 40° C. The cell suspension was layered onto 2 ml of set bottom agar (DMEM containing 0.7% Noble agar) in a 6-well plate. The following day, 1 ml of DMEM was added and was changed every other day. After 3-4 weeks, colonies were stained with 0.05% methylene blue and counted under a microscope. The experiments were performed three independent times.

### Cell Viability Assays

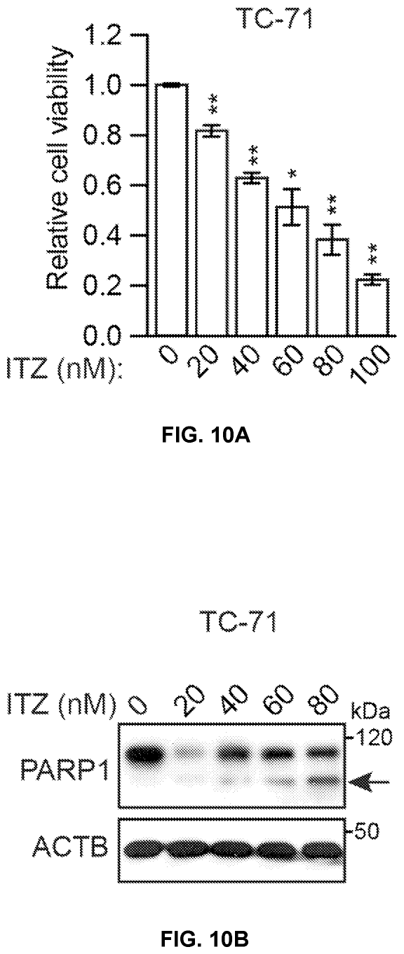

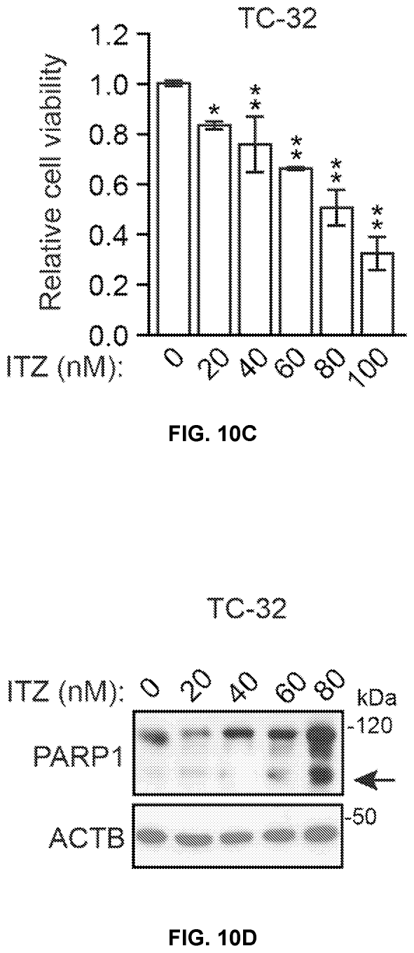

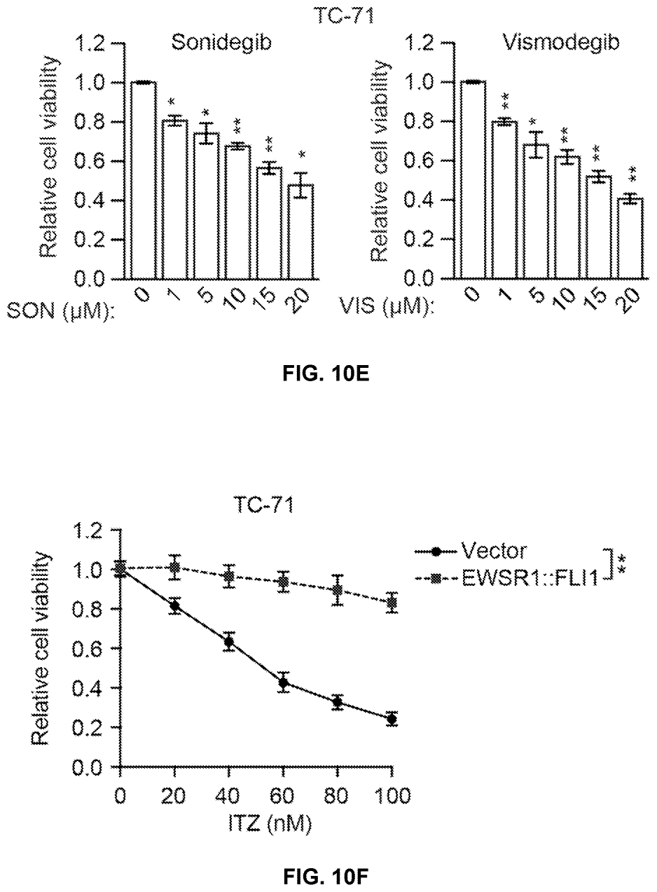

TC-71 and TC-32 cells were seeded in a 96-well plate (5×10³ cells per well), and the following day, ITZ was added at a concentration of 20, 40, 60, 80, or 100 nM for 72 hours. DMSO was used as a vehicle control. For the experiment in FIG. 10F, TC-71 cells stably expressing EWSR1::FLI1 under the control of the CMV promoter were treated with 100 nM ITZ for 72 hours. The cells were then incubated with PRESTOBLUE® Cell Viability Reagent (Invitrogen, Cat #A13261) according to the manufacturer's protocol, and the fluorescence was quantified using a GlowMax Discover Microplate Reader (Promega). The experiments were performed three independent times.

### Tumor Formation Experiments

All mouse studies were performed in accordance with the Guide for the Care and Use of Laboratory Animals from NIH, and protocols (PROTO202000105) approved by the University of Massachusetts Chan Medical School Institutional Animal Care and Use Committee (IACUC). Mice were randomly allocated to each group. No blinding was done as animal groups were identified by tagging and labeling the cages with the cells injected. Animals were excluded, according to pre-established criteria, if the tumor volume reached >2000 mm³; if tumor size or location affected the mobility or general health of animal, the animal was euthanized and excluded from the experiment, or the complete experiment was terminated.

A673, TC-71, TC-106, or TC-32 cells (5×10⁶) were suspended in PBS and MATRIGEL® extracellular matrix (Corning Cat #356234) in a 1:1 ratio, and subcutaneously injected into the right flank of 6-7 week old NOD scid gamma (NSG) immunocompromised mice (n=3 for each vehicle or drug treatment). Tumor dimensions were measured every 3 days with calipers, and the tumor volume was calculated using the formula volume=width²× length/2. When the tumor size reached about 80-100 mm³, itraconazole or vehicle (10% DMSO+40% polyethylene glycol 300 (PEG300; CAS No. 25322-68-3)+5% TWEEN®-80 non-ionic detergent (CAS No. 9005-65-6)+45% Saline (PBS)) was orally administered at 100 mg per kg body weight per day (mg/kg/day) for the duration of the experiment as indicated. For the ITZ withdrawal experiment, tumor-bearing mice were treated for 18 days with ITZ, and then drug treatment was stopped and tumor growth was monitored for an additional 15 days.

### Immunohistochemistry

Bone and cartilage malignant tumor tissue arrays containing formalin-fixed paraffin-embedded 5 μm sections of two human ES patient tumor samples (Biomax, Derwood, MD, Cat #T264a) and one sample each of normal bone and cartilage tissue (Biomax, Cat #T261b) were stained with an anti-C1GALT1 (1:250 Abcam, Cat #ab237734) antibody. Tumor histology was assessed by hematoxylin and eosin (H&E) staining. For the mouse experiments, tumor xenografts were harvested, fixed in 10% buffered formalin phosphate (Fisher Scientific, Waltham, MA, Cat #SF1004) overnight, and then embedded in paraffin blocks. Blocks were sectioned at 4-μm thickness and mounted onto slides, which were stained with an anti-FLI1 (1:500 dilution; Abcam, Cat #ab133485), anti-EWSR1 (1:50 dilution; Sigma Aldrich, Cat #HPA051771), anti-C1GALT1 (1:250 Abcam, Cat #ab 237734), anti-SMO (1:25 dilution; Abcam, Cat #ab236465), anti-GLI1 (1:25; ThermoFisher, Cat #MA5-26639), or anti-Ki67 (1:250; Sino Biological, Beijing, China, Cat #100130-MM22) antibody.

### Bioinformatic Analysis

In-silico GLI motif analysis on the EWSR1 promoter was done using the Broad Institute's Integrative Genomics Viewer (IGV) genome browser (http://software.broadinstitute.org/software/igv/) using the GLI1 consensus sequence GACCACCCA (Gurdziel et al., 2016; Kinzler and Vogelstein, 1990). For Ewing's sarcoma data analysis and Kaplan-Meier plots, datasets GSE17679 and GSE63157_core_transcriptom were downloaded from R2 platform [R2: GenomicsAnalysis and Visualization Platform (http://r2.amc.nl)]. All samples in each dataset were grouped into two groups based on value of the median, average, first quartile, last quartile, and first_vs_last_quartile of the expression of C1GALT1. In the order of expression of C1GALT1, a cutoff with the highest value from log-rank test was employed to make all samples into two groups (scan method). Analysis of overall survival between high and low C1GALT1 expression groups was performed with the R package survival (Therneau, 2023; Therneau and Grambsch, 2000). Kaplan-Meier curves were plotted with R packages survminer (Kassambara et al., 2021) and patchwork (Pedersen, 2022). P values were calculated using the log-rank test.

### Statistics

To achieve statistical significance, all qRT-PCR, ChIP, luciferase reporter, cell viability, and colony formation assays were collected from three independent biological experiments (and for qRT-PCR and ChIP, each independent experiment included at least 3 technical replicates of the sample). The differences between groups were assessed using an unpaired, two-sample student t-test using Prism 10 (GraphPad Software, Boston, MA). Significant differences were considered when P values were less than 0.05 (*P<0.05; **P<0.01, ***P<0.001, and ****P<0.0001). Error bars indicate the standard deviation (SD) or standard error of the mean (SEM), as indicated in BRIEF DESCRIPTION OF THE DRAWINGS, for the replicates.

### Example 2. A Genome-Scale CRISPR/Cas9 Screen Identified Factors that Promote EWSR1::FLI1 Expression

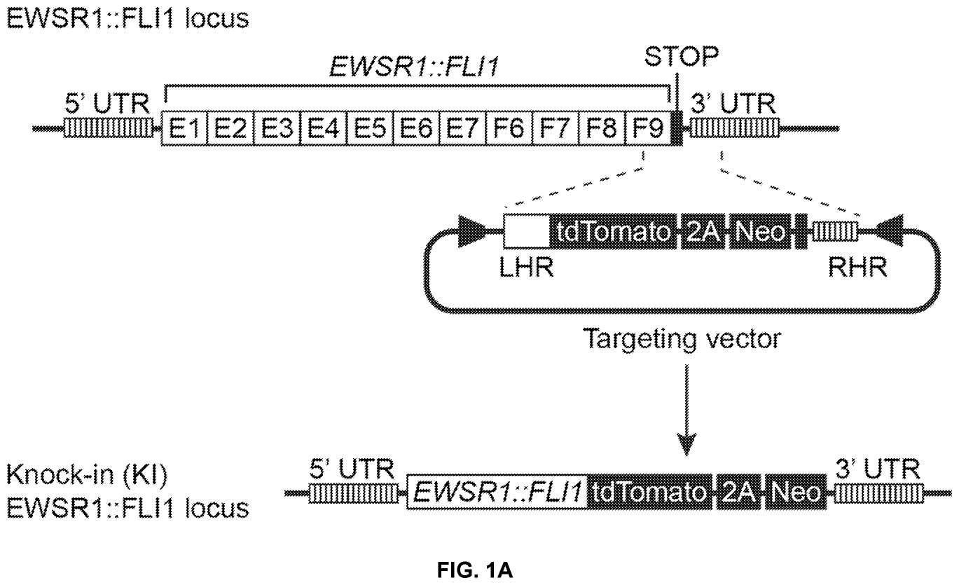

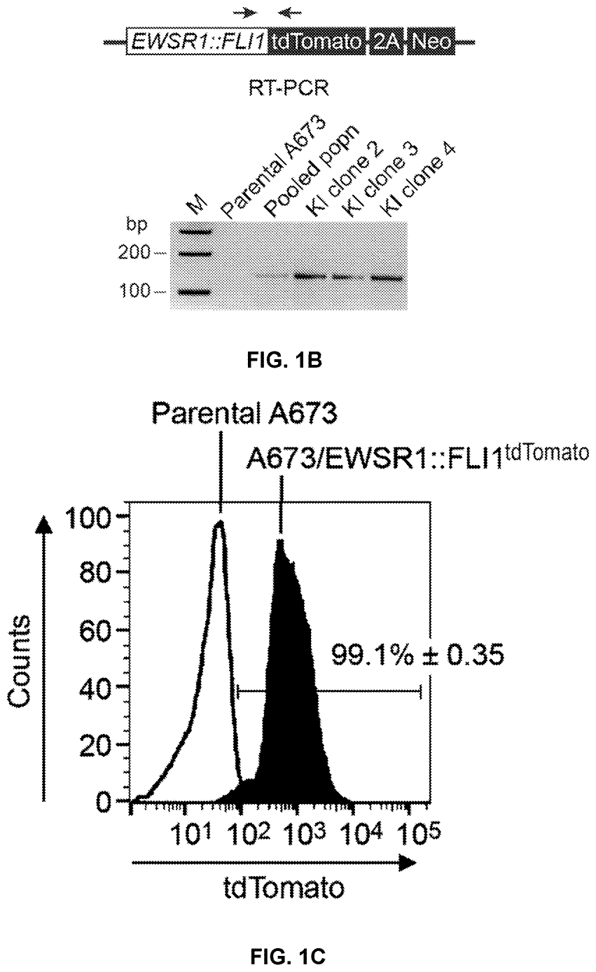

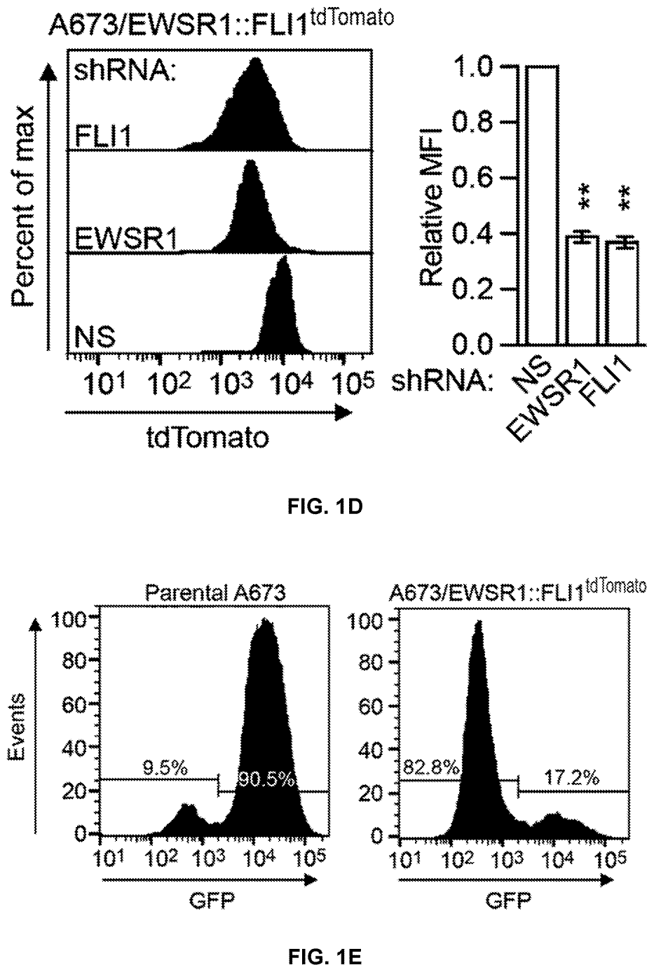

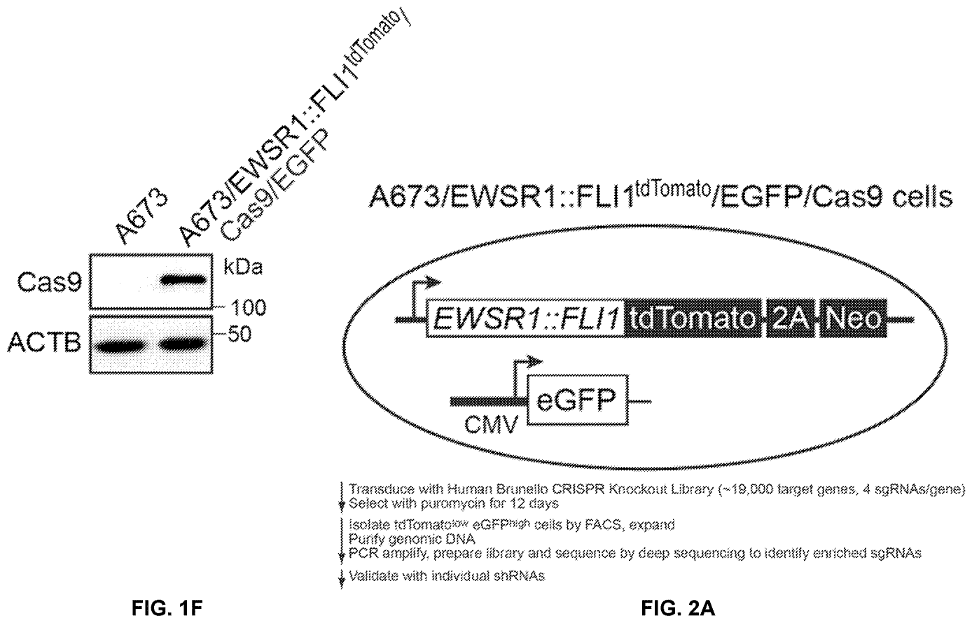

To identify factors that promote EWSR1::FLI1 expression, a genome-scale CRISPR/Cas9-based screen for genes whose knockout reduce EWSR1::FLI1 levels was performed. To facilitate high-throughput screening, a first-of-its-kind EWSR1::FLI1 reporter cell line, in which the endogenous EWSR1::FLI1 gene was fused to the fluorescent reporter tdTomato, was constructed (FIGS. 1A-1D). The reporter cell line was derived from the ES cell line A673, which requires EWSR1::FLI1 to form tumors in mice but not for viability and proliferation in culture and therefore, unlike most other ES cell lines, is not killed following loss of EWSR1::FLI1 (Smith et al., 2006). The reporter cell line also stably expressed active Cas9 (FIGS. 1E-1F) as well as a control fluorophore, enhanced green fluorescence protein (EGFP), driven by a constitutive cytomegalovirus (CMV) promoter, enabling factors whose knockout leads to a general reduction in protein levels or cell survival, which would result in decreased EGFP signal, to be excluded. Cells harboring a gene knockout that selectively reduces expression of EWSR1::FLI1 are identified as tdTomato^{low} EGFP^{high}.

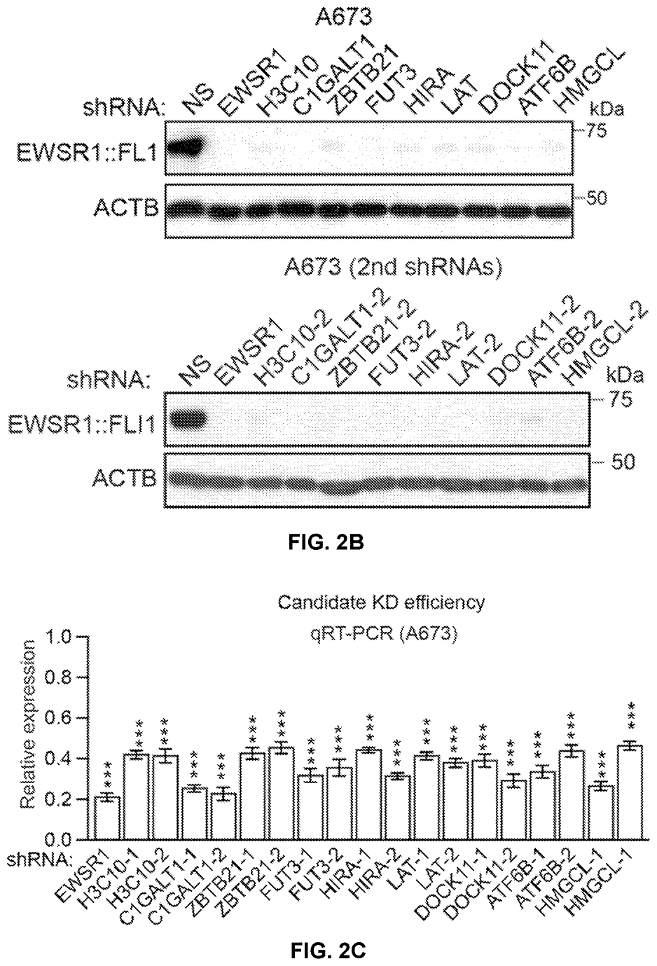

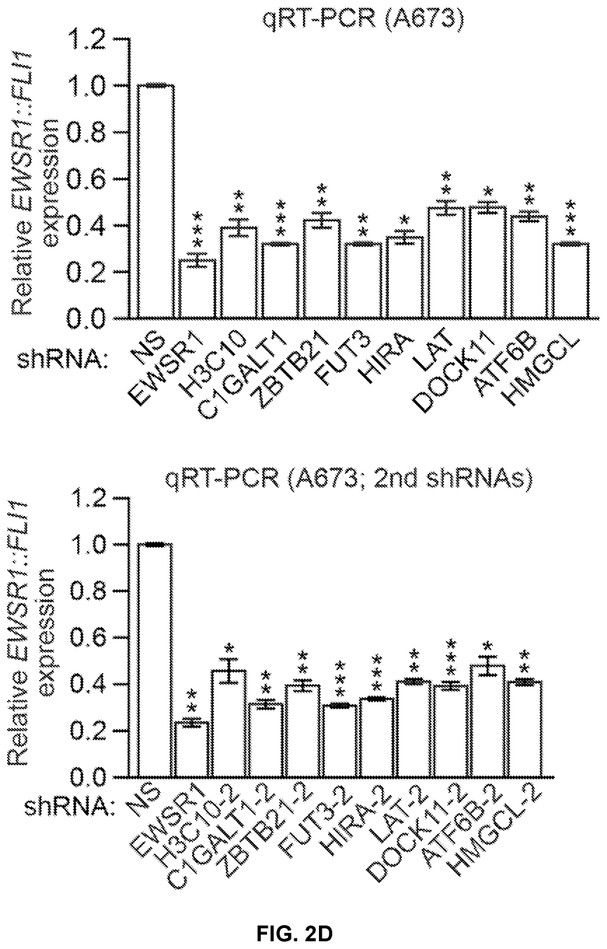

The A673/EWSR1::FLI1^{tdTomato}/Cas9/EGFP reporter cell line was transfected with the Human Brunello CRISPR Knockout Library, which has about 76,000 sgRNAs targeting about 19,000 genes (about 4 sgRNAs per gene). The cells were puromycin selected for 12 days, tdTomato^{low} EGFP^{high} cells were isolated by fluorescence activated cell sorting (FACS) and expanded, and the sgRNAs that were enriched relative to the unsorted population were identified by deep sequencing (FIG. 2A). Approximately 70 genes that had four sgRNAs that were significantly enriched in the tdTomato^{low} EGFP^{high} population were identified. Based on P value, the highest ranking 20 candidates were selected for validation experiments. To validate the candidates, each gene was knocked down in parental A673 cells using two independent shRNAs, and endogenous EWSR1::FLI1 protein levels were monitored by immunoblotting. As a positive control, EWSR1, which encodes the N-terminal portion of the EWSR1::FLI1 fusion gene, was also knocked down. A candidate was considered validated if both shRNAs (1) decreased EWSR1::FLI1 protein levels and (2) decreased mRNA levels of the target gene compared to that obtained with a control non-silencing (NS) shRNA. This approach enabled identification of nine factors that promote EWSR1::FLI1 expression (FIGS. 2B-2C). Quantitative reverse transcription PCR (qRT-PCR) analysis revealed that knockdown of each of these factors also reduced EWSR1::FLI1 mRNA levels (FIG. 2D). In addition, the primary screen identified several other candidates of interest that all had at least two significantly enriched sgRNAs, including: USP14 and USP19, two deubiquitinating enzymes with a known role in EWSR1::FLI1 protein stability (Gierisch et al., 2019; Shukla et al., 2016); HNRNPH1 and SF3B1, two factors previously shown to be required for proper splicing and expression of EWSR1::FLI1 (Grohar et al., 2016); and several components of the Hh signaling pathway (discussed further below) (Table 1).

TABLE 1
Factors that Promote EWSR1::FLI1 Expression.

| | | | | |
|---|---|---|---|---|
| | | | Number of | |
| | | | sgRNAs | |
| Biological | | | identified in | |
| process | Gene symbol | Gene name | the screen | P value* |

Highest-Ranking Candidates

| | | | | |
|---|---|---|---|---|
| Transcriptional | H3C10 | H3 clustered histone 10 | 4 | 2.63E−80 |
| regulation | ZBTB21 | Zinc finger and BTB domain | 4 | 3.96E−63 |
| | | containing 21 | | |
| | HIRA | Histone cell cycle regulator | 4 | 1.50E−54 |
| | ATF6B | Activating transcription factor 6 | 4 | 1.08E−41 |
| | | beta | | |
| Cell signaling | LAT | Linker for activation of T cells | 4 | 3.55E−54 |
| | DOCK11 | Dedicator of cytokinesis 11 | 4 | 1.70E−47 |
| Protein | C1GALT1 | Core 1 synthase, glycoprotein- | 4 | 7.90E−67 |
| metabolism | | N-acetylgalactosamine 3-beta- | | |
| | | galacosyltransferase 1 | | |
| | FUT3 | Fucosyltransferase 3 | 4 | 9.61E−58 |
| | HMGCL | 3-hydroxy-3-methylglutaryl- | 4 | 6.64E−37 |
| | | CoA-lyase | | |

Representative Other Candidates of Interest

| | | | | |
|---|---|---|---|---|
| EWSR1::FLI1 | HNRNPH1 | Heterogeneous nuclear | 2 | 1.74E−82 |
| expression/ | | ribonucleoprotein H1 | | |
| stability | SF3B1 | Splicing factor 3b subunit 1 | 2 | 1.12E−20 |
| | USP14 | Ubiquitin specific peptidase 14 | 4 | 1.40E−07 |
| | USP19 | Ubiquitin specific peptidase 19 | 2 | 4.89E−04 |
| Hh signaling | GLI1 | GLI family zinc finger 1 | 3 | 1.61E−42 |
| | SMO | Smoothened, frizzled class | 2 | 2.55E−10 |
| | | receptor | | |
| C1GALT1 | C1GALT1C1 | C1GALT1 specific chaperone 1 | 3 | 6.22E−44 |
| function | | | | |

*The P value given is for the top-scoring sgRNA of the set.

### Example 3. C1GALT1 is Highly Expressed in ES and is Required for EWSR1::FLI1 Expression and Function

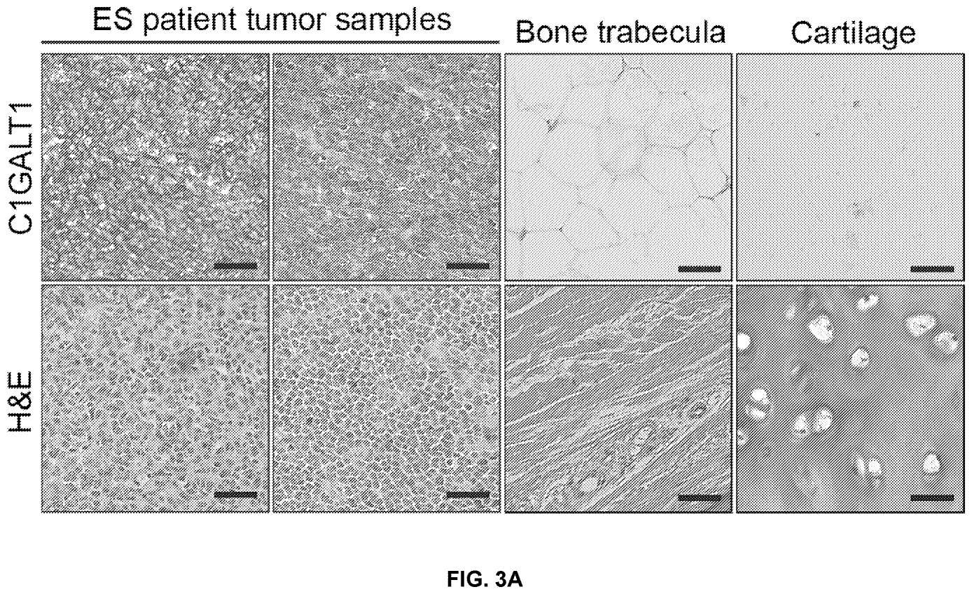

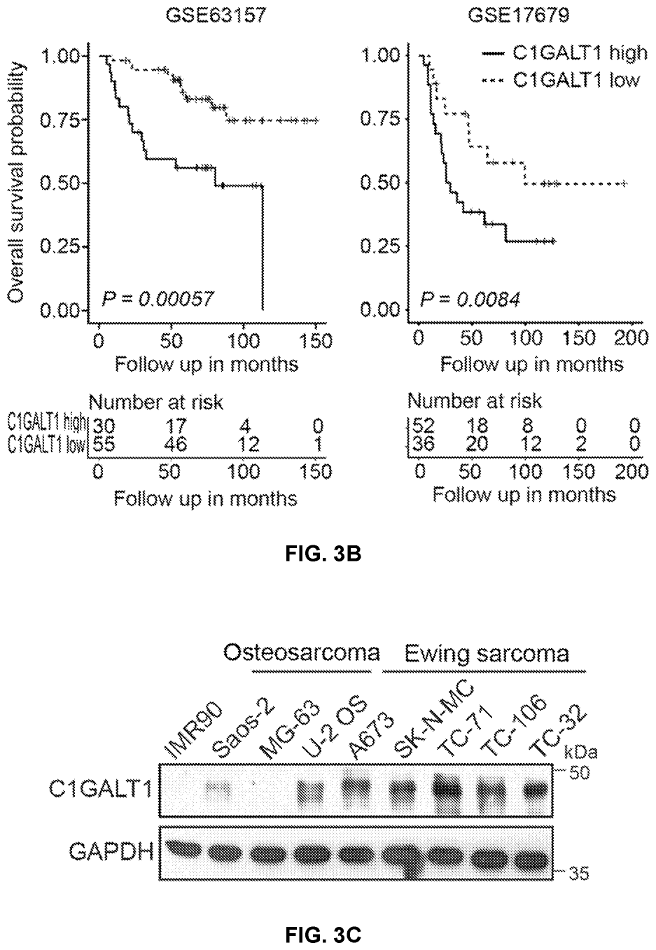

Of the nine validated top-ranking factors, the one of particular interest was core 1 synthase, glycoprotein-N-acetylgalactosamine 3-β-galactosyltransferase 1 (C1GALT1). C1GALT1 plays a critical role in the O—N-acetylgalactosamine-glycosylation (O-GalNAc-glycosylation or O-glycosylation) of proteins by N-acetylgalactosamine-linked (GalNAc- or mucin-type) oligosaccharides. GalNAc-type O-glycans are synthesized in the Golgi by sequential glycosyltransferase reactions (Varki et al., 2022). The initiating event is the addition of a GalNAc monosaccharide to serine or threonine of the target protein to form so-called Tn antigen, following which the emerging glycan structure can diverge into one of eight different core extension pathways (core 1-8). C1GALT1 is the only enzyme that catalyzes the addition of galactose to GalNAc to synthesize the “core 1” structure (also called T antigen), the most common O-glycan core (Varki et al., 2022). Interestingly, Tn antigen is overexpressed in many different cancer types (Fu et al., 2016). C1GALT1 overexpression has been documented in various malignancies, including pancreatic, ovarian, liver, gastric, prostate, esophageal and head and neck cancers, and shown to contribute to the malignant phenotype (e.g., cell proliferation and metastasis) and chemotherapeutic drug sensitivity (Sun et al., 2021). Immunohistochemistry staining revealed high C1GALT1 expression in ES patient tumor samples but not normal bone or cartilage (FIG. 3A). Kaplan-Meier analysis of a published dataset (Savola et al., 2011) revealed that high C1GALT1 expression in Ewing sarcoma is associated with decreased overall survival (FIG. 3B), consistent with observations in other cancers (Wan and Yu, 2021). Finally, consistent with the results in primary patient samples, immunoblot analysis revealed that C1GALT1 is expressed at high levels in human ES cell lines compared to normal human fibroblasts and human osteosarcoma cell lines, another common primary malignant bone tumor in children (FIG. 3C).

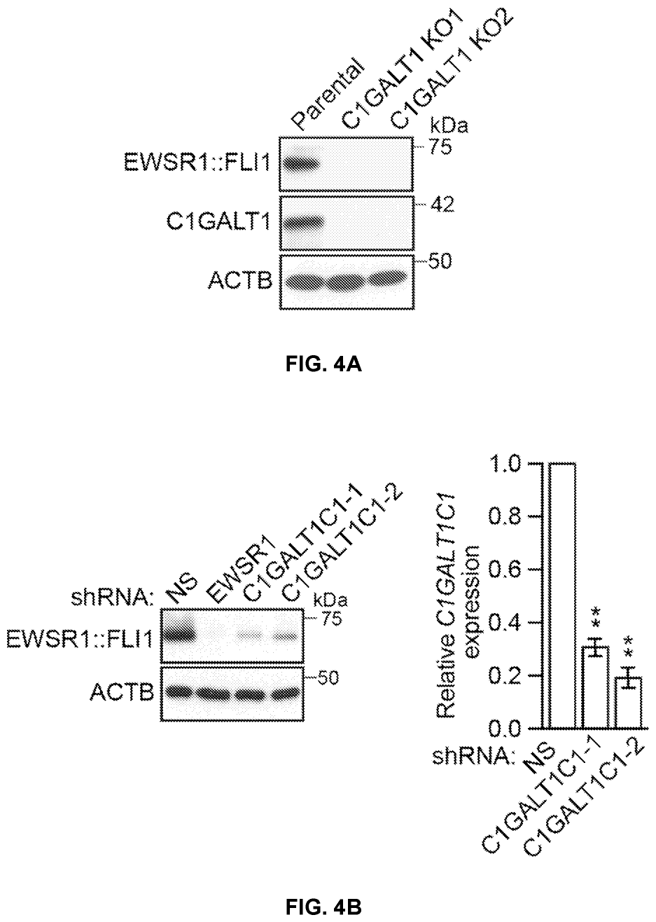

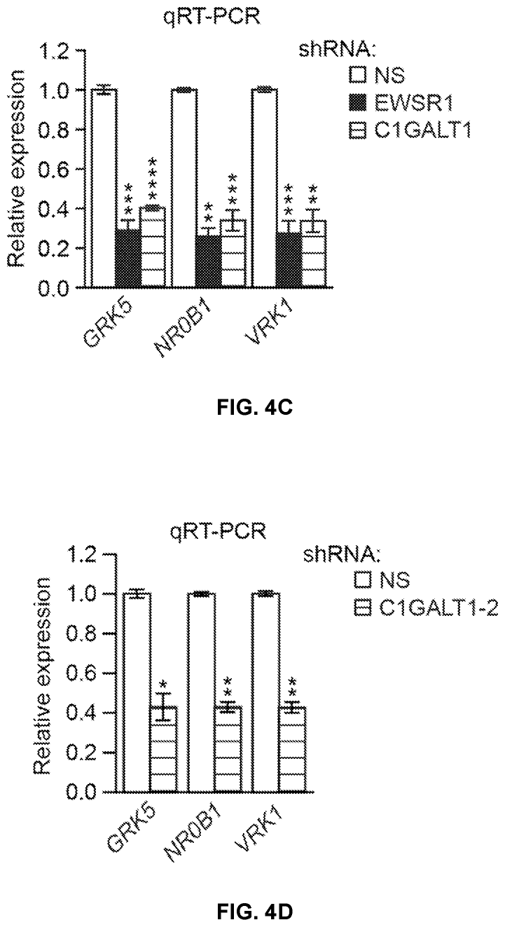

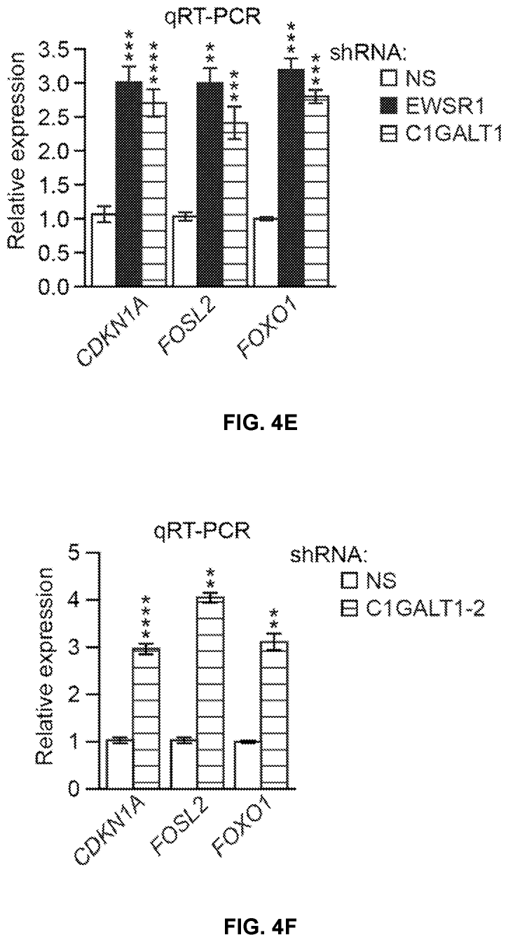

To further validate C1GALT1 as a factor required for EWSR1::FLI1 expression, several additional experiments were performed. FIG. 4A shows that CRISPR/Cas9-mediated knockout of C1GALT1 in A673 cells abolished EWSR1::FLI1 levels. Moreover, shRNA-mediated knockdown of the obligate chaperone of C1GALT1, C1GALT1C1 (also known as COSMC) (Ju and Cummings, 2002), which was also identified in the primary screen (see Table 1), also reduced EWSR1::FLI1 levels (FIG. 4B). Finally, shRNA-mediated knockdown of C1GALT1 reduced expression of genes that are normally positively regulated by EWSR1::FLI1 (FIGS. 4C-4D) and increased expression of genes that are normally negatively regulated by EWSR1::FLI1 (FIGS. 4E-4F). Collectively, the results of these validation experiments demonstrate that biological inhibition of C1GALT1 leads to reduced EWSR1::FLI1 levels and function.

### Example 4. The C1GALT1 Inhibitor Itraconazole Reduces EWSR1::FLI1 Levels and Function

Previous studies have shown that itraconazole (ITZ), an FDA-approved anti-fungal agent that potently inhibits the fungal cytochrome P450 CYP3A4 (Isoherranen et al., 2004), an enzyme required for synthesis of ergosterol (a component of fungal cell membranes), also inhibits C1GALT1 (Lin et al., 2018). Therefore, whether ITZ, like C1GALT1 knockdown, would reduce EWSR1::FLI1 levels and function, was determined. It was found that treatment of parental A673 cells with ITZ led to the dose-dependent reduction of EWSR1::FLI1 mRNA (FIG. 5A) and protein (FIG. 5B) levels. Consistent with previous studies (Kim et al., 2010b), it was found that C1GALT1 protein levels were also reduced in a dose-dependent manner, which is thought to be due to ubiquitin-mediated proteasomal degradation (Kim et al., 2010b). In A673 cells, CYP3A4 expression was not detectable (FIG. 5C), excluding the possibility that ITZ-mediated reduction of EWSR1::FLI1 occurred through inhibition of CYP3A4. Furthermore, another FDA-approved anti-fungal CYP3A4 inhibitor, ketoconazole, which has previously been shown not to inhibit C1GALT1 (Lin et al., 2018), had no effect on EWSR1::FLI1 expression (FIG. 5D). Finally, consistent with the results of FIGS. 4C-4F, treatment of A673 cells with ITZ reduced expression of genes that are normally positively regulated by EWSR1::FLI1 (FIG. 5E) and increased expression of genes that are normally negatively regulated by EWSR1::FLI1 (FIG. 5F). Collectively, these results demonstrate that pharmacological inhibition of C1GALT1 leads to reduced EWSR1::FLI1 levels and function.

### Example 5. EWSR1::FLI1 Expression is Promoted by Hh Signaling

ITZ has been shown to have various anti-cancer activities and, in particular, to inhibit Hh signaling by a mechanism that remains to be determined (Li et al., 2019). In mammalian cells, Hh signal transduction occurs in a specialized organelle called the primary cilia (Bangs and Anderson, 2017). Canonical activation of the Hh pathway occurs when a Hh ligand binds to the transmembrane receptor PTCHI, thereby relieving its inhibition of SMO, a seven-pass transmembrane G-protein coupled receptor that is the key signal transducer of the Hh pathway. Activation of SMO promotes downstream activation and nuclear translocation of key effectors of the Hh pathway, the zinc-finger transcription factors GLI1 and GLI2 (transcriptional activators) and GLI3 (a transcriptional repressor). Translocation of GLI1 and GLI2 to the nucleus results in transcriptional activation of Hh-dependent target genes, which include PTCHI, SMO and GLI1, thereby establishing a series of positive and negative feedback loops.

Aberrant activation of Hh signaling is thought to be involved in up to 25% of cancers including, classically, basal cell carcinoma, medulloblastoma, and rhabdomyosarcoma, and more recently pancreatic, lung, prostate, ovarian, and breast cancers (Skoda et al., 2018). In basal cell carcinoma and a subset of medulloblastomas, aberrant Hh signaling results from a Hh pathway activating mutation such as a PTCHI loss-of-function or a SMO gain-of-function mutation. However, the majority of cancers with aberrant Hh signaling lack pathway activating mutations (Liu et al., 2011). Notably, aberrant Hh signaling has also been found in ES, and is thought to be due to increased expression of Hh pathway components including GLI1, SMO and PTCH (Lezot et al., 2020). In particular, GLI1 is a known transcriptional target of EWSR1::FLI1 (Beauchamp et al., 2009; Joo et al., 2009; Zwerner et al., 2008).

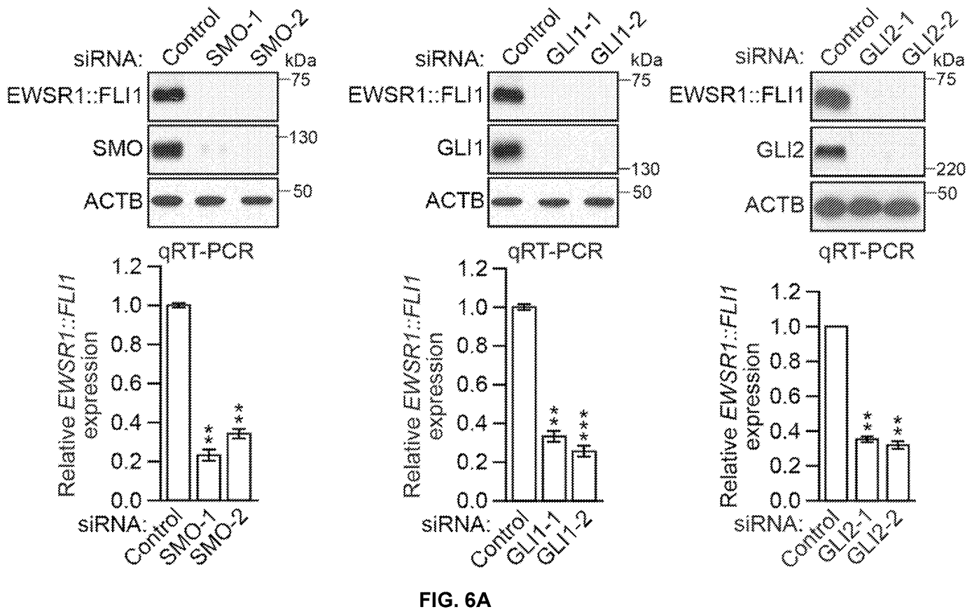

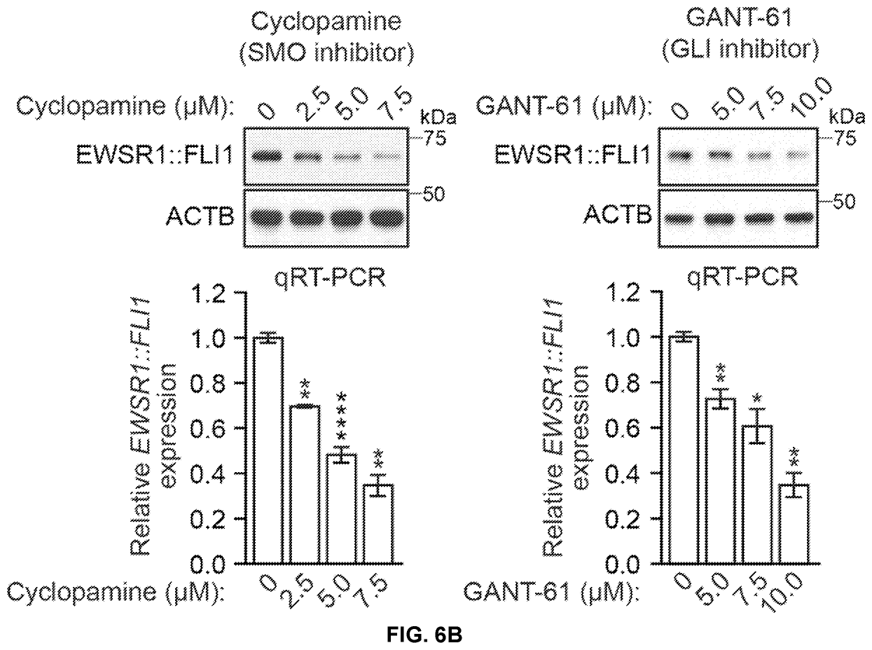

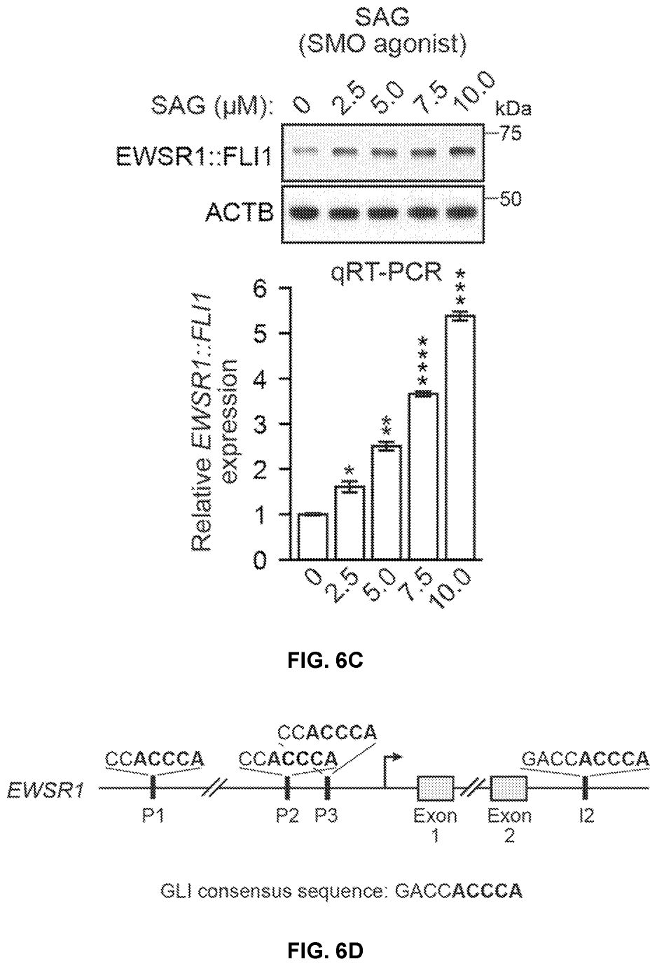

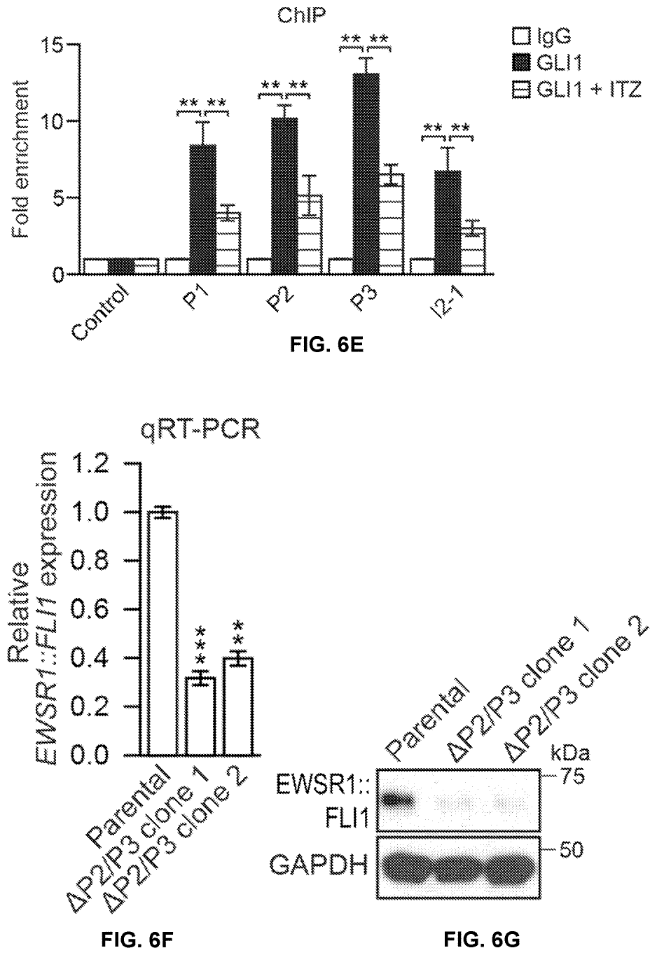

Notably, the CRISPR/Cas9 screen described herein identified multiple Hh signaling components, including SMO and GLI1 (see Table 1). Based upon these considerations, a series of experiments were performed to determine whether Hh signaling promotes expression of EWSR1::FLI1. FIG. 6A shows that knockdown of SMO (left), GLI1 (middle), or GLI2 (right) using two independent short interfering RNAs (siRNAs) resulted in reduced levels of EWSR1::FLI1 at both the mRNA and protein levels. Similarly, treatment of A673 cells with a pharmacological inhibitor of SMO (cyclopamine; (Peukert et al., 2009)) or GLI (GANT-61; (Lauth et al., 2007)) led to the dose-dependent reduction of EWSR1::FLI1 mRNA and protein levels (FIG. 6B). Conversely, treatment of A673 cells with the Hh activator Smoothened Agonist hydrochloride (SAG), a pharmacological agonist of SMO (Chen et al., 2002), led to the dose-dependent increase of EWSR1::FLI1 at both the mRNA and protein levels (FIG. 6C).

As mentioned above, Hh signaling culminates in the activation of the transcription factor GLI1. Therefore, it was asked whether GLI1 was bound to the EWSR1 promoter, which drives expression of EWSR1::FLI1. Previous studies have shown that GLI transcription factors bind with high affinity to the consensus sequence GACCACCCA (Kinzler and Vogelstein, 1990), of which the last five residues are the most critical for binding (Winklmayr et al., 2010). Bioinformatic analysis identified four candidate GLI-binding sites in EWSR1, three upstream of the transcription start-site and one downstream in the second intron (FIG. 6D). The chromatin immunoprecipitation (ChIP) experiment of FIG. 6E shows that GLI1 is bound to all four of the predicted GLI-binding sites in EWSR1, and that GLI1 binding is reduced following inhibition of Hh signaling by ITZ treatment. Deletion of two GLI-binding sites, located in the promoter region immediately upstream of the transcription start site (TSS), by CRISPR/Cas9-mediated gene editing resulted in decreased EWSR1::FLI1 expression at the mRNA (FIG. 6F) and protein (FIG. 6G) levels. Collectively, the results of these experiments indicate that in ES cells, Hh signaling promotes EWSR1::FLI1 expression. Combined with previous findings showing EWSR1::FLI1 regulates Hh signaling (Joo et al., 2009), the results suggest a feedback loop in which Hh signaling promotes EWSR1::FLI1 expression, which in turn increases Hh signaling.

### Example 6. C1GALT1 Stimulates Hh Signaling in ES Cells by Promoting O—N-acetylgalactosamine-glycosylation (O-GalNAc-glycosylation or O-glycosylation) and Stabilization of SMO

The results described above suggest that C1GALT1 promotes EWSR1::FLI1 expression by stimulating Hh signaling. A series of experiments were performed to confirm this pathway and determine its mechanistic basis. In A673 cells, shRNA-mediated knockdown of C1GALT1 (FIG. 7A) or pharmacological inhibition of C1GALT1 with ITZ (FIG. 7B) led to reduced mRNA and protein levels of SMO and GLI1, a common readout of Hh signaling, consistent with previous results showing ITZ inhibits Hh signaling (Li et al., 2019). To independently confirm this finding, the effects of C1GALT1 knockdown in a well-characterized reporter assay for monitoring the activity of the Hh signaling pathway, in which luciferase expression is driven by multiple GLI-binding sites (Sasaki et al., 1997), were assessed. It was found that knockdown of C1GALT1, or as a control SMO, reduced GLI-driven luciferase expression in A673 cells (FIG. 7C).

Conversely, whether ectopic expression of C1GALT1 could promote Hh signaling was examined. These experiments were performed in NIH 3T3 cells, which have a low basal level of Hh signaling and are a commonly used experimental model for studying Hh signaling regulation (Taipale et al., 2000). Similar to treatment with the Hh activator SAG, ectopic expression of C1GALT1 (FIG. 7D) resulted in increased levels of SMO and GLI1 (FIG. 7E), indicative of increased Hh signaling. Ectopic expression of C1GALT1 also increased GLI-driven luciferase expression in NIH 3T3 cells, similar to ectopic expression of SMO (FIG. 7F)

SMO is subjected to multiple post-translational modifications, including phosphorylation, ubiquitination, sumoylation and N-glycosylation (Zhang et al., 2021). Previous large-scale glycoproteomic studies identified SMO as a GalNAc-type O-glycan with a core 1 structure (Yang et al., 2018; Yang et al., 2020), suggesting it is a C1GALT1 target. Therefore, the possibility that C1GALT1 stimulates Hh signaling by O-GalNAc-glycosylating SMO was considered. To determine whether SMO harbors the T antigen moiety (produced by C1GALT1) in ES cells, a lectin pull-down assay was performed. In brief, A673 cell protein extracts were incubated with peanut agglutinin (PNA) lectin beads, which bind strongly to the serine/threonine O-linked disaccharide galactosyl (β-1,3)N-acetylgalactosamine (T antigen) (Pereira et al., 1976), and the bound T antigen-containing proteins were analyzed for the presence of SMO by immunoblotting. FIG. 7G shows that SMO bound to PNA lectin beads and that binding was substantially reduced following treatment with O-glycosidase, which removes core 1 and core 3 O-glycans (Varki et al., 2022) (see schematic in FIG. 7G). Furthermore, O-glycosidase treatment resulted in a decrease in the apparent molecular weight of SMO, indicative of removal of O-glycans. Collectively, these findings indicate that SMO is O-glycosylated in ES cells and harbors, at a minimum, Gal-GalNAc dissaccharides (T antigen). Consistent with the idea that SMO is a C1GALT1 target, the co-immunoprecipitation experiment of FIG. 7H shows that SMO and C1GALT1 interact.

Next, whether O-GalNAc-glycosylation of SMO was dependent on C1GALT1 was tested. Because total SMO levels were substantially reduced in C1GALT1 knockdown cells (see FIG. 7A), these experiments were performed by inhibiting C1GALT1 with ITZ, rather than by shRNA knockdown, which enabled monitoring of early time points. The PNA lectin pulldown assay was repeated in cells treated with ITZ, and SMO glycosylation was monitored over time. In parallel, the total levels of SMO in whole cell extracts were also monitored. FIG. 7I shows that treatment of A673 cells with ITZ led to the rapid decrease of O-glycosylated SMO (within 12 hours) with a subsequent reduction of total SMO levels at later time points.

The above results suggest that loss of C1GALT1-mediated O-GalNAc-glycosylation of SMO reduces the stability of SMO. Consistent with this possibility, the cycloheximide chase experiment of FIG. 7J demonstrated that the half-life of SMO was substantially reduced in the presence of ITZ. The reduction in SMO levels observed upon ITZ treatment was reversed by addition of the proteasome inhibitor MG132 or the lysosome inhibitor bafilomycin A1 (FIG. 7K), suggesting that C1GALT1 stabilizes SMO from proteasomal and lysosomal degradation. Next, the hypothesis, that SMO mutants that are unable to be O-glycosylated have reduced stability, was tested. Previous proteomics studies (Yang et al., 2018; Yang et al., 2020) show that SMO residues Thr-55 and Thr-500 are O-glycosylated. SMO Thr-55 and Thr-500 mutants were generated, and the effects on SMO stability were tested. Site-directed mutagenesis was performed to generate SMO derivatives in which Thr-55 or Thr-500 was mutated to Ala, and the mutants were stably expressed as FLAG-tagged versions, in parallel with wild-type SMO, in A673 cells. The results of FIG. 7L show that the SMO (T55A) and SMO (T500A) mutant proteins were not detectable, and that their expression could be restored by addition of MG132 or bafilomycin A1, suggesting the mutants had reduced stability due, in part, to ubiquitin-mediated protein degradation. Consistent with this idea, ubiquitination of SMO (T55A) and SMO (T500A) was detected when protein degradation was blocked by treatment with either MG132 or bafilomycin A1 (FIG. 7M). Previous studies have shown that in the absence of ligand-induced pathway activation, SMO is ubiquitinated and removed from the primary cilia (Desai et al., 2020). Activation of Hh signaling decreases SMO ubiquitination, resulting in its accumulation in the primary cilia. Blocking ubiquitination of SMO, by an E1 ligase inhibitor or by mutating two lysine residues required for ubiquitination, causes SMO to aberrantly accumulate in cilia without pathway activation (Desai et al., 2020). These results suggest that C1GALT1-mediated O-GalNAc-glycosylation of SMO blocks its ubiquitination, causing SMO to aberrantly accumulate in cilia in the absence of ligand-induced pathway activation.

### Example 7. C1GALT1 is Required for EWSR1::FLI1-Mediated Cellular Transformation and is Sufficient to Transform NIH 3T3 Cells

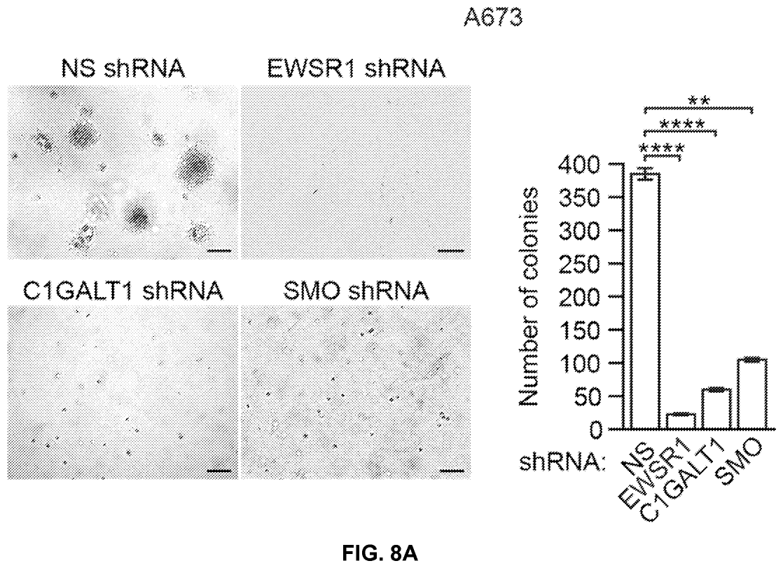

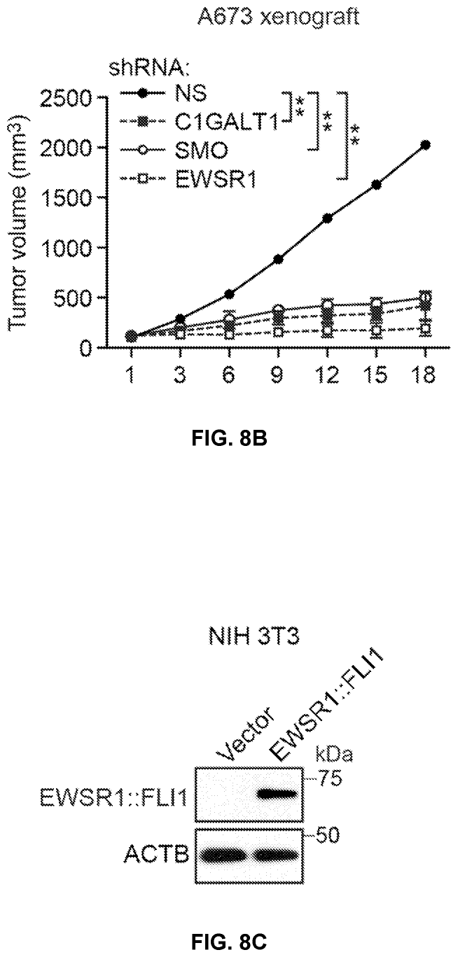

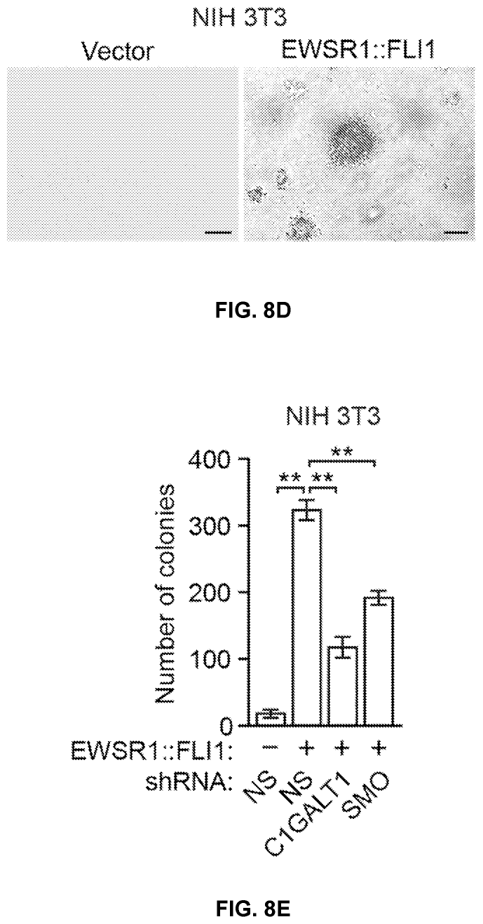

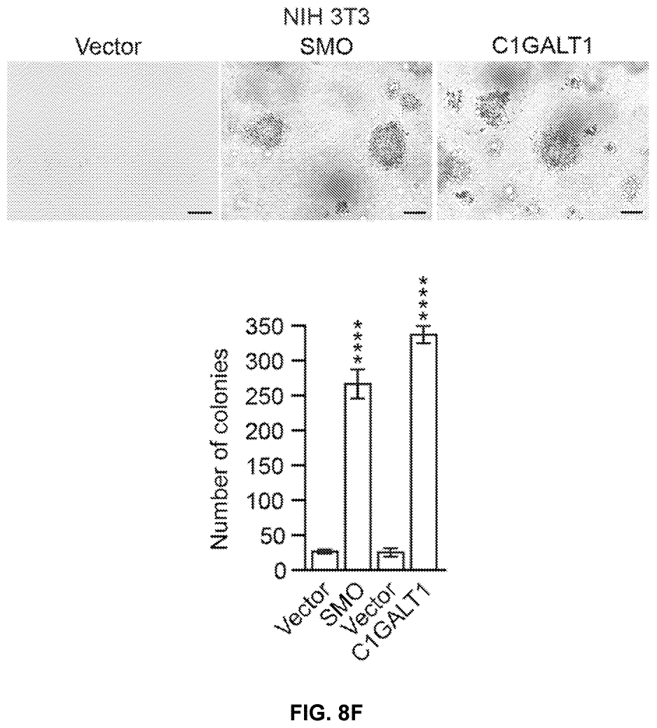

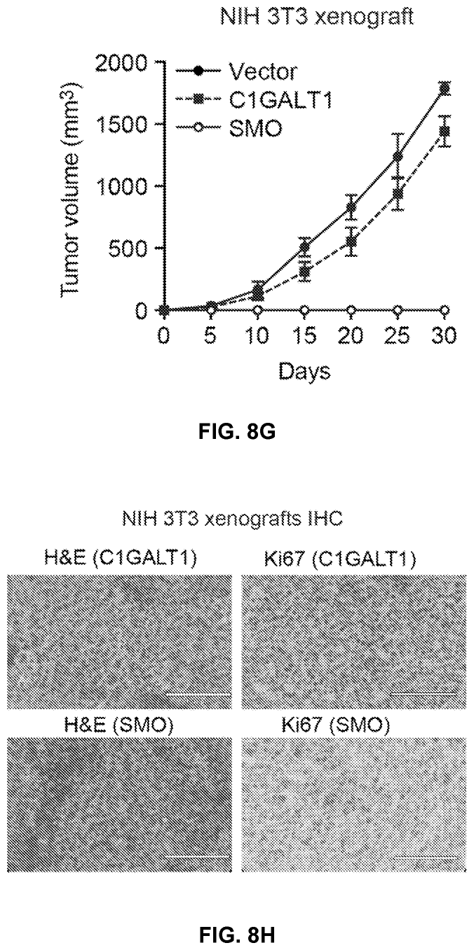

A series of experiments were performed to investigate the role of C1GALT1 in cellular transformation. As mentioned above, A673 ES cells do not require EWSR1::FLI1 for proliferation in cell culture but are dependent on EWSR1::FLI1 for the transformed phenotype (Smith et al., 2006). shRNA-mediated knockdown of EWSR1 substantially reduced the ability of A673 cells to form colonies in soft agar, a characteristic property of transformed cells (FIG. 8A), and to form tumors following subcutaneous injection in mice (FIG. 8B). A similar reduction in colony formation and tumor growth was observed following knockdown of SMO or, notably, C1GALT1 (FIGS. 8A-8B). Thus, Hh signaling and C1GALT1 are required for the transformed phenotype of A673 cells. To independently confirm these results, whether Hh signaling and C1GALT1 are required for the ability of EWSR1::FLI1 to transform NIH 3T3 cells was examined. Consistent with previous studies (May et al., 1993), ectopic expression of EWSR1::FLI1 (FIG. 8C) enabled NIH 3T3 cells to form colonies in soft agar (FIGS. 8D-8E), which was substantially reduced by shRNA knockdown of SMO, as expected, or C1GALT1 (FIG. 8E). Collectively, the results of FIGS. 8A-8E indicate that C1GALT1 and Hh signaling are required for EWSR1::FLI1-mediated cellular transformation.

The converse experiment was performed to ask whether C1GALT1 was sufficient for cellular transformation. Previous studies have shown that NIH 3T3 cells can be transformed by increased Hh signaling (Lauth et al., 2007; Pusapati et al., 2018a; Pusapati et al., 2018b). Therefore, whether ectopic expression of C1GALT1, which increases Hh signaling (FIG. 7E), could also transform NIH 3T3 cells, was examined. Ectopic expression of either C1GALT1 or, as a positive control, SMO enabled NIH 3T3 cells to form colonies in soft agar (FIG. 8F) and tumors following subcutaneous injection in mice (FIG. 8G). As evidenced by positive K167 staining (FIG. 8H), tumors formed from NIH 3T3 cells ectopically expressing C1GALT1 or SMO were actively proliferating. Based on these results, and on those of FIGS. 7E-7F, it was concluded that C1GALT1 transforms NIH 3T3 cells by stimulating Hh signaling.

### Example 8. ITZ Inhibits Proliferation of Human ES Cell Lines in Culture and Suppresses Growth of ES Xenografts in Mice

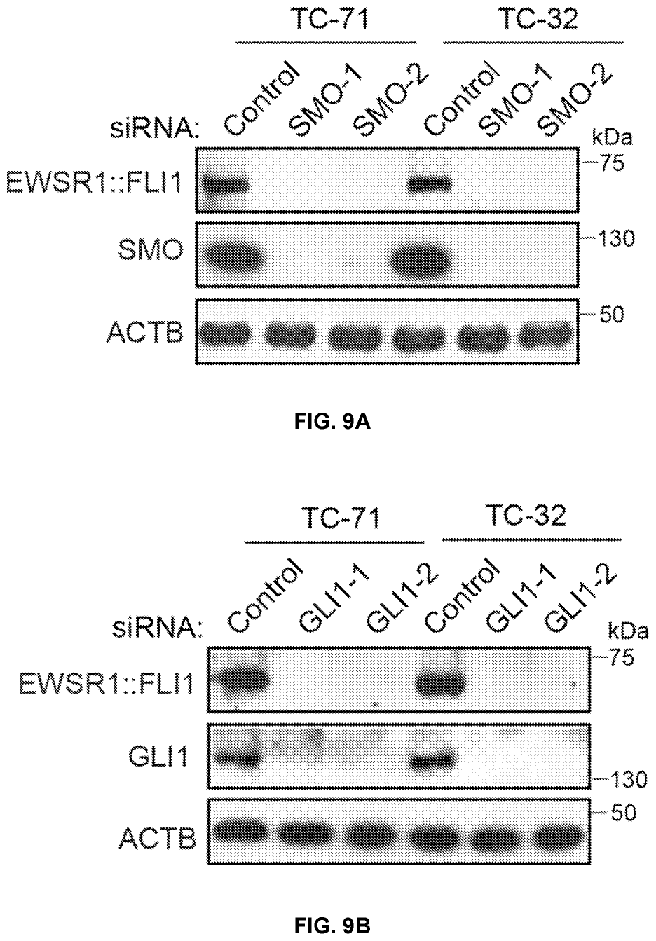

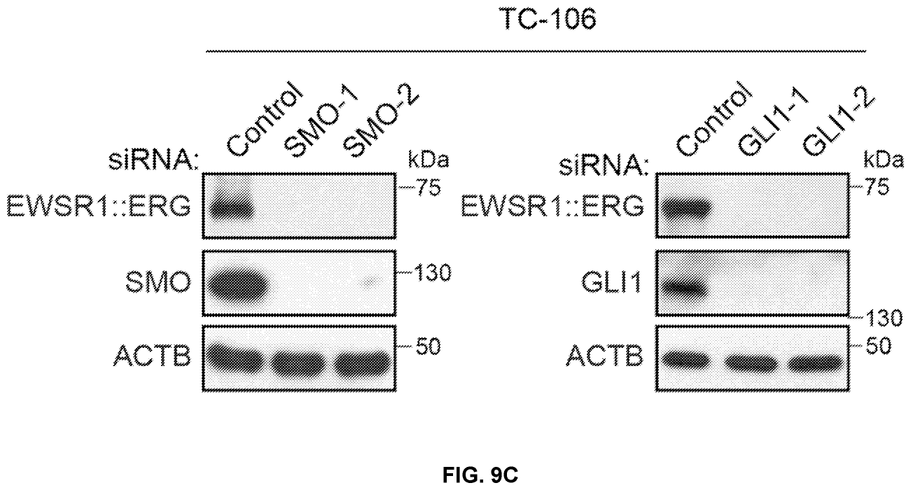

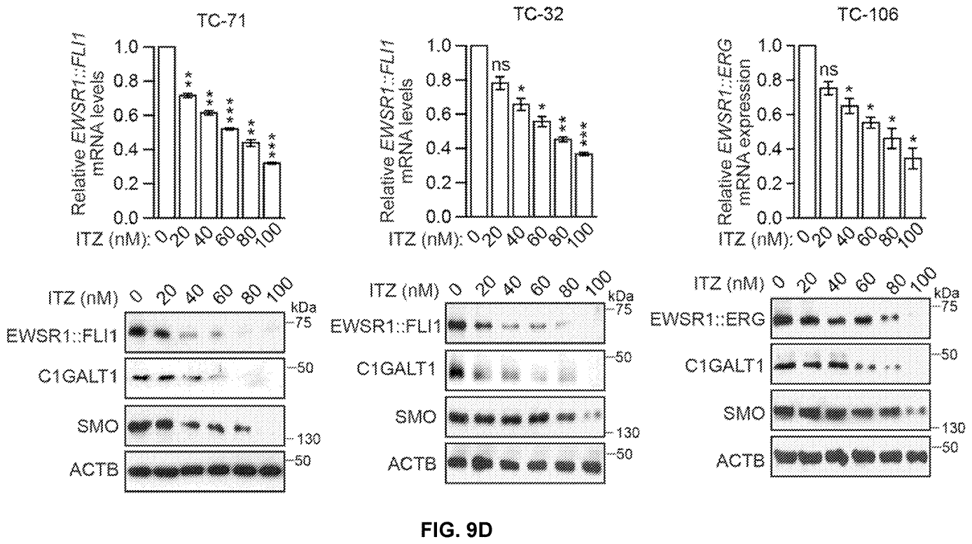

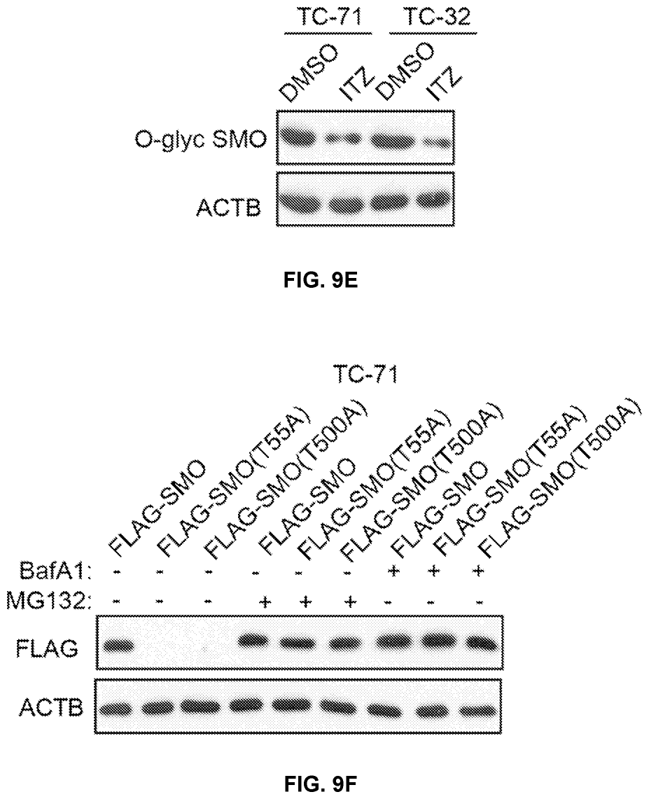

Next, the effect of ITZ on the proliferation of ES cells in culture and growth of ES xenografts in mice was analyzed. These experiments were performed in a panel of ES cell lines that express high levels of C1GALT1 (see FIG. 3C): TC-32 and TC-71, which require EWSR1::FLI1 for viability and proliferation in culture (Erkizan et al., 2011), and TC-106, whose proliferation and viability in culture is dependent or another ES fusion gene, EWSR1::ERG (Erkizan et al., 2011), which shares the EWSR1 promoter and is therefore expected to be transcriptionally regulated by the same mechanism as EWSR1::FLI1. Several key experiments were performed to demonstrate in these ES cell lines that EWSR1::FLI1/ERG expression is promoted by the same C1GALT1-mediated Hh signaling mechanism that was elucidated in A673 cells. Consistent with the results in A673 cells, Hh signaling was required for expression of EWSR1::FLI1/ERG (FIG. 9A-C), and ITZ treatment reduced both Hh signaling and EWSR1::FLI1/ERG expression (FIG. 9D). Moreover, in TC-71 cells, ITZ treatment reduced SMO O-GalNAc-glycosylation (FIG. 9E), and O-GalNAc-glycosylation-defective SMO mutants had reduced stability that could be restored by treatment with either MG132 or BafA1 (FIG. 9F).

Treatment of TC-71 cells with ITZ led to a dose-dependent reduction in cell viability (FIG. 10A), which was due, at least in part, to induction of apoptosis, as evidenced by an increase in poly (ADP-ribose) polymerase 1 (PARP1) cleavage (FIG. 10B). Similar results were observed in TC-32 cells (FIG. 10C-D). In TC-71 cells, ITZ displayed substantially higher efficacy compared to the FDA-approved SMO antagonists sonidegib and vismodegib (Rimkus et al., 2016), which required a much higher concentration, 50-200 times that of ITZ, to achieve a comparable decrease in cell viability (compare FIGS. 10A and 10E). Notably, ectopic expression of EWSR1::FLI1 under the control of a constitutive CMV promoter counteracted the decreased proliferation following ITZ treatment in TC-71 cells, indicating that the reduced levels of EWSR1::FLI1 following ITZ treatment are responsible for the proliferation defect (FIG. 10F).

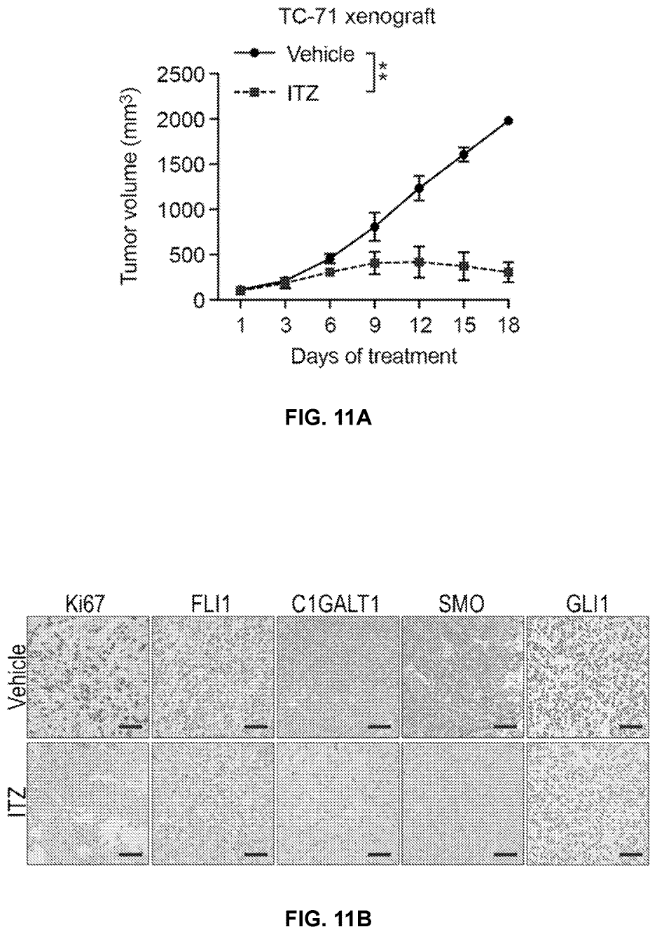

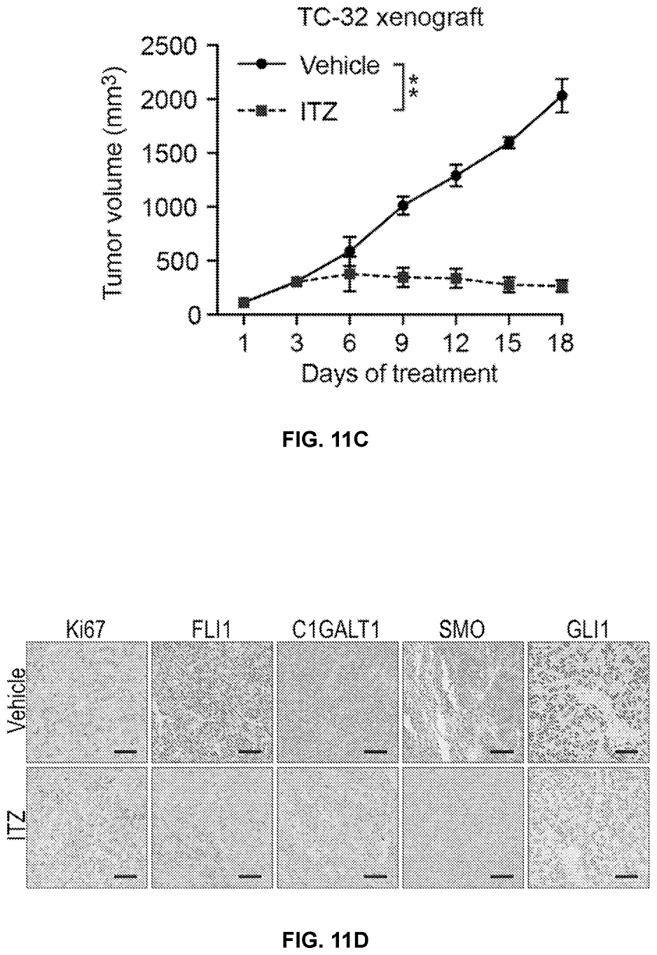

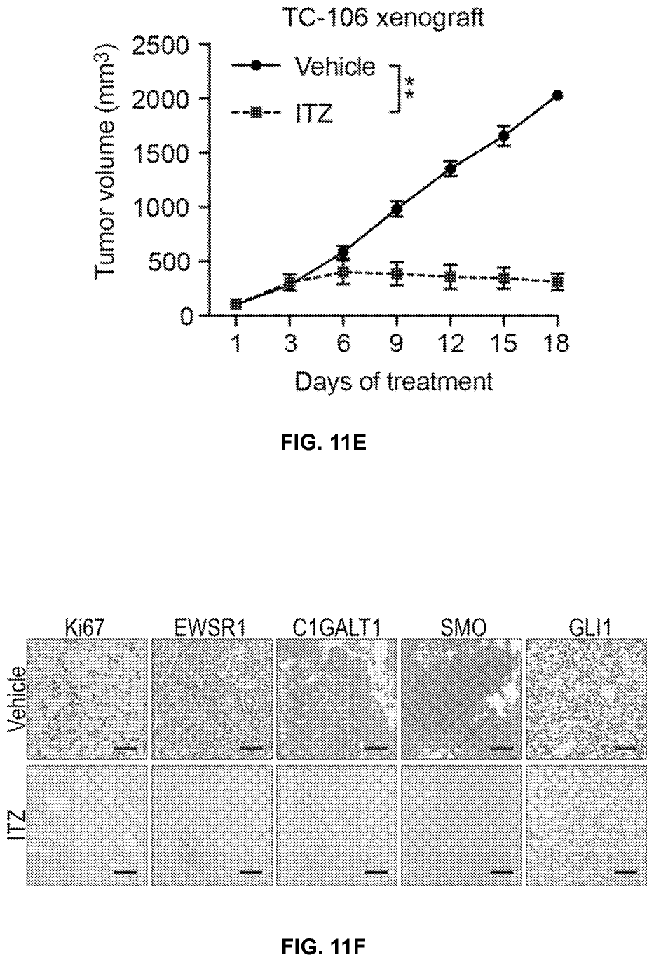

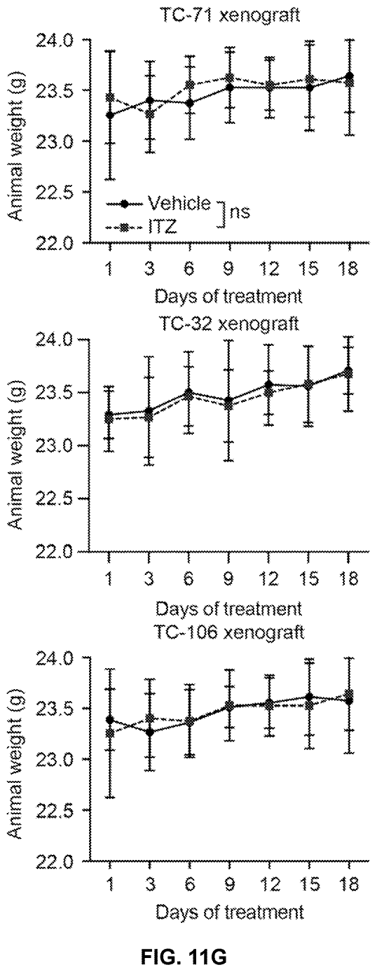

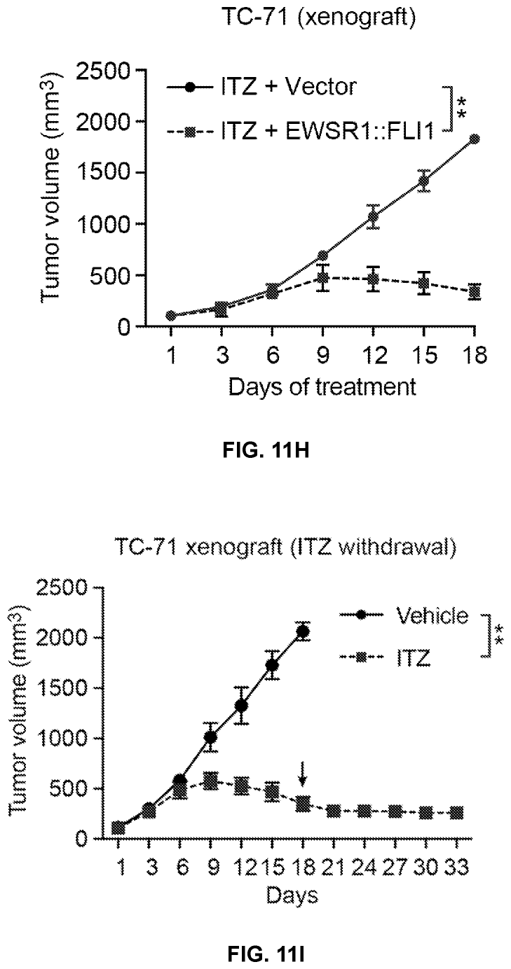

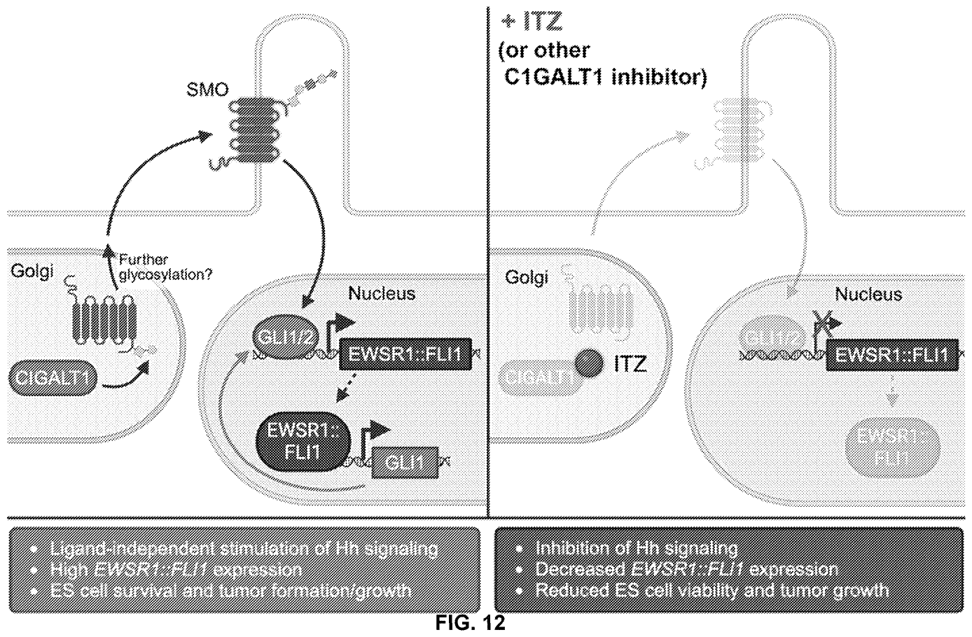

The effect of ITZ treatment on growth of subcutaneous ES xenografts in mice was also tested. In brief, ES cells were injected subcutaneously into the flank of NSG mice, and once tumors were established, mice were treated daily with 100 mg/kg ITZ by oral gavage. FIG. 11A shows that the growth of xenografts derived from TC-71 cells was markedly suppressed by ITZ treatment. Immunohistochemical analysis of tumors revealed substantially reduced levels of FLI1, C1GALT1, SMO and GLI1, as well as reduced Ki67 staining, EWSR1::FLI1 in TC-71 cell tumors in ITZ-treated mice (FIG. 11B), demonstrating that the reduction in EWSR1::FLI1 expression following ITZ treatment observed in cultured cells (see FIGS. 5A-5B) also occurs in vivo. Similar results were observed for TC-32 (FIG. 11C-11D) and TC-106 (FIG. 11E-11F) cells. The dose of ITZ used in these experiments (100 mg/kg) is non-toxic and well tolerated, as evidenced by the absence of weight loss in ITZ-treated mice (FIG. 11G). The ability of ITZ to suppress TC-71 xenograft growth was counteracted by ectopic expression of EWSR1::FLI1 (FIG. 11H), indicating that the reduced growth of ES tumors following ITZ treatment is due to decreased EWSR1::FLI1 levels. To test whether there is tumor recurrence following ITZ withdrawal, mice with established TC-71 xenograft tumors were treated with ITZ for 18 days, and then drug treatment was stopped and tumor growth was monitored for an additional 15 days. FIG. 11I shows that growth of TC-71 xenografts did not increase following removal of the drug, suggesting there was no tumor recurrence following ITZ withdrawal.

Based upon the combined results, a model for the relationship between C1GALT1, Hh signaling and EWSR1::FLI1 expression is presented in FIG. 12. In Ewing sarcoma, high C1GALT1 expression results in non-canonical activation of the Hh signaling pathway through O-GalNAc-glycosylation and stabilization of the pivotal Hh signaling component SMO. As a result, SMO stimulates Hh signaling, which in turn promotes EWS-FLI expression. Notably, whereas previous studies have found that EWSR1::FLI1 promotes GLI1 expression and thus Hh signaling (Beauchamp et al., 2009; Joo et al., 2009; Zwerner et al., 2008), these studies show that Hh signaling also promotes EWSR1::FLI1 expression, thereby establishing a positive feedback loop. Inhibition of C1GALT1 using ITZ is found to reduce EWSR1::FLI1 levels in ES cell lines resulting in cell death and suppress growth of tumors derived from ES cell lines in conventional subcutaneous xenograft mouse models.

### Example 9. Biological Inhibition of C1GALT1 Reduces Hh Signaling in Ovarian and Pancreatic Cancers

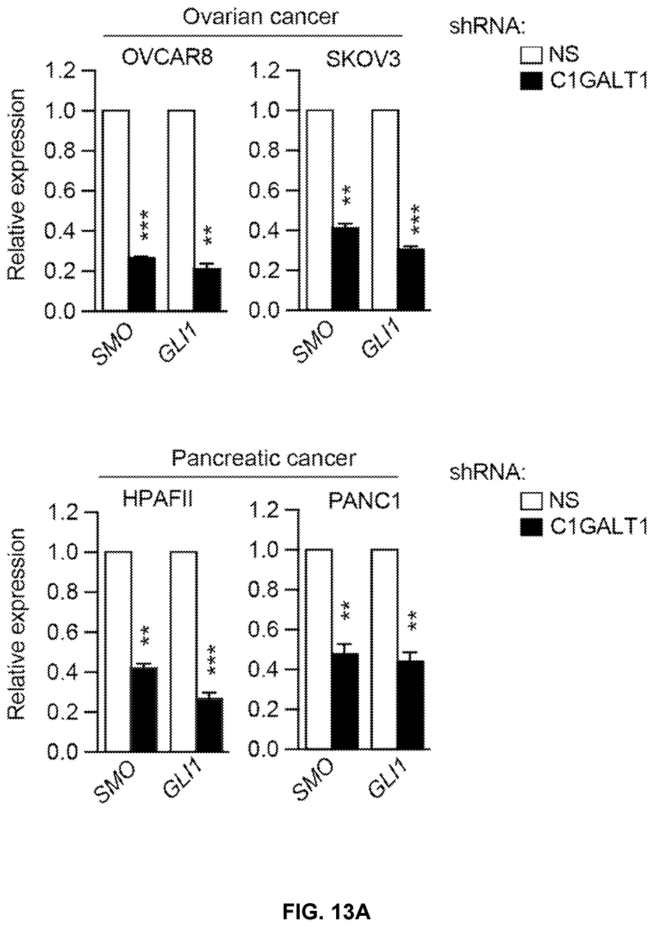

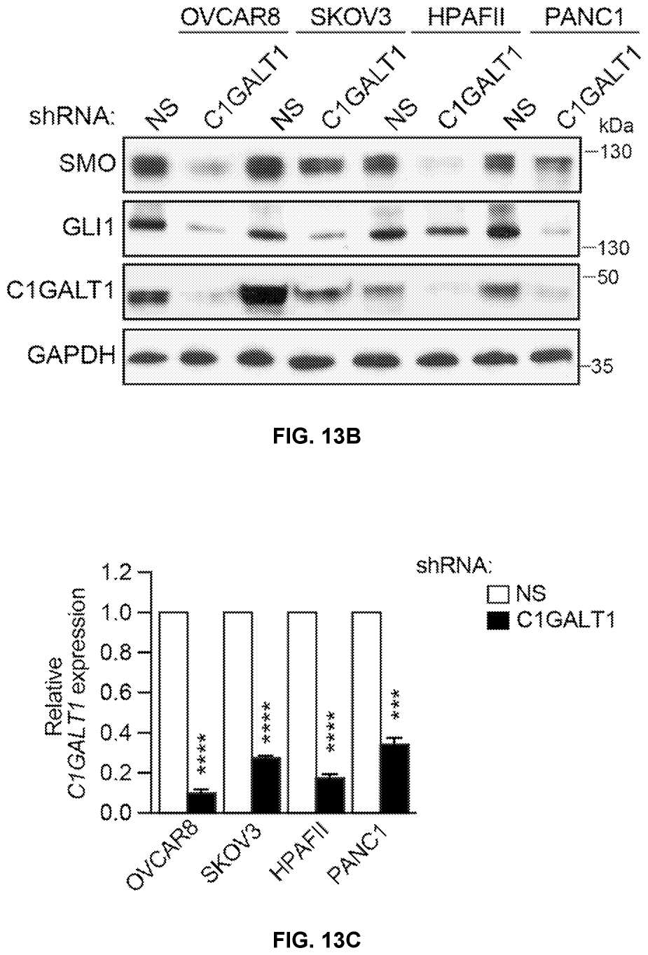

As mentioned above, C1GALT1 is highly expressed in other cancer types, including ovarian (Chou et al., 2017) and pancreatic cancers (Kuo et al., 2021). It was therefore determined whether C1GALT1 promotes Hh signaling in ovarian and pancreatic cancers. FIGS. 13A-C. 12 shows that shRNA-mediated knockdown of C1GALT1 in human ovarian cancer cell lines (OVCAR-8 and SK-OV-3) or pancreatic ductal adenocarcinoma cell lines (Pa03C and PANC-1) led to reduced expression of SMO and GLI1 at the mRNA and protein levels, indicative of reduced Hh pathway activity. These results suggest that the mechanism that current studies identified in ES (i.e., that C1GALT1 promotes Hh signaling) also occurs in other cancers with high C1GALT1 expression. Thus, C1GALT1 may be a therapeutic target in other cancers associated with high C1GALT1 expression and aberrant Hh signaling.

The results of Examples 2-9 show, for the first time, that C1GALT1 promotes Hh signaling, and reveal C1GALT1 as a novel target for inhibiting Hh signaling. The link between C1GALT1 and Hh signaling had not been previously described. These results strongly suggest that C1GALT1 is a therapeutic target in ES and, because ITZ is FDA approved, the results suggest ITZ can be rapidly advanced into ES clinical trials. Furthermore, C1GALT1 may be a therapeutic target for other cancers hallmarked by C1GALT1 overexpression and aberrant Hh signaling, such as ovarian, pancreatic, liver, gastric, prostate, ovarian, esophageal, and head and neck cancers.

Unlike ITZ, treatment of ES cells with another FDA-approved anti-fungal drug, ketoconazole, did not result in reduced EWSR1::FLI1 expression. Previous studies have shown that, unlike ITZ, ketoconazole does not inhibit C1GALT1 activity (Lin et al., 2018). Thus, not all anti-fungal triazole compounds than exhibit anti-cancer activities are expected to inhibit C1GALT1 and Hh signaling. Notably, ITZ analogues with greater potency and Hh targeting activity have been developed (Li et al., 2018; Li et al., 2020; Pace et al., 2019; Shi et al., 2011; Wen et al., 2020).

Current FDA-approved Hh pathway inhibitors, vismodegib (also called Erivedge) and sonidegib (Odomzo), are SMO antagonists that act by directly targeting SMO (Nguyen and Cho, 2022). The Examples demonstrate a new modality for targeting SMO activity: inhibition of C1GALT1. The C1GALT1 inhibitor ITZ acts on SMO through a mechanism that is distinct from other SMO antagonists (Kim et al., 2010b). Furthermore, ITZ in combination with arsenic trioxide, which has been shown to antagonize Hh signaling by targeting GLI (Kim et al., 2010a) display inhibitory activity toward SMO mutants that confer resistance to other SMO antagonists (Kim et al., 2013). Collectively, these results suggest that inhibition of C1GALT1 may be more efficacious than currently available SMO antagonists.

TABLE 2
List of primer sequences used for cloning,
RT-PCR, qRT-PCR and ChIP

| SEQ ID | | | |
|---|---|---|---|
| NO. | | | |
| Cloning | Primer Name | Direction | Primer Sequence (5′ → 3′) |
| 1 | FLI1_C-term | Forward | CGACGGCGCGCCGCAATTCCTCCTGG |
| 2 | FLI1_C-term | Reverse | CATCGCTAGCGTACGTGGGTGTTAGG |
| 3 | FLI1_3′UTR | Forward | CGACCTCGAGATTTTCACCCAACTGG |
| 4 | FLI1_3′UTR | Reverse | CATCGCGGCCGCAAGCGATTGATTCG |

RT-PCR

| | | | |
|---|---|---|---|
| 5 | FLI1_Ex9 | Forward | GTGACAAATAGAGGTGGGAAGC |
| 6 | tdTomato | Reverse | CACCTTGAAGCGCATGAACT |

qRT-PCR

| | | | |
|---|---|---|---|
| 7 | ATF6B | Forward | GAAGCTTATGGCAGAGATGCAC |
| 8 | ATF6B | Reverse | CAGTGCTTTCCAAATAGATGGG |
| 9 | C1GALT1 | Forward | TCATGCAAGGCATTCAGATG |
| 10 | C1GALT1 | Reverse | ATGGGTTCTTCAGGGTCGTA |
| 11 | C1GALT1C1 | Forward | ATGAGCATCATCACCTACAAGC |
| 12 | C1GALT1C1 | Reverse | CAGCCCAAAGACTCACATCTTT |
| 13 | CDKN1A | Forward | AGGTGGACCTGGAGACTCTCAG |
| 14 | CDKN1A | Reverse | TCCTCTTGGAGAAGATCAGCCG |
| 15 | DOCK11 | Forward | CTGCCCAAATGAAGGAACAT |
| 16 | DOCK11 | Reverse | CACATTGCAGCCTCAGAAAA |
| 17 | EWSR1 | Forward | ATGGGTCGCATGGGAGGC |
| 18 | EWSR1 | Reverse | GCCTTACACTGGTTGCACTCTGT |
| 19 | EWSR1::FLI1 | Forward | AGTTACCCACCCCAAACTGGATC |
| 20 | EWSR1::FLI1 | Reverse | GCAGCTCCAGGAGGAATTGC |
| 87 | EWSR1::ERG | Forward | TCCAAGTCAATATAGCCAACAGAG |
| 88 | EWSR1::ERG | Reverse | CTGTGGAAGGAGATGGTTGAG |
| 21 | FOSL2 | Forward | CGGACAATCGTCTGAGCG |
| 22 | FOSL2 | Reverse | CCGTATGATGGGGGGC |
| 23 | FOX01 | Forward | TGGTCAAGAGCGTGCCCTACT |
| 24 | FOX01 | Reverse | CCCGCTCTTGCCACCCTCTG |
| 25 | FUT3 | Forward | GCTGGAAACTGCAGCAGGAATC |
| 26 | FUT3 | Reverse | TCAGGTGAACCAAGCCGCT |
| 27 | GAPDH | Forward | TGCACCACCAACTGCTTAGC |
| 28 | GAPDH | Reverse | GGCATGGACTGTGGTCATGAG |
| 29 | GLI1 | Forward | CAGCTACATCAACTCCGGCCAATAGGG |
| 30 | GLI1 | Reverse | TGCTGCGGCGTTCAAGAGAGACTG |
| 31 | GRK5 | Forward | GACCACACAGACGACTTC |
| 32 | GRK5 | Reverse | CGTTCAGCTCCTTAAAGCATTC |
| 33 | H3C10 | Forward | TTCAGCGTCTGGTGCGTGAGAT |
| 34 | H3C10 | Reverse | CAGGTTGGTGTCCTCAAAGAGC |
| 35 | HIRA | Forward | CGGTGGTCAAACAGTGGGAT |
| 36 | HIRA | Reverse | ATGGAGACACACCGCCATTG |
| 37 | HMGCL | Forward | TCTGGGCCCAAGATGGCAGCAATG |
| 38 | HMGCL | Reverse | CTGAGGCAGCTCCAAGATGACT |
| 39 | LAT | Forward | CAGACCTGCTCCCCATCCCA |
| 40 | LAT | Reverse | TCAGGAAGCACCACCAGGTAGCCTG |
| 41 | NROB1 | Forward | CTTCTGTACCGCTGCTGCTT |
| 42 | NROB1 | Reverse | GCAGACCACCTGTGGACTCTT |
| 43 | SMO | Forward | GGCTGCTGAGTGAGAAG |
| 44 | SMO | Reverse | CTGGTTGAAGAAGTCGTAGAAG |
| 45 | VRK1 | Forward | CCAACGAGCTGCAAAACC |
| 46 | VRK1 | Reverse | TGTCATGTAGACCAGACCCCC |
| 47 | ZBTB21 | Forward | TGCCCTGAATGAGGAGCG |
| 48 | ZBTB21 | Reverse | TAAACTCTGAAAGTATTCGCTGCTG |

ChIP

| | | | |
|---|---|---|---|
| 49 | P1 | Forward | GTGTGCACGGCTCCTTGGTAT |
| 50 | P1 | Reverse | CCTGGCCTGTATCCTTTGCCTG |
| 51 | P2 | Forward | CTCCACCTCGACCGTTACCG |
| 52 | P2 | Reverse | GCGCGCTCTGCAGTGAGTAC |
| 53 | P3 | Forward | CTGAATCCATTCCGCGCACAC |
| 54 | P3 | Reverse | GGACCCGGACTAGAACAACTGC |
| 55 | I2 | Forward | GCAGCTACAGTGCTTACACCG |
| 56 | I2 | Reverse | TCCTTCGCACTCCCACTGT |
| 89 | Control Locus | Forward | CATCCCTGGACTGATTGTCA |
| 90 | Control Locus | Reverse | GGTTGGCCAGGTACATGTTT |

TABLE 3
List of shRNAs Used in This Study

| SEQ ID | Gene/ | | |
|---|---|---|---|
| NO. | shRNA | Oligo ID | Sequence (5′ → 3′) |
| 57 | FLI1 | TRCN0000005322 | CGTCATGTTCTGGTTTGAGAT |
| 58 | EWSR1 | V3LHS_641854 | ACAGCCAAGCTCCAAGTCA |
| 59 | EWSRI | V3LHS_376291 | AGCAGAGTAGCTATGGTCA |
| 60 | CIGALT1 | TRCN0000035411 | CCCAGCCTAATGTTCTTCATA |
| 61 | CIGALT1 | TRCN0000035409 | GCGTTGTAACAAAGTGTTGTT |
| 62 | C1GALT1C1 | TRCN0000034990 | CCCACTACGTTTGCTATCATT |
| 63 | C1GALT1C1 | TRCN0000034991 | CCTTAGGGCATTTGGGCATAT |
| 64 | CIGALT1(M) | TRCN0000110365 | CGTGTCTTGATACAGTAATAT |
| 65 | SMO(M) | TRCN0000026245 | CTCGGGCAAGACATCCTATTT |
| 66 | SMO | TRCN0000014364 | GTGGAGAAGATCAACCTGTTT |
| 67 | H3C10 | TRCN0000106785 | CCCATCGCTATCGGCCTGGTA |
| 68 | H3C10 | TRCN0000106788 | GCAGGACTTCAAGACCGACTT |
| 69 | ZBTB21 | TRCN0000019987 | CGCTTGTGACATCTGTCACAA |
| 70 | ZBTB21 | TRCN0000019988 | CCTTTCTGACTAACATCGTTT |
| 71 | HIRA | TRCN0000020514 | GCAGTGACGATTGAGCCATTT |
| 72 | HIRA | TRCN0000020515 | CGTGGATAACACTGTCGTCAT |
| 73 | ATF6B | V3LHS_319349 | ACCAGGGTGATTCACATCA |
| 74 | ATF6B | V3LHS_319348 | TCCTCAGATGTCCAGACCA |
| 75 | LAT | TRCN0000029734 | CCTCAGATAGTTTGTATCCAA |
| 76 | LAT | TRCN0000153024 | GCAAAGCCAGAACTATCACAT |
| 77 | DOCK11 | TRCN0000123018 | GCTACATACAACAGGGAATTT |
| 78 | DOCK11 | TRCN0000123016 | CCAGGCTACTTGAATCTGAAT |
| 79 | FUT3 | TRCN0000035864 | GCGCTGGATCTGGTTCAACTT |
| 80 | FUT3 | TRCN0000035865 | CGCACTGCTATTTCAGCTGCT |
| 81 | HMGCL | TRCN0000078273 | GCCAAGGAAGTAGTCATCTTT |
| 82 | HMGCL | TRCN0000078276 | CTGATAGACATGCTTTCTGAA |

(M): Mus musculus (mouse)

### REFERENCES

1. Varki, A., Cummings, R. D., Esko, J. D., Stanley, P., Hart, G. W., Aebi, M., Mohnen, D., Kinoshita, T., Packer, N.H., Prestegard, J. H., Schnaar, R. L., and Seeberger, P. H., editors. (2022). Essentials of Glycobiology, 4th edition (Cold Spring Harbor (NY): Cold Spring Harbor Laboratory Press).
2. Bangs, F., and Anderson, K.V. (2017). Primary Cilia and Mammalian Hedgehog Signaling. Cold Spring Harb Perspect Biol 9.
3. Beauchamp, E., Bulut, G., Abaan, O., Chen, K., Merchant, A., Matsui, W., Endo, Y., Rubin, J.S., Toretsky, J., and Uren, A. (2009). GLI1 is a direct transcriptional target of EWS-FLI1 oncoprotein. J Biol Chem 284, 9074-9082.
4. Benjamini, Y., and Hochberg, Y. (1995). Controlling the false discovery rate: a practical and powerful approach to multiple testing. J Roy Statist Soc Ser B 57, 289-300.
5. Campeau, E., Ruhl, V. E., Rodier, F., Smith, C. L., Rahmberg, B. L., Fuss, J. O., Campisi, J., Yaswen, P., Cooper, P. K., and Kaufman, P. D. (2009). A versatile viral system for expression and depletion of proteins in mammalian cells. PLOS One 4, e6529.
6. Capes-Davis, A., Theodosopoulos, G., Atkin, I., Drexler, H. G., Kohara, A., MacLeod, R. A., Masters, J. R., Nakamura, Y., Reid, Y. A., Reddel, R. R., Freshney, R. I. (2010). Check your cultures! A list of cross-contaminated or misidentified cell lines. Int J Cancer. 127 (1): 1-8.
7. Casey, D. L., Lin, T. Y., and Cheung, N. V. (2019). Exploiting Signaling Pathways and Immune Targets Beyond the Standard of Care for Ewing Sarcoma. Front Oncol 9, 537.
8. Castillero-Trejo, Y., Eliazer, S., Xiang, L., Richardson, J. A., and Ilaria, R. L., Jr. (2005). Expression of the EWS/FLI-1 oncogene in murine primary bone-derived cells Results in EWS/FLI-1-dependent, ewing sarcoma-like tumors. Cancer Res 65, 8698-8705.
9. Chen, J. K., Taipale, J., Young, K. E., Maiti, T., and Beachy, P. A. (2002). Small molecule modulation of Smoothened activity. Proc Natl Acad Sci USA 99, 14071-14076.
10. Chou, C. H., Huang, M. J., Liao, Y. Y., Chen, C. H., and Huang, M. C. (2017). C1GALT1 Seems to Promote In Vitro Disease Progression in Ovarian Cancer. Int J Gynecol Cancer 27, 863-871.
11. Cidre-Aranaz, F., and Alonso, J. (2015). EWS/FLI1 Target Genes and Therapeutic Opportunities in Ewing Sarcoma. Front Oncol 5, 162.
12. Cong, L., Ran, F. A., Cox, D., Lin, S., Barretto, R., Habib, N., Hsu, P. D., Wu, X., Jiang, W., Marraffini, L. A., et al. (2013). Multiplex genome engineering using CRISPR/Cas systems. Science 339, 819-823.
13. Deng, H., Huang, L., Liao, Z., Liu, M., Li, Q., and Xu, R. (2020). Itraconazole inhibits the Hedgehog signaling pathway thereby inducing autophagy-mediated apoptosis of colon cancer cells. Cell Death Dis 11, 539.
14. Desai, P. B., Stuck, M.W., Lv, B., and Pazour, G. J. (2020). Ubiquitin links smoothened to intraflagellar transport to regulate Hedgehog signaling. J Cell Biol 219.
15. Doench, J. G., Hartenian, E., Graham, D. B., Tothova, Z., Hegde, M., Smith, I., Sullender, M., Ebert, B. L., Xavier, R. J., and Root, D. E. (2014). Rational design of highly active sgRNAs for CRISPR-Cas9-mediated gene inactivation. Nat Biotechnol 32, 1262-1267.
16. Erkizan, H. V., Scher, L. J., Gamble, S. E., Barber-Rotenberg, J. S., Sajwan, K. P., Uren, A., and Toretsky, J. A. (2011). Novel peptide binds EWS-FLI1 and reduces the oncogenic potential in Ewing tumors. Cell Cycle 10, 3397-3408.
17. Evers, B., Jastrzebski, K., Heijmans, J. P., Grernrum, W., Beijersbergen, R. L., and Bernards, R. (2016). CRISPR knockout screening outperforms shRNA and CRISPRi in identifying essential genes. Nat Biotechnol 34, 631-633.
18. Flores, G., and Grohar, P. J. (2021). One oncogene, several vulnerabilities: EWS/FLI targeted therapies for Ewing sarcoma. J Bone Oncol 31, 100404.
19. Fu, C., Zhao, H., Wang, Y., Cai, H., Xiao, Y., Zeng, Y., and Chen, H. (2016). Tumor-associated antigens: Tn antigen, sTn antigen, and T antigen. HLA 88, 275-286.
20. Gierisch, M. E., Pedot, G., Walser, F., Lopez-Garcia, L. A., Jaaks, P., Niggli, F. K., and Schafer, B.W. (2019). USP19 deubiquitinates EWS-FLI1 to regulate Ewing sarcoma growth. Sci Rep 9, 951.
21. Grohar, P. J., Kim, S., Rangel Rivera, G. O., Sen, N., Haddock, S., Harlow, M. L., Maloney, N. K., Zhu, J., O'Neill, M., Jones, T. L., et al. (2016). Functional Genomic Screening Reveals Splicing of the EWS-FLI1 Fusion Transcript as a Vulnerability in Ewing Sarcoma. Cell Rep 14, 598-610.
22. Gu, D., Schlotman, K. E., and Xie, J. (2016). Deciphering the role of hedgehog signaling in pancreatic cancer. J Biomed Res 30, 353-360.
23. Gurdziel, K., Vogt, K. R., Schneider, G., Richards, N., and Gumucio, D. L. (2016). Computational prediction and experimental validation of novel Hedgehog-responsive enhancers linked to genes of the Hedgehog pathway. BMC Dev Biol 16, 4.
24. Hesla, A. C., Papakonstantinou, A., and Tsagkozis, P. (2021). Current Status of Management and Outcome for Patients with Ewing Sarcoma. Cancers (Basel) 13.
25. Hu-Lieskovan, S., Heidel, J. D., Bartlett, D. W., Davis, M.E., and Triche, T. J. (2005). Sequence-specific knockdown of EWS-FLI1 by targeted, nonviral delivery of small interfering RNA inhibits tumor growth in a murine model of metastatic Ewing's sarcoma. Cancer Res 65, 8984-8992.
26. Isoherranen, N., Kunze, K. L., Allen, K. E., Nelson, W. L., and Thummel, K. E. (2004). Role of itraconazole metabolites in CYP3A4 inhibition. Drug Metab Dispos 32, 1121-1131.
27. Joo, J., Christensen, L., Warner, K., States, L., Kang, H. G., Vo, K., Lawlor, E. R., and May, W. A. (2009). GLI1 is a central mediator of EWS/FLI1 signaling in Ewing tumors. PLOS One 4, e7608.
28. Ju, T., and Cummings, R.D. (2002). A unique molecular chaperone Cosmc required for activity of the mammalian core 1 beta 3-galactosyltransferase. Proc Natl Acad Sci USA 99, 16613-16618.
29. Kamitani, T., Kito, K., Nguyen, H.P., Yeh, E. T. (1997). Characterization of NEDD8, a developmentally down-regulated ubiquitin-like protein. J Biol Chem. 272, 28557-28562.
30. Kassambara, A., Kosinski, M., Biecek, P., and Fabian, S. (2021). Survminer.
31. Kim, J., Aftab, B. T., Tang, J. Y., Kim, D., Lee, A. H., Rezaee, M., Kim, J., Chen, B., King, E. M., Borodovsky, A., et al. (2013). Itraconazole and arsenic trioxide inhibit Hedgehog pathway activation and tumor growth associated with acquired resistance to smoothened antagonists. Cancer Cell 23, 23-34.
32. Kim, J., Lee, J. J., Kim, J., Gardner, D., and Beachy, P. A. (2010a). Arsenic antagonizes the Hedgehog pathway by preventing ciliary accumulation and reducing stability of the Gli2 transcriptional effector. Proc Natl Acad Sci USA 107, 13432-13437.
33. Kim, J., Tang, J. Y., Gong, R., Kim, J., Lee, J. J., Clemons, K. V., Chong, C. R., Chang, K. S., Fereshteh, M., Gardner, D., et al. (2010b). Itraconazole, a commonly used antifungal that inhibits Hedgehog pathway activity and cancer growth. Cancer Cell 17, 388-399.
34. Kinzler, K. W., and Vogelstein, B. (1990). The GLI gene encodes a nuclear protein which binds specific sequences in the human genome. Mol Cell Biol 10, 634-642.
35. Kuo, T. C., Wu, M. H., Yang, S. H., Chen, S. T., Hsu, T. W., Jhuang, J. Y., Liao, Y. Y., Tien, Y. W., and Huang, M. C. (2021). C1GALT1 high expression is associated with poor survival of patients with pancreatic ductal adenocarcinoma and promotes cell invasiveness through integrin alphav. Oncogene 40, 1242-1254.
36. Langmead, B., Trapnell, C., Pop, M., and Salzberg, S. L. (2009). Ultrafast and memory-efficient alignment of short DNA sequences to the human genome. Genome Biol 10, R25.
37. Lauth, M., Bergstrom, A., Shimokawa, T., and Toftgard, R. (2007). Inhibition of GLI-mediated transcription and tumor cell growth by small-molecule antagonists. Proc Natl Acad Sci USA 104, 8455-8460.
38. Lezot, F., Corre, I., Morice, S., Redini, F., and Verrecchia, F. (2020). SHH Signaling Pathway Drives Pediatric Bone Sarcoma Progression. Cells 9.
39. Li, K., Fang, D., Xiong, Z., and Luo, R. (2019). Inhibition of the hedgehog pathway for the treatment of cancer using Itraconazole. Onco Targets Ther 12, 6875-6886.
40. Li, Y., Pasunooti, K. K., Li, R.J., Liu, W., Head, S. A., Shi, W. Q., and Liu, J. O. (2018). Novel Tetrazole-Containing Analogues of Itraconazole as Potent Antiangiogenic Agents with Reduced Cytochrome P450 3A4 Inhibition. J Med Chem 61, 11158-11168.
41. Li, Y., Pasunooti, K. K., Peng, H., Li, R. J., Shi, W. Q., Liu, W., Cheng, Z., Head, S. A., and Liu, J. O. (2020). Design and Synthesis of Tetrazole- and Pyridine-Containing Itraconazole Analogs as Potent Angiogenesis Inhibitors. ACS Med Chem Lett 11, 1111-1117.
42. Liang, G., Liu, M., Wang, Q., Shen, Y., Mei, H., Li, D., and Liu, W. (2017). Itraconazole exerts its anti-melanoma effect by suppressing Hedgehog, Wnt, and PI3K/mTOR signaling pathways. Oncotarget 8, 28510-28525.
43. Liao, X., Siu, M. K., Au, C. W., Wong, E. S., Chan, H. Y., Ip, P. P., Ngan, H. Y., and Cheung, A. N. (2009). Aberrant activation of hedgehog signaling pathway in ovarian cancers: effect on prognosis, cell invasion and differentiation. Carcinogenesis 30, 131-140.
44. Lin, M. C., Chien, P. H., Wu, H. Y., Chen, S. T., Juan, H. F., Lou, P. J., and Huang, M. C. (2018). C1GALT1 predicts poor prognosis and is a potential therapeutic target in head and neck cancer. Oncogene 37, 5780-5793.
45. Liu, H., Gu, D., and Xie, J. (2011). Clinical implications of hedgehog signaling pathway inhibitors. Chin J Cancer 30, 13-26.
46. Loganathan, S. N., Tang, N., Fleming, J. T., Ma, Y., Guo, Y., Borinstein, S. C., Chiang, C., and Wang, J. (2016). BET bromodomain inhibitors suppress EWS-FLI1-dependent transcription and the IGF1 autocrine mechanism in Ewing sarcoma. Oncotarget 7, 43504-43517.
47. Lu, B., Klingbeil, O., Tarumoto, Y., Somerville, T. D. D., Huang, Y. H., Wei, Y., Wai, D. C., Low, J. K. K., Milazzo, J. P., Wu, X. S., et al. (2018). A Transcription Factor Addiction in Leukemia Imposed by the MLL Promoter Sequence. Cancer Cell 34, 970-981 e978.
48. May, W. A., Gishizky, M. L., Lessnick, S. L., Lunsford, L. B., Lewis, B. C., Delattre, O., Zucman, J., Thomas, G., and Denny, C. T. (1993). Ewing sarcoma 11; 22 translocation produces a chimeric transcription factor that requires the DNA-binding domain encoded by FLI1 for transformation. Proc Natl Acad Sci USA 90, 5752-5756.
49. Menche, J., Guney, E., Sharma, A., Branigan, P.J., Loza, M. J., Baribaud, F., Dobrin, R., and Barabasi, A. L. (2017). Integrating personalized gene expression profiles into predictive disease-associated gene pools. NPJ Syst Biol Appl 3, 10.
50. Ng, P. K., Li, J., Jeong, K. J., Shao, S., Chen, H., Tsang, Y. H., Sengupta, S., Wang, Z., Bhavana, V. H., Tran, R., et al. (2018). Systematic Functional Annotation of Somatic Mutations in Cancer. Cancer Cell 33, 450-462 e410.
51. Nguyen, N. M., and Cho, J. (2022). Hedgehog Pathway Inhibitors as Targeted Cancer Therapy and Strategies to Overcome Drug Resistance. Int J Mol Sci 23.
52. Pace, J. R., Teske, K. A., Chau, L .Q., Wechsler-Reya, R. J., and Hadden, M. K. (2019) Structure-activity relationships for itraconazole-based triazolone analogues as Hedgehog pathway Inhibitors. J Med Chem 62, 3873-3885.
53. Pedersen, T. (2022). patchwork: The Composer of Plots.
54. Penet, M. F., Shah, T., Bharti, S., Krishnamachary, B., Artemov, D., Mironchik, Y., Wildes, F., Maitra, A., and Bhujwalla, Z. M. (2015). Metabolic imaging of pancreatic ductal adenocarcinoma detects altered choline metabolism. Clin Cancer Res 21, 386-395.
55. Pereira, M. E., Kabat, E. A., Lotan, R., and Sharon, N. (1976). Immunochemical studies on the specificity of the peanut (Arachis hypogaea) agglutinin. Carbohydr Res 51, 107-118.
56. Peukert, S., Jain, R. K., Geisser, A., Sun, Y., Zhang, R., Bourret, A., Carlson, A., Dasilva, J., Ramamurthy, A., and Kelleher, J. F. (2009). Identification and structure-activity relationships of ortho-biphenyl carboxamides as potent Smoothened antagonists inhibiting the Hedgehog signaling pathway. Bioorg Med Chem Lett 19, 328-331.
57. Pusapati, G. V., Kong, J.H., Patel, B. B., Gouti, M., Sagner, A., Sircar, R., Luchetti, G., Ingham, P. W., Briscoe, J., and Rohatgi, R. (2018a). G protein-coupled receptors control the sensitivity of cells to the morphogen Sonic Hedgehog. Sci Signal 11.
58. Pusapati, G. V., Kong, J.H., Patel, B. B., Krishnan, A., Sagner, A., Kinnebrew, M., Briscoe, J., Aravind, L., and Rohatgi, R. (2018b). CRISPR Screens Uncover Genes that Regulate Target Cell Sensitivity to the Morphogen Sonic Hedgehog. Dev Cell 44, 271.
59. Riggi, N., Cironi, L., Provero, P., Suva, M. L., Kaloulis, K., Garcia-Echeverria, C., Hoffmann, F., Trumpp, A., and Stamenkovic, I. (2005). Development of Ewing's sarcoma from primary bone marrow-derived mesenchymal progenitor cells. Cancer Res 65, 11459-11468.
60. Riggi, N., Suva, M. L., and Stamenkovic, I. (2021). Ewing's Sarcoma. N Engl J Med 384, 154-164.
61. Rimkus, T .K., Carpenter, R. L., Qasem, S., Chan, M., and Lo, H. W. (2016). Targeting the Sonic Hedgehog Signaling Pathway: Review of Smoothened and GLI Inhibitors. Cancers (Basel) 8.
62. Sanjana, N. E., Shalem, O., and Zhang, F. (2014). Improved vectors and genome-wide libraries for CRISPR screening. Nat Methods 11, 783-784.
63. Sasaki, H., Hui, C., Nakafuku, M., and Kondoh, H. (1997). A binding site for Gli proteins is essential for HNF-3beta floor plate enhancer activity in transgenics and can respond to Shh in vitro. Development 124, 1313-1322.
64. Savola, S., Klami, A., Myllykangas, S., Manara, C., Scotlandi, K., Picci, P., Knuutila, S., and Vakkila, J. (2011). High Expression of Complement Component 5 (C₅) at Tumor Site Associates with Superior Survival in Ewing's Sarcoma Family of Tumour Patients. ISRN Oncol 2011, 168712.
65. Shi, W., Nacev, B. A., Aftab, B. T., Head, S., Rudin, C. M., and Liu, J. O. (2011). Itraconazole side chain analogues: structure-activity relationship studies for inhibition of endothelial cell proliferation, vascular endothelial growth factor receptor 2 (VEGFR2) glycosylation, and hedgehog signaling. J Med Chem 54, 7363-7374.
66. Shukla, N., Somwar, R., Smith, R.S., Ambati, S., Munoz, S., Merchant, M., D'Arcy, P., Wang, X., Kobos, R., Antczak, C., et al. (2016). Proteasome Addiction Defined in Ewing Sarcoma Is Effectively Targeted by a Novel Class of 19S Proteasome Inhibitors. Cancer Res 76, 4525-4534.
67. Skoda, A. M., Simovic, D., Karin, V., Kardum, V., Vranic, S., and Serman, L. (2018). The role of the Hedgehog signaling pathway in cancer: A comprehensive review. Bosn J Basic Med Sci 18, 8-20.
68. Smith, R., Owen, L. A., Trem, D. J., Wong, J. S., Whangbo, J. S., Golub, T. R., and Lessnick, S. L. (2006). Expression profiling of EWS/FLI identifies NKX2.2 as a critical target gene in Ewing's sarcoma. Cancer Cell 9, 405-416.
69. Soriano, P., Friedrich, G., and Lawinger, P. (1991). Promoter interactions in retrovirus vectors introduced into fibroblasts and embryonic stem cells. J Virol 65, 2314-2319.
70. Sun, X., Zhan, M., Sun, X., Liu, W., and Meng, X. (2021). C1GALT1 in health and disease. Oncol Lett 22, 589.
71. Taipale, J., Chen, J. K., Cooper, M. K., Wang, B., Mann, R. K., Milenkovic, L., Scott, M. P., and Beachy, P. A. (2000). Effects of oncogenic mutations in Smoothened and Patched can be reversed by cyclopamine. Nature 406, 1005-1009.
72. Tanaka, K., Iwakuma, T., Harimaya, K., Sato, H., and Iwamoto, Y. (1997). EWS-Fli1 antisense oligodeoxynucleotide inhibits proliferation of human Ewing's sarcoma and primitive neuroectodermal tumor cells. J Clin Invest 99, 239-247.
73. Therneau, T. (2023). A package for survival analysis in R. R package version 3.5-5. (https://CRAN.R-project.org/package=survival).
74. Therneau, T. M., and Grambsch, P. M. (2000). Modeling Survival Data: Extending the Cox Model. (Springer-Verlag, New York).
75. Tsubamoto, H., Inoue, K., Sakata, K., Ueda, T., Takeyama, R., Shibahara, H., and Sonoda, T. (2017). Itraconazole Inhibits AKT/mTOR Signaling and Proliferation in Endometrial Cancer Cells. Anticancer Res 37, 515-519.
76. Uren, A., and Toretsky, J. A. (2005). Ewing's sarcoma oncoprotein EWS-FLI1: the perfect target without a therapeutic agent. Future Oncol 1, 521-528.
77. Waldner, C., Roose, M., and Ryffel, G. U. (2009). Red fluorescent Xenopus laevis: a new tool for grafting analysis. BMC Dev Biol 9, 37.
78. Wan, Y., and Yu, L.G. (2021). Expression and Impact of C₁GalT1 in Cancer Development and Progression. Cancers (Basel) 13.
79. Wajapeyee, N., Malonia, S. K., Palakurthy, R. K., Green, M. R. (2013). Oncogenic RAS directs silencing of tumor suppressor genes through ordered recruitment of transcriptional repressors. Genes Dev. 27, 2221-2226.
80. Wen, J., Teske, K. A., and Kyle Hadden, M. (2020). Inhibition of hedgehog signaling by stereochemically defined des-triazole itraconazole analogues. Bioorg Med Chem Lett 30, 126794.
81. Winklmayr, M., Schmid, C., Laner-Plamberger, S., Kaser, A., Aberger, F., Eichberger, T., and Frischauf, A. M. (2010). Non-consensus GLI binding sites in Hedgehog target gene regulation. BMC Mol Biol 11, 2.
82. Xia, T., Xiang, T., and Xie, H. (2022). Update on the role of C1GALT1 in cancer. Oncol Lett 23, 97.
83. Yang, W., Ao, M., Hu, Y., Li, Q. K., and Zhang, H. (2018). Mapping the O-glycoproteome using site-specific extraction of O-linked glycopeptides (EXOO). Mol Syst Biol 14, e8486.
84. Yang, W., Ao, M., Song, A., Xu, Y., Sokoll, L., and Zhang, H. (2020). Mass Spectrometric Mapping of Glycoproteins Modified by Tn-Antigen Using Solid-Phase Capture and Enzymatic Release. Anal Chem 92, 9230-9238.
85. Zhang, J., Liu, Z., and Jia, J. (2021). Mechanisms of Smoothened Regulation in Hedgehog Signaling. Cells 10.
86. Zwerner, J. P., Joo, J., Warner, K. L., Christensen, L., Hu-Lieskovan, S., Triche, T. J., and May, W. A. (2008). The EWS/FLI1 oncogenic transcription factor deregulates GLI1. Oncogene 27, 3282-3291.
87. Im, Y. H., Kim, H.T., Lee, C., Poulin, D., Welford, S., Sorensen, P.H., Denny, C.T., Kim, S.J. (2000). EWS-FLI1, EWS-ERG, and EWS-ETVI oncoproteins of Ewing tumor family all suppress transcription of transforming growth factor beta type II receptor gene. Cancer Res. 60, 1536-40.
88. Greulich, B. M., Rajendran, S., Downing, N. F., Nicholas, T .R., Hollenhorst, P. C. (2023). A complex with poly (A)-binding protein and EWS facilitates the transcriptional function of oncogenic ETS transcription factors in prostate cells. J Biol Chem. 299, 105453.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A method of treating Ewing sarcoma in a subject, comprising administering to the subject an effective amount of an agent or a pharmaceutically acceptable salt thereof that:
a) reduces expression of core 1 synthase, glycoprotein-N-acetylgalactosamine 3-beta-galactosyltransferase 1 (C1GALT1);
b) reduces activity of C1GALT1;
or both a) and b), thereby treating Ewing sarcoma in the subject.

2. The method of claim 1, wherein the agent or the pharmaceutically acceptable salt thereof reduces expression of C1GALT1.

3. The method of claim 1, wherein the agent or the pharmaceutically acceptable salt thereof reduces activity of C1GALT1.

4. The method of claim 1, wherein the agent or the pharmaceutically acceptable salt thereof:
a) reduces expression of Smoothened (SMO);
b) reduces O—N-acetylgalactosamine-glycosylation (O-GalNAc-glycosylation) of SMO;
c) reduces activity of SMO;
d) reduces stability of SMO;
e) increases ubiquitination of SMO;
f) reduces ligand-independent hedgehog signaling;
g) reduces expression of GLI family zinc finger 1 (GLI1);
h) reduces expression of GLI family zinc finger 2 (GLI2);
i) reduces binding of GLI1 to EWS RNA binding protein 1 (EWSR1);
j) reduces binding of GLI2 to EWSR1;
k) reduces binding of GLI1 to EWSR1 and Fli-1 proto-oncogene, ETS transcription factor (FLI1) fusion gene (EWSR1::FLI1);
l) reduces binding of GLI2 to EWSR1::FLI1;
m) reduces expression of EWSR1::FLI1;
n) reduces expression of an EWSR1-FLI1 fusion protein (EWSR1::FLI1) target gene;
o) reduces binding of GLI1 to EWSR1 and ETS transcription factor ERG (ERG) fusion gene (EWSR1::ERG);
p) reduces binding of GLI2 to EWSR1::ERG;
q) reduces expression of EWSR1::ERG;
r) reduces expression of an EWSR1-ERG fusion protein (EWSR1::ERG) target gene;
or any combination of the foregoing.

5. The method of claim 4, wherein the agent or the pharmaceutically acceptable salt thereof reduces expression of EWSR1::FLI1, EWSR1::ERG, or both, and:
a) reduces expression of H3 clustered histone 10 (H3C10), zinc finger and BTB domain containing 21 (ZBTB21), histone cell cycle regulator (HIRA), activating transcription factor 6 beta (ATF6B), linker for activation of T cells (LAT), dedicator of cytokinesis 11 (DOCK11), fucosyltransferase 3 (FUT3), 3-hydroxy-3-methylglutaryl-CoA lyase (HMGCL), C1GALT1, C1GALT1-specific chaperone 1 (C1GALT1C1), SMO, GLI1, GLI2 or any combination of the foregoing;
b) reduces activity of H3C10, ZBTB21, HIRA, ATF6B, LAT, DOCK11, FUT3, HMGCL, C1GALT1, C1GALT1C1, SMO, GLI1, GLI2 or any combination of the foregoing;
or both a) and b).

6. The method of claim 4, wherein the EWSR1::FLI1 target gene comprises G protein-coupled receptor kinase 5 (GRK5), nuclear receptor subfamily 0 group b member 1 (NR0B1), vaccinia-related kinase 1 (VRK1), cyclin-dependent kinase inhibitor 1A (CDKN1A), fos-related antigen 2 (FOSL2), forkhead box protein 01 (FOXO1), or any combination of the foregoing.

7. The method of claim 1, wherein the agent comprises itraconazole or an analog thereof.

8. The method of claim 1, wherein the agent or the pharmaceutically acceptable salt thereof is administered to the subject at about 0.0001 to about 100 mg per kilogram of body weight per day.

9. The method of claim 1, wherein the agent or the pharmaceutically acceptable salt thereof is administered to the subject:
a) at about 100 to about 1,200 mg per day;
b) at about 100 mg, about 200 mg, about 300 mg, about 400 mg, about 500 mg, or about 600 mg, once daily; or
c) at about 100 mg, about 200 mg, about 300 mg, about 400 mg, about 500 mg, or about 600 mg, twice daily.

10. The method of claim 1, wherein the agent or the pharmaceutically acceptable salt thereof is administered to the subject at about 10 mg/mL.

11. The method of claim 1, wherein the agent or the pharmaceutically acceptable salt thereof is administered to the subject:
a) for at least 1, 2, 3, 4, 5, 6, 7, 8, 9, 10, 11 or 12 weeks;
b) for about 1 day to about 12 weeks;
or both a) and b).

12. The method of claim 1, further comprising administering to the subject a second agent, a chemotherapy, a radiation therapy, or any combination of the foregoing.

13. The method of claim 1, wherein the amount of the agent or the pharmaceutically acceptable salt thereof administered to the subject is sufficient to reduce tumor size, improve radiological response, prolong event-free survival, prolong disease-free survival, prolong metastasis-free survival, prolong overall survival, or any combination of the foregoing.

14. A method of treating Ewing sarcoma in a subject, comprising administering to the subject an effective amount of itraconazole, an analog thereof, or a pharmaceutically acceptable salt thereof.

15. The method of claim 1, wherein the subject is a mammal, optionally wherein the mammal is a human.

16. The method of claim 1, wherein the subject is newly diagnosed with metastatic Ewing sarcoma, non-metastatic Ewing sarcoma, or localized Ewing sarcoma.

17. The method of claim 1, wherein the subject has or is susceptible to metastatic Ewing sarcoma, relapsed or recurrent Ewing sarcoma, non-metastatic Ewing sarcoma, or localized Ewing sarcoma.

18. A method of killing or reducing proliferation of an Ewing sarcoma cell, comprising contacting the cell with an agent or a pharmaceutically acceptable salt thereof that:
a) reduces expression of C1GALT1;
b) reduces activity of C1GALT1;
or both a) and b).

19. The method of claim 18, wherein the agent comprises itraconazole or an analog thereof.

20. The method of claim 18, wherein the cell is a relapsed or recurrent Ewing sarcoma cell, a metastatic Ewing sarcoma cell, a non-metastatic Ewing sarcoma cell, a localized Ewing sarcoma cell, an Ewing sarcoma cell from a subject, an Ewing sarcoma cell from a cell line, or a combination of the foregoing.


SEQUENCE LISTING (90 sequences)

SEQ ID NO: 1 - DNA, 26 bp, Synthetic construct
  source 1..26: mol_type = other DNA
cgacggcgcgccgcaattcctcctgg

SEQ ID NO: 2 - DNA, 26 bp, Synthetic construct
  source 1..26: mol_type = other DNA
catcgctagcgtacgtgggtgttagg

SEQ ID NO: 3 - DNA, 26 bp, Synthetic construct
  source 1..26: mol_type = other DNA
cgacctcgagattttcacccaactgg

SEQ ID NO: 4 - DNA, 26 bp, Synthetic construct
  source 1..26: mol_type = other DNA
catcgcggccgcaagcgattgattcg

SEQ ID NO: 5 - DNA, 22 bp, Homo sapiens
  source 1..22: mol_type = other DNA
gtgacaaatagaggtgggaagc

SEQ ID NO: 6 - DNA, 20 bp, Synthetic construct
  source 1..20: mol_type = other DNA
caccttgaagcgcatgaact

SEQ ID NO: 7 - DNA, 22 bp, Homo sapiens
  source 1..22: mol_type = other DNA
gaagcttatggcagagatgcac

SEQ ID NO: 8 - DNA, 22 bp, Homo sapiens
  source 1..22: mol_type = other DNA
cagtgctttccaaatagatggg

SEQ ID NO: 9 - DNA, 20 bp, Homo sapiens
  source 1..20: mol_type = other DNA
tcatgcaaggcattcagatg

SEQ ID NO: 10 - DNA, 20 bp, Homo sapiens
  source 1..20: mol_type = other DNA
atgggttcttcagggtcgta

SEQ ID NO: 11 - DNA, 22 bp, Homo sapiens
  source 1..22: mol_type = other DNA
atgagcatcatcacctacaagc

SEQ ID NO: 12 - DNA, 22 bp, Homo sapiens
  source 1..22: mol_type = other DNA
cagcccaaagactcacatcttt

SEQ ID NO: 13 - DNA, 22 bp, Homo sapiens
  source 1..22: mol_type = other DNA
aggtggacctggagactctcag

SEQ ID NO: 14 - DNA, 22 bp, Homo sapiens
  source 1..22: mol_type = other DNA
tcctcttggagaagatcagccg

SEQ ID NO: 15 - DNA, 20 bp, Homo sapiens
  source 1..20: mol_type = other DNA
ctgcccaaatgaaggaacat

SEQ ID NO: 16 - DNA, 20 bp, Homo sapiens
  source 1..20: mol_type = other DNA
cacattgcagcctcagaaaa

SEQ ID NO: 17 - DNA, 18 bp, Homo sapiens
  source 1..18: mol_type = other DNA
atgggtcgcatgggaggc

SEQ ID NO: 18 - DNA, 23 bp, Homo sapiens
  source 1..23: mol_type = other DNA
gccttacactggttgcactctgt

SEQ ID NO: 19 - DNA, 23 bp, Homo sapiens
  source 1..23: mol_type = other DNA
agttacccaccccaaactggatc

SEQ ID NO: 20 - DNA, 20 bp, Homo sapiens
  source 1..20: mol_type = other DNA
gcagctccaggaggaattgc

SEQ ID NO: 21 - DNA, 18 bp, Homo sapiens
  source 1..18: mol_type = other DNA
cggacaatcgtctgagcg

SEQ ID NO: 22 - DNA, 16 bp, Homo sapiens
  source 1..16: mol_type = other DNA
ccgtatgatggggggc

SEQ ID NO: 23 - DNA, 21 bp, Homo sapiens
  source 1..21: mol_type = other DNA
tggtcaagagcgtgccctact

SEQ ID NO: 24 - DNA, 20 bp, Homo sapiens
  source 1..20: mol_type = other DNA
cccgctcttgccaccctctg

SEQ ID NO: 25 - DNA, 22 bp, Homo sapiens
  source 1..22: mol_type = other DNA
gctggaaactgcagcaggaatc

SEQ ID NO: 26 - DNA, 19 bp, Homo sapiens
  source 1..19: mol_type = other DNA
tcaggtgaaccaagccgct

SEQ ID NO: 27 - DNA, 20 bp, Homo sapiens
  source 1..20: mol_type = other DNA
tgcaccaccaactgcttagc

SEQ ID NO: 28 - DNA, 21 bp, Homo sapiens
  source 1..21: mol_type = other DNA
ggcatggactgtggtcatgag

SEQ ID NO: 29 - DNA, 27 bp, Homo sapiens
  source 1..27: mol_type = other DNA
cagctacatcaactccggccaataggg

SEQ ID NO: 30 - DNA, 24 bp, Homo sapiens
  source 1..24: mol_type = other DNA
tgctgcggcgttcaagagagactg

SEQ ID NO: 31 - DNA, 18 bp, Homo sapiens
  source 1..18: mol_type = other DNA
gaccacacagacgacttc

SEQ ID NO: 32 - DNA, 22 bp, Homo sapiens
  source 1..22: mol_type = other DNA
cgttcagctccttaaagcattc

SEQ ID NO: 33 - DNA, 22 bp, Homo sapiens
  source 1..22: mol_type = other DNA
ttcagcgtctggtgcgtgagat

SEQ ID NO: 34 - DNA, 22 bp, Homo sapiens
  source 1..22: mol_type = other DNA
caggttggtgtcctcaaagagc

SEQ ID NO: 35 - DNA, 20 bp, Homo sapiens
  source 1..20: mol_type = other DNA
cggtggtcaaacagtgggat

SEQ ID NO: 36 - DNA, 20 bp, Homo sapiens
  source 1..20: mol_type = other DNA
atggagacacaccgccattg

SEQ ID NO: 37 - DNA, 24 bp, Homo sapiens
  source 1..24: mol_type = other DNA
tctgggcccaagatggcagcaatg

SEQ ID NO: 38 - DNA, 22 bp, Homo sapiens
  source 1..22: mol_type = other DNA
ctgaggcagctccaagatgact

SEQ ID NO: 39 - DNA, 20 bp, Homo sapiens
  source 1..20: mol_type = other DNA
cagacctgctccccatccca

SEQ ID NO: 40 - DNA, 25 bp, Homo sapiens
  source 1..25: mol_type = other DNA
tcaggaagcaccaccaggtagcctg

SEQ ID NO: 41 - DNA, 20 bp, Homo sapiens
  source 1..20: mol_type = other DNA
cttctgtaccgctgctgctt

SEQ ID NO: 42 - DNA, 21 bp, Homo sapiens
  source 1..21: mol_type = other DNA
gcagaccacctgtggactctt

SEQ ID NO: 43 - DNA, 17 bp, Homo sapiens
  source 1..17: mol_type = other DNA
ggctgctgagtgagaag

SEQ ID NO: 44 - DNA, 22 bp, Homo sapiens
  source 1..22: mol_type = other DNA
ctggttgaagaagtcgtagaag

SEQ ID NO: 45 - DNA, 18 bp, Homo sapiens
  source 1..18: mol_type = other DNA
ccaacgagctgcaaaacc

SEQ ID NO: 46 - DNA, 21 bp, Homo sapiens
  source 1..21: mol_type = other DNA
tgtcatgtagaccagaccccc

SEQ ID NO: 47 - DNA, 18 bp, Homo sapiens
  source 1..18: mol_type = other DNA
tgccctgaatgaggagcg

SEQ ID NO: 48 - DNA, 25 bp, Homo sapiens
  source 1..25: mol_type = other DNA
taaactctgaaagtattcgctgctg

SEQ ID NO: 49 - DNA, 21 bp, Homo sapiens
  source 1..21: mol_type = other DNA
gtgtgcacggctccttggtat

SEQ ID NO: 50 - DNA, 22 bp, Homo sapiens
  source 1..22: mol_type = other DNA
cctggcctgtatcctttgcctg

SEQ ID NO: 51 - DNA, 20 bp, Homo sapiens
  source 1..20: mol_type = other DNA
ctccacctcgaccgttaccg

SEQ ID NO: 52 - DNA, 20 bp, Homo sapiens
  source 1..20: mol_type = other DNA
gcgcgctctgcagtgagtac

SEQ ID NO: 53 - DNA, 21 bp, Homo sapiens
  source 1..21: mol_type = other DNA
ctgaatccattccgcgcacac

SEQ ID NO: 54 - DNA, 22 bp, Homo sapiens
  source 1..22: mol_type = other DNA
ggacccggactagaacaactgc

SEQ ID NO: 55 - DNA, 21 bp, Homo sapiens
  source 1..21: mol_type = other DNA
gcagctacagtgcttacaccg

SEQ ID NO: 56 - DNA, 19 bp, Homo sapiens
  source 1..19: mol_type = other DNA
tccttcgcactcccactgt

SEQ ID NO: 57 - DNA, 21 bp, Homo sapiens
  source 1..21: mol_type = genomic DNA
cgtcatgttctggtttgagat

SEQ ID NO: 58 - DNA, 19 bp, Homo sapiens
  source 1..19: mol_type = genomic DNA
acagccaagctccaagtca

SEQ ID NO: 59 - DNA, 19 bp, Homo sapiens
  source 1..19: mol_type = genomic DNA
agcagagtagctatggtca

SEQ ID NO: 60 - DNA, 21 bp, Homo sapiens
  source 1..21: mol_type = genomic DNA
cccagcctaatgttcttcata

SEQ ID NO: 61 - DNA, 21 bp, Homo sapiens
  source 1..21: mol_type = genomic DNA
gcgttgtaacaaagtgttgtt

SEQ ID NO: 62 - DNA, 21 bp, Homo sapiens
  source 1..21: mol_type = genomic DNA
cccactacgtttgctatcatt

SEQ ID NO: 63 - DNA, 21 bp, Homo sapiens
  source 1..21: mol_type = genomic DNA
ccttagggcatttgggcatat

SEQ ID NO: 64 - DNA, 21 bp, Homo sapiens
  source 1..21: mol_type = genomic DNA
cgtgtcttgatacagtaatat

SEQ ID NO: 65 - DNA, 21 bp, Homo sapiens
  source 1..21: mol_type = genomic DNA
ctcgggcaagacatcctattt

SEQ ID NO: 66 - DNA, 21 bp, Homo sapiens
  source 1..21: mol_type = genomic DNA
gtggagaagatcaacctgttt

SEQ ID NO: 67 - DNA, 21 bp, Homo sapiens
  source 1..21: mol_type = genomic DNA
cccatcgctatcggcctggta

SEQ ID NO: 68 - DNA, 21 bp, Homo sapiens
  source 1..21: mol_type = genomic DNA
gcaggacttcaagaccgactt

SEQ ID NO: 69 - DNA, 21 bp, Homo sapiens
  source 1..21: mol_type = genomic DNA
cgcttgtgacatctgtcacaa

SEQ ID NO: 70 - DNA, 21 bp, Homo sapiens
  source 1..21: mol_type = genomic DNA
cctttctgactaacatcgttt

SEQ ID NO: 71 - DNA, 21 bp, Homo sapiens
  source 1..21: mol_type = genomic DNA
gcagtgacgattgagccattt

SEQ ID NO: 72 - DNA, 21 bp, Homo sapiens
  source 1..21: mol_type = genomic DNA
cgtggataacactgtcgtcat

SEQ ID NO: 73 - DNA, 19 bp, Homo sapiens
  source 1..19: mol_type = genomic DNA
accagggtgattcacatca

SEQ ID NO: 74 - DNA, 19 bp, Homo sapiens
  source 1..19: mol_type = genomic DNA
tcctcagatgtccagacca

SEQ ID NO: 75 - DNA, 21 bp, Homo sapiens
  source 1..21: mol_type = genomic DNA
cctcagatagtttgtatccaa

SEQ ID NO: 76 - DNA, 21 bp, Homo sapiens
  source 1..21: mol_type = genomic DNA
gcaaagccagaactatcacat

SEQ ID NO: 77 - DNA, 21 bp, Homo sapiens
  source 1..21: mol_type = genomic DNA
gctacatacaacagggaattt

SEQ ID NO: 78 - DNA, 21 bp, Homo sapiens
  source 1..21: mol_type = genomic DNA
ccaggctacttgaatctgaat

SEQ ID NO: 79 - DNA, 21 bp, Homo sapiens
  source 1..21: mol_type = genomic DNA
gcgctggatctggttcaactt

SEQ ID NO: 80 - DNA, 21 bp, Homo sapiens
  source 1..21: mol_type = genomic DNA
cgcactgctatttcagctgct

SEQ ID NO: 81 - DNA, 21 bp, Homo sapiens
  source 1..21: mol_type = genomic DNA
gccaaggaagtagtcatcttt

SEQ ID NO: 82 - DNA, 21 bp, Homo sapiens
  source 1..21: mol_type = genomic DNA
ctgatagacatgctttctgaa

SEQ ID NO: 83 - DNA, 20 bp, Homo sapiens
  source 1..20: mol_type = genomic DNA
gcaacactttgttacaacgc

SEQ ID NO: 84 - DNA, 20 bp, Homo sapiens
  source 1..20: mol_type = genomic DNA
gaggtattctaactcataca

SEQ ID NO: 85 - DNA, 20 bp, Homo sapiens
  source 1..20: mol_type = genomic DNA
cgagaccctatccccggtaa

SEQ ID NO: 86 - DNA, 20 bp, Homo sapiens
  source 1..20: mol_type = genomic DNA
aacaactgctgactaatccg

SEQ ID NO: 87 - DNA, 24 bp, Homo sapiens
  source 1..24: mol_type = other DNA
tccaagtcaatatagccaacagag

SEQ ID NO: 88 - DNA, 21 bp, Homo sapiens
  source 1..21: mol_type = other DNA
ctgtggaaggagatggttgag

SEQ ID NO: 89 - DNA, 20 bp, Homo sapiens
  source 1..20: mol_type = genomic DNA
catccctggactgattgtca

SEQ ID NO: 90 - DNA, 20 bp, Homo sapiens
  source 1..20: mol_type = genomic DNA
ggttggccaggtacatgttt